(19) United States
(12) Patent Application Publication
(10) Pub. No.: US 2023/0295657 A1
(43) Pub. Date: Sep. 21, 2023

(54) GENE THERAPY USING NUCLEIC ACID CONSTRUCTS COMPRISING METHYL CPG BINDING PROTEIN 2 (MECP2) PROMOTER SEQUENCES

(71) Applicant: UCB BIOPHARMA SRL, BRUSSELS (BE)

(72) Inventors: NIKITA DALAL, DURHAM, NC (US); AMI KABADI, DURHAM, NC (US); TOSHAL ROHIT PATEL, BRUSSELS (BE); PATRICK MARK DOWNEY, BRUSSELS (BE); AMULYA NIDHI SHRIVASTAVA, BRUSSELS (BE)

(21) Appl. No.: 18/021,005

(22) PCT Filed: Aug. 11, 2021
(86) PCT No.: PCT/EP2021/072365

§ 371 Date: Feb. 13, 2023

Related U.S. Application Data
(60) Provisional application No. 63/064,431, filed on Aug. 12, 2020.

(51) Int. Cl. C12N 15/86; C07K 14/47; A61K 48/00

(52) U.S. Cl. CPC C12N 15/86; C07K 14/4702; A61K 48/0066; C12N 2750/14143; C12N 2830/008

(57) ABSTRACT

The present invention relates to nucleic acid constructs comprising methyl CpG binding protein 2 (MeCP2) promoter sequences. The present invention further relates to vectors, viral vector host cells and pharmaceutical compositions comprising said nucleic acid constructs. The present invention also concerns the therapeutic use of said nucleic acid constructs, vectors, viral vectors and pharmaceutical compositions.

### FIELD OF THE INVENTION

The present invention relates to nucleic acid constructs comprising methyl CpG binding protein 2 (MeCP2) promoter sequences. The present invention further relates to vectors, viral vectors, host cells and pharmaceutical compositions comprising said nucleic acid constructs. The present invention also concerns the therapeutic use of said nucleic acid constructs, vectors, viral vectors and pharmaceutical compositions.

### BACKGROUND OF THE INVENTION

Frontotemporal dementia (FTD) is the second most common type of dementia after Alzheimer's Disease (Olney et al. Neurol. Clin. 2017 May; 35(2): 339-374). A mutation in one allele of the GRN gene, which encodes the protein progranulin (PGRN), is associated with the development of FTD (Baker et al., Nature. 2006 Aug. 24; 442(7105):916-919). Homozygous mutations in GRN are associated with neuronal ceroid lipofuscinosis 11 (NCL11), which is characterized by cerebellar ataxia, seizures, retinitis pigmentosa, and cognitive disorders, usually beginning between 13 and 25 years of age (Faber et al. Brain. 2020; 143(1):303-31).

A variety of mutations can cause a loss of function of PGRN. In PGRN-deficient mouse models, driving neuronal expression of PGRN using an AAV gene therapy approach has been shown to correct behavioral deficits associated with FTD (Arrant et al. Brain. 2017; 140.5: 1447-1465). Thus, there is a strong biological rationale for a therapeutic approach which increases levels of PGRN in tissues and cells of the central nervous system (CNS) to treat neurological diseases associated with PGRN deficiency.

Adeno-associated virus (AAV) vectors are a commonly used vehicle to deliver molecular therapeutics for the treatment of clinical disorders. Many AAV-based therapies are gene replacement therapies. However, in order to provide robust AAV production and transgene expression, the AAV construct comprising the transgene of interest should be between 4.1 kb and 4.7 kb to allow for optimal packaging of the AAV. So-called ‘stuffer sequences’ or inert DNA can be added to the transgene or vector backbone to increase the overall length of the construct. However, vectors are sensitive to stuffer sequences and must therefore be chosen carefully so as not to negatively affect transgene expression, patient immune responses, and AAV packaging efficiency. Another approach to build length into AAV constructs is to modify the transgene sequence itself. However, this approach may not be suitable where it is desirable to use the native (wild type) transgene nucleotide sequence.

A further approach to increase the overall length of AAV constructs is through the inclusion of an engineered promoter sequence. Such promoters must be chosen carefully so as to ensure suitable in vivo transgene expression levels. Moreover, where site-specific transgene expression is required for the treatment of neurological disorders, as in the case of PGRN gene therapy, selection of a promoter which provides targeted expression of the transgene of interest in the desired tissue or cell type is crucial.

Typically, a nucleotide sequence encoding the PGRN coding sequence is about 1.8 kb in length, which is significantly shorter than the optimal length of 4.1 to 4.7 kb for packaging of a nucleic acid construct into an AAV. Thus, there remains a need for promoter sequences which can be used to increase the length of a viral vector construct while at the same time providing robust and CNS-targeted expression of PGRN.

### SUMMARY OF THE INVENTION

It has been found that promoters derived from the methyl-CpG-binding protein 2 (MeCP2) gene are highly effective for driving CNS-targeted expression of PGRN in a gene therapy setting. Such promoters were observed to provide higher PGRN expression and transduction efficiency than equivalent promoters comprising alternative CNS-specific promoters, such as those derived from the neuron-specific enolase 1 (NSE1) gene.

The inventors have also generated engineered MeCP2 promoters of over 2000 bp in length. In addition to a minimal MeCP2 promoter sequence, these engineered MeCP2 promoters comprise an additional intron. The nucleotide sequences of these introns were derived from a naturally occurring stretch of an MECP2 gene (a natural intron) or were constructed by combining disparate sequences derived from an MECP2 gene (a synthetic intron). Gene therapy constructs comprising the engineered MeCP2 promoters of the present invention were found to provide higher expression levels and/or improved transduction efficiency of CNS cells as compared to constructs comprising a minimal promoter. Moreover, MeCP2 promoters comprising a synthetic intron were found to provide the highest expression levels and transduction efficiency.

Thus, the present invention provides a nucleic acid construct comprising a methyl CpG binding protein 2 (MeCP2) promoter operably linked to a nucleotide sequence encoding a progranulin (PGRN) protein.

The present invention also provide a nucleic acid construct comprising an engineered methyl CpG binding protein 2 (MeCP2) promoter operably linked to a nucleotide sequence encoding a protein of interest (POI), wherein the engineered MeCP2 promoter comprises a minimal promoter sequence and at least one intron.

The present invention further provides a vector comprising a nucleic acid construct of the invention. The vector may be a plasmid or a viral vector.

The present invention further provides a host cell which comprises a nucleic acid construct of the invention and/or a vector of the invention, and/or which produces a viral vector of the invention, optionally wherein the host cell is a HEK293 cell or a HEK293T cell.

Further provided by the present invention is a pharmaceutical composition comprising a nucleic acid construct of the invention, a vector of the invention, and/or a viral vector of the invention together with a pharmaceutically acceptable carrier, excipient or diluent.

Also provided by the present invention is a nucleic acid construct of the invention, a vector of the invention, a viral vector of the invention, and/or a pharmaceutical composition of the invention for use in a method of treating or preventing a disease characterized by progranulin (PGRN) deficiency in a patient in need thereof.

The present invention further provides a method of treating or preventing a disease characterized by progranulin (PGRN) deficiency in a patient in need thereof, said method comprising administering to the patient a therapeutically effective amount of a nucleic acid construct of the invention, a vector of the invention, a viral vector of the invention, and/or a pharmaceutical composition of the invention.

The present invention also provides the use of a nucleic acid construct of the invention, a vector of the invention, a viral vector of the invention, and/or a pharmaceutical composition of the invention for the manufacture of a medicament for the treatment or prevention of a disease characterized by progranulin (PGRN) deficiency in a patient in need thereof.

### BRIEF DESCRIPTION OF THE FIGURES

FIG. 1. A. Schematics showing organization of constructs pAK169, pPG21, pPG35 and pPG36. MeCP2 (250 bp) denotes a minimal MeCP2 promoter sequence. GFP denotes a gene encoding green fluorescent protein. 5′ MeCP2 (2100 bp) denotes a natural intron of approximately 2100 bp 5′ to MeCP2 (250 bp). PGRN (1800 bp) denotes a polynucleotide sequence encoding PGRN. Intron (2100 bp) denotes a synthetic intron sequence of approximately 100 bp in length. B. Image of western blot analysis of promoter activity. PGRN expression was evaluated for each of pAK169, pPG21, pPG35 and pPG36.

FIG. 2. A. Schematic showing organization pAK168, pPG20, pPG33 and pPG34. NSE1 (1300 bp) denotes a minimal NSE1 promoter sequence. GFP denotes a gene encoding green fluorescent protein. 5′ NSE1 (1100 bp) denotes a natural intron of approximately 1100 bp 5′ to NSE1 (250 bp). PGRN (1800 bp) denotes a polynucleotide sequence encoding PGRN. Intron (900 bp) denotes a synthetic intron sequence of approximately 900 bp in length. B. Image of western blot analysis of promoter activity. PGRN expression was evaluated for each of pAK168, pPG20, pPG33 and pPG34.

FIG. 3. Evaluation of PGRN expression in primary neurons and astrocytes for constructs pPG20, pPG33, pPG34, pPG21, pPG21, pPG35 and pPG36. A. Bar charts showing: (A) transduction efficiency in neurons; (B) PGRN expression levels in transduced neurons; (C) transduction efficiency in astrocytes; and (D) PGRN expression levels in transduced astrocytes.

FIG. 4. Evaluation of PGRN secretion by primary neurons and astrocytes. Bar chart showing concentration of PGRN secreted by neuron-astrocyte co-cultures transduced with constructs pPG21, pPG35, pPG36, pPG20, pPG26. An untransduced control is also shown.

FIG. 5. Codon-optimization of nucleic acid constructs encoding PGRN. A. Bar chart showing expression levels of PGRN for GRN^{-/-} HAP-1 cells transfected with PGRN-encoding lentiviral vectors, as determined by ELISA. Vectors comprising a codon-optimized nucleotide sequence encoding PGRN (denoted CpG 0, 4, 9, 17, 25, 40, 71 and 90) were compared to a vector comprising a wild type nucleotide sequence encoding PGRN (denoted WT). PGRN expression levels for a control transfection with empty vector and WT HAP-1 cells (GRN^{+/+}) are also indicated. B. Image of western blot analysis of PGRN expression levels in GRN^{-/-} HAP-1 cells transfected with a lentiviral vector comprising codon-optimized nucleotide sequences encoding PGRN (denoted CpG 25, 40, 71 and 90) and a vector comprising a wild type nucleotide sequence encoding PGRN (denoted WT). PGRN expression levels are also indicated for a control transfection with empty vector (denoted Mock), untransfected wild type GRN^{+/+} HAP-1 cells (denoted WT), and untransfected GRN^{-/-} HAP-1 cells (denoted KO).

FIG. 6. Expression of human PGRN corrects lysosomal deficit in GRN^{-/-} mouse primary neurons. A. Image of western blot analysis performed to quantify the level of lysosomal protein cathepsin D in WT (GRN^{+/+}) and KO (GRN^{-/-}) primary neurons transduced with a lentiviral vector comprising the pPG36 construct. B. Bar chart showing levels of cathepsin D proteins (immature, mature heavy chain and mature light chain, respectively). Values for expression of cathepsin D are normalized to actin and GADPH expression level.

FIG. 7. ELISA and FRET analysis of CNS expression of human PGRN (hPGRN) in WT and GRN^{-/-} mice following striatal injection of AAVTT-p1PG36. A. Bar chart showing CSF and plasma levels of hPGRN (ng/ml) as measured by ELISA. A high level of hPGRN was detected in the CSF (1:100 dilution) of both WT and GRN^{-/-} mice in animals injected with AAVTT-p1PG36 (an AAVTT vector comprising the pPG36 construct). hPGRN was also detected in the plasma of the mice (1:10 dilution). B. Bar chart showing the results of FRET measurement of hPGRN concentration (ng/mg) in various brain regions of WT or GRN^{-/-} mice injected with AAVTT-p1PG36. Highest expression of hPGRN was detected close to the site of injection (Striatum and Mid-brain). Mid-levels of hPGRN expression was also detected in cortex and hippocampus. Low-levels of hPGRN expression were detected in distant brain regions such as brain-stem, olfactory bulb and cerebellum. C. Bar chart showing CSF levels of hPGRN (ng/ml) as measured by ELISA following WT mice striatal injection of AAVTT-p1PG36 and AAVTT-p2PG36. A high level of hPGRN was detected in CSF (1:100 dilution) in animals injected with both AAV constructs.

FIG. 8. Images of IHC analysis of CNS expression of human PGRN (hPGRN) in GRN^{-/-} mice following striatal injection of AAVTT-p1PG36. IHC staining of hPGRN was observed in brain of GRN^{-/-} KO mice that received striatal administration of AAVTT-p1PG36. The immunoreactive signal was specific to human progranulin since no signal was observed in mice that received vehicle or control AAV-GFP. High levels of hPGRN were detected mainly throughout the forebrain notably in the striatum, thalamus, hypothalamus, cerebral cortex and hippocampus, and in the midbrain in the substantia nigra of GRN^{-/-} KO mice.

FIG. 9. Human PGRN expression impacts cathepsin D activity in vivo. Bar Chart showing measurement of cathepsin D enzymatic activity the mid-brain lysate of WT (GRN^{+/+}) mice treated with vehicle (as indicated by closed circles) and GRN^{-/-} KO mice treated with vehicle (closed circles) or AAVTT-p1PG36 (closed triangles). An increase in cathepsin D enzymatic activity was observed in 4-month old GRN^{-/-} mice. A decrease in cathepsin D activity was observed in GRN^{-/-} mice injected with AAVTT-p1PG36 as compared to mice injected with vehicle.

FIG. 10. Schematic showing the organization of component nucleic acid sequences in the AAVTT-pPG36 construct (SEQ ID NO: 17).

FIG. 11. Schematic showing the location of the component regions of the MeCP2_2 intron (SEQ ID NO: 2) within the full length murine MECP2 gene.

### BRIEF DESCRIPTION OF THE SEQUENCES

SEQ ID NO: 1 is the nucleotide sequence of the MeCP2 minimal promoter.

SEQ ID NO: 2 is the nucleotide sequence of the MeCP2_2 intron.

SEQ ID NO: 3 is the nucleotide sequence of the MeCP2_2 promoter.

SEQ ID NO: 4 is the nucleotide sequence of exon1 of the MeCP2_2 intron.

SEQ ID NO: 5 is the nucleotide sequence of the 5′ intron of the MeCP2_2 intron.

SEQ ID NO: 6 is the nucleotide sequence of the 3′ intron of the MeCP2_2 intron.

SEQ ID NO: 7 is nucleotide sequence of exon2 of the MeCP2_2 intron.

SEQ ID NO: 8 is the nucleotide sequence of the MeCP2_1 promoter.

SEQ ID NO: 9 is the nucleotide sequence of the MeCP2_1 intron.

SEQ ID NOs: 10 and 11 are the nucleotide sequences of constructs pPG35 and pPG36, respectively.

SEQ ID NOs: 12 and 13 correspond to human PGRN nucleotide and amino acid sequences, respectively.

SEQ ID NO: 14 is the nucleotide sequence of the Age1 restriction site (5′-ACCGGT-3′).

SEQ ID NO: 15 is the nucleotide sequence of a woodchuck hepatitis virus (WHP) posttranscriptional regulatory element (WPRE).

SEQ ID NO: 16 is the nucleotide sequence of an SV40 polyadenylation (poly(A) signal) sequence.

SEQ ID NO: 17 is the nucleotide sequence of the AAVTT-pPG36 construct.

SEQ ID NO: 18 is the nucleotide sequence of the AAVTT-p1PG36 plasmid.

SEQ ID NO: 19 is the nucleotide sequence of the AAVTT-p2PG36 plasmid.

SEQ ID NO: 20 is the nucleotide sequence of the 5′ ITR as used in the AAVTT-pPG36 construct.

SEQ ID NO: 21 is the nucleotide sequence of the 5′ adjacent fragment as used in the AAVTT-pPG36 construct.

SEQ ID NO: 22 is the nucleotide sequence of the 3′ adjacent fragment as used in the AAVTT-pPG36 construct.

SEQ ID NO: 23 is the nucleotide sequence of the 3′ ITR as used in the AAVTT-pPG36 construct.

SEQ ID NO: 24 is the nucleotide sequence of the Kozak sequence as used in the AAVTT-pPG36 construct.

### DETAILED DESCRIPTION OF THE INVENTION

All publications, patents and patent applications cited herein, whether supra or infra, are incorporated by reference in their entirety.

### Definitions

As used in this specification and the appended claims, the singular forms “a”, “an”, and “the” include plural referents unless the content clearly dictates otherwise. Thus, for example, reference to “a nucleic acid” includes “nucleic acids”, and the like.

The term “comprises” (comprise, comprising) should be understood to have its normal meaning in the art, i.e. that the stated feature or group of features is included, but that the term does not exclude any other stated feature or group of features from also being present. For example, a promoter comprising a minimal promoter sequence may contain other components, such as one or more introns. The term “consists of” should also be understood to have its normal meaning in the art, i.e. that the stated feature or group of features is included, to the exclusion of further features. For example, a promoter consisting of a minimal promoter sequence contains the minimal promoter sequence and no other components. For every embodiment in which “comprises” or “comprising” is used, we anticipate a further embodiment in which “consists of” or “consisting of” is used. Thus, every disclosure of “comprises” should be considered to be a disclosure of “consists of”.

The terms “protein” and “polypeptide” are used interchangeably herein and, in their broadest sense, refer to a compound of two or more subunit amino acids, amino acid analogs, or other peptidomimetics. The terms “protein” thus includes short peptide sequences and also longer polypeptides. As used herein, the term “amino acid” refers to either natural and/or unnatural or synthetic amino acids, including both D or L optical isomers, and amino acid analogs and peptidomimetics.

The terms “patient” and “subject” are used interchangeably herein. Typically, the patient is a human.

Sequence Homology/Identity

Although sequence homology can also be considered in terms of functional similarity (i.e., amino acid residues having similar chemical properties/functions), in the context of the present document it is preferred to express homology in terms of sequence identity.

Sequence comparisons can be conducted by eye or, more usually, with the aid of readily available sequence comparison programs. These publicly and commercially available computer programs can calculate percent homology (such as percent identity) between two or more sequences.

Percent identity may be calculated over contiguous sequences, i.e., one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues (for example less than 50 contiguous amino acids). For comparison over longer sequences, gap scoring is used to produce an optimal alignment to accurately reflect identity levels in related sequences having insertion(s) or deletion(s) relative to one another. A suitable computer program for carrying out such an alignment is the GCG Wisconsin Bestfit package (University of Wisconsin, U.S.A; Devereux et al., 1984, Nucleic Acids Research 12:387). Examples of other software than can perform sequence comparisons include, but are not limited to, the BLAST package, FASTA (Altschul et al., 1990, J. Mol. Biol. 215:403-410) and the GENEWORKS suite of comparison tools.

Typically sequence comparisons are carried out over the length of the reference sequence. For example, if the user wished to determine whether a given sequence is 70% identical to SEQ ID NO: 2, SEQ ID NO: 2 would be the reference sequence. For example, to assess whether a sequence is at least 90% identical to SEQ ID NO: 2 (an example of a reference sequence), the skilled person would carry out an alignment over the length of SEQ ID NO: 2, and identify how many positions in the test sequence were identical to those of SEQ ID NO: 2. If at least 70% of the positions are identical, the test sequence is at least 70% identical to SEQ ID NO: 2. If the sequence is shorter than SEQ ID NO: 27, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm. In an embodiment, the percent identity between two amino acid or nucleic acid sequences is determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6.

The term “fragment” as used herein refers to a contiguous portion of a reference sequence. For example, a fragment of SEQ ID NO: 2 of 50 nucleotides in length refers to 50 contiguous nucleotides of SEQ ID NO: 2.

The term “functional variant” as used herein refers to a nucleic acid or amino acid sequence which has been modified relative to a reference sequence but which retains the function of said reference sequence. For example, a functional variant of an MeCP2 promoter retains the ability to drive expression of a nucleotide sequence encoding a POI in cells of the CNS, such as neurons or astrocytes. Similarly, a functional variant of a PGRN protein retains the activities of the reference PGRN protein.

Nucleic Acids

The terms “polynucleotide” and “nucleic acid molecule” are used interchangeably herein and refer to a polymeric form of nucleotides of any length, either deoxyribonucleotides or ribonucleotides, or analogs thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A polynucleotide of the invention may be provided in isolated or substantially isolated form. By substantially isolated, it is meant that there may be substantial, but not total, isolation of the polypeptide from any surrounding medium. The polynucleotides may be mixed with carriers or diluents which will not interfere with their intended use and still be regarded as substantially isolated. A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences, for example in an expression vector. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

Polynucleotides can be synthesised according to methods well known in the art, as described by way of example in Sambrook et al (1989, Molecular Cloning—a laboratory manual; Cold Spring Harbor Press).

The term “nucleic acid construct” as used herein refers to an artificial (e.g. recombinantly produced or synthesized) nucleic acid comprising at least one control sequence (such as a promoter) and at least one nucleotide sequence encoding a protein of interest (POI). Thus, a nucleic acid construct of the present invention may be considered an expression cassette. The nucleic acid constructs of the present invention may be isolated, or substantially isolated. Typically, the nucleic acid constructs of the present invention comprise a control sequence (such as an MeCP2 promoter) operably linked to a nucleotide sequence encoding a protein of interest (such as PGRN), thus allowing for expression of the protein of interest in vivo. Nucleic acid constructs of the present invention may comprise appropriate promoters, enhancers, initiators, and other elements, such as for example polyadenylation (polyA) signals and/or a woodchuck hepatitis virus posttranscriptional regulatory element (WPRE) sequence. Nucleic acid constructs of the present invention may also comprise nucleotide sequences which facilitate their genetic manipulation, such as restriction sites (e.g. an Age1 restriction site having the nucleotide sequence of SEQ ID NO: 14).

As used herein the term “operably linked” refers to a juxtaposition of two or more nucleotide sequences which allows each of said two or more sequences to perform their normal function. Typically, the term operably linked is used to refer to the juxtaposition of a regulatory element (e.g. a promoter, enhancer, polyA signal sequence, WPRE sequence, etc.) and a nucleotide sequence encoding a protein of interest (POI). For example, an operable linkage between a promoter and a protein-encoding nucleotide sequence permits the promoter to function to drive the expression of the POI in vivo.

In addition to an MeCP2 promoter, the nucleic acid constructs of the present invention may comprise one or more further regulatory elements. Preferred regulatory elements are those which function to stabilize an mRNA transcribed from the nucleic acid construct and/or enhance the expression of a protein of interest (POI) from the nucleic acid construct, such as PGRN.

A preferred regulatory element which may be used in the nucleic acid constructs of the present invention is a woodchuck hepatitis virus (WHP) posttranscriptional regulatory element (WPRE). A WPRE is a DNA sequence which, when transcribed into mRNA, creates tertiary structure in the mRNA transcript thereby enhancing the stability of the mRNA and expression of the POI encoded by the nucleic acid construct. In the nucleic acid constructs of the present invention, the WPRE may be 3′ to the nucleotide sequence encoding the POI or the PGRN protein. The WPRE may comprise the nucleotide sequence of SEQ ID NO: 15 or a functional variant or fragment thereof having at least 90%, 91%, 92%, 93%, 94%, 95%, 96%, 97%, 98%, 99%, 99.5%, or 99.9% identity to the nucleotide sequence of SEQ ID NO: 15. The functional variant or fragment of the WPRE retains the characteristics of the corresponding non-variant or full-length WPRE. Thus, the variant or fragment WPRE may be capable of creating tertiary structure in an mRNA transcript and/or enhancing the stability of an mRNA transcript and/or enhancing expression of the POI encoded by the nucleic acid construct. The enhancement is relative to an mRNA not containing the variant or fragment WPRE.

A preferred regulatory element which may be used in the nucleic acid constructs of the present invention is a polyadenylation (poly(A)) signal sequence. In eukaryotic cells, polyadenylation signal sequences within mRNA transcripts are recognized and processed to add a poly(A) tail consisting of multiple adenosine monophosphates at the 3′ end of the mRNA transcript. The poly(A) tail functions to promote export of the mRNA from the nucleus to the cytoplasm and prevents the degradation of the mRNA, thereby enhancing expressing of the POI encoded by the nucleic acid construct. In the nucleic acid constructs of the present invention, the polyadenylation signal sequence may be 3′ to the nucleotide sequence encoding the POI or the PGRN protein. The polyadenylation signal sequence may comprise the nucleotide sequence SEQ ID NO: 16 or a functional variant or fragment thereof having at least 90% identity to the nucleotide sequence of SEQ ID NO: 16. The functional variant or fragment of the polyadenylation sequence retains the characteristics of the corresponding non-variant or full-length polyadenylation signal sequence.

The nucleic acid constructs of the present invention may comprise, in the 5′ to 3′ direction, an MeCP2 promoter, a nucleotide sequence encoding the POI or the PGRN protein, an WPRE, and a polyadenylation signal sequence.

The nucleic acid constructs of the present invention may be provided within vectors (e.g., plasmids or recombinant viral vectors). A suitable vector may be any vector which is capable of carrying a sufficient amount of genetic information and allowing expression of a POI in vivo. A vector comprising a nucleic acid construct of the invention may be administered directly to a patient in need thereof. Such vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. Other suitable vectors would be apparent to persons skilled in the art. By way of further example in this regard we refer to Sambrook et al. (1989, Molecular Cloning—a laboratory manual; Cold Spring Harbor Press).

Methyl CpG Binding Protein 2 (MeCP2) Promoters

Methyl CpG binding protein 2 (MeCP2) is a transcriptional repressor and has been proposed to globally silence the transcription of genes by binding to methylated cytosine nucleotides within the promoters of these genes and subsequently recruiting the co-repressor protein complexes. Additionally, MeCP2 binds to DNA methyltransferase 1 as well as regulates histone methyltransferase activity, which serves to maintain DNA methylation and facilitates methylation of Lys9 in histone H3. Thus, by binding to methylated DNA, MeCP2 reinforces its repressive function through multiple epigenetic modifications, such as the maintenance of DNA methylation and the deacetylation and methylation of histones.

MeCP2 is highly expressed in the brain, lung and spleen and moderately in the heart and kidney. In particular, within the central nervous system (CNS), MeCP2 is expressed in high concentrations in neurons.

The human MECP2 gene (Gene ID: 4204) is approximately 122 kbp in length, is located on the long arm of the X chromosome (Xq28) and comprises four coding exons (Singh et al. Nucleic Acids Research. (2008) Vol. 36, No. 19 6035-6047). The murine MECP2 gene (Gene ID: 17257) is approximately 59 kbp in length and is located on the murine X chromosome at position ChrX:73070198-73129296 bp (−strand).

Two MeCP2 isoforms have been identified: MeCP2_e1 (e1) and MeCP2_e2 (e2). The e1 isoform is 498 amino acids in length and encoded by exons 1, 3 and 4. The e2 isoform is 486 amino acids long and encoded by exons 2, 3 and 4. The promoter regions of the murine and human MECP2 genes have been characterised by, inter alia, Adachi et al. (Hum. Mol. Genetics. 2005; 14(23): 3709-3722). A segment of the MIECP2 gene (−677/+56) was found to exhibit strong promoter activity in neuronal cell lines and cortical neurons, but was inactive in non-neuronal cells and glia. The region necessary for neuronal-specific promoter activity (termed the MR element) was observed to be located within a 19 bp region (−63/−45).

As described in Adachi et al. (Hum. Mol. Genetics. 2005; 14(23): 3709-3722), the sequence of the murine (−677/+56) region of the MECP2 gene is 68% similar to the corresponding human MeCP2 promoter. In particular, the human and murine sequences are 92% identical between nucleotide positions −87 and +56, which contains the MR element.

The MeCP2 sequences described herein (e.g. SEQ ID NOs: 1-9) and used in the exemplified constructs are derived from the murine MECP2 gene. However, as noted above, there is a high level of sequence similarity between the minimal promoter regions of the murine and human MECP2 genes. Furthermore, there is a very high degree of sequence identity between the murine and human MR element, which is responsible for neuronal specific expression. Accordingly, for each embodiment of the present invention which comprises one or more murine MeCP2 nucleotide sequences, there is also provided an embodiment in which said one or more murine MeCP2 nucleotide sequences has been replaced by a corresponding human MeCP2 nucleotide sequence.

Thus, as used herein the term “MeCP2 promoter” refers to a nucleotide sequence of an MECP2 gene (e.g. a murine or a human MECP2 gene) which is capable of functioning as promoter, i.e. capable of driving the transcription of a nucleotide sequence to which said MeCP2 promoter is operably linked, thereby driving the expression of a protein encoded by said nucleotide sequence. Typically, an MeCP2 promoter sequence as used in the present invention will be specific for a particular tissue or cell type(s). Preferably, an MeCP2 promoter used in the present invention will be specific for cells of the CNS. More preferably, an MeCP2 promoter used in the present invention will specifically drive expression of a protein of interest (POI), such as PGRN, in the neurons and/or the astrocytes.

The MeCP2 promoter used in the nucleic acid constructs of the present invention may be a functional variant or fragment of an MeCP2 promoter described herein. A functional variant or fragment of an MeCP2 promoter described herein may be functional in the sense that it retains the characteristics of the corresponding non-variant or full-length MeCP2 promoter. Thus, a functional variant or fragment of an MeCP2 promoter described herein retains the capacity to drive the transcription of a nucleotide sequence to which said functional variant or fragment is operably linked, thereby driving the expression of a protein encoded by said nucleotide sequence. A functional variant or fragment of an MeCP2 promoter described herein may retain specificity for a particular tissue type. For example, a functional variant or fragment of an MeCP2 promoter described herein may be specific for cells of the CNS. A functional variant or fragment of an MeCP2 promoter described herein may specifically drive expression of a protein of interest (POI), such as PGRN, in the neurons and/or the astrocytes.

An MeCP2 promoter used in the present invention may comprise a “minimal promoter sequence”, which should be understood to be a nucleotide sequence of the promoter region of an MECP2 gene of sufficient length and which comprise the required elements to function as an MeCP2 promoter, i.e. capable of driving the transcription of a nucleotide sequence to which said MeCP2 promoter is operably linked, thereby driving the expression of a protein encoded by said nucleotide sequence.

The minimal MeCP2 promoter used in the nucleic acid constructs of the present invention may be a functional variant or fragment of a minimal MeCP2 promoter described herein. A functional variant or fragment of a minimal MeCP2 promoter described herein may be functional in the sense that it retains the characteristics of the corresponding non-variant or full-length minimal MeCP2 promoter. Thus, a functional variant or fragment of a minimal MeCP2 promoter described herein is of sufficient length and comprises the required elements to function as an MeCP2 promoter and is capable of driving the transcription of a nucleotide sequence to which said functional variant or fragment is operably linked, thereby driving the expression of a protein encoded by said nucleotide sequence.

A preferred minimal promoter sequence that may be used in the MeCP2 promoters described herein may comprise or consist of the nucleotide sequence of SEQ ID NO: 1 or a functional variant thereof having at least 90%, 91%, 92%, 93%, 94%, 95%, 96%, 97%, 98%, 99%, 99.5%, or 99.9% identity to the nucleotide sequence of SEQ ID NO: 1. Fragments of any length of SEQ ID NO: 1 or said functional variant may also be used as a minimal promoter sequence in the nucleic acid constructs of the present invention. The minimal promoter sequence may be 160-300 bp, 170-290 bp, 180-280 bp, 190-270 bp, 200-260 bp, 210-250 bp, 220-240 bp, or about 230 bp.

An MeCP2 promoter used in the present invention may comprise one or more introns. As used herein, the term “intron” refers to an intragenic non-coding nucleotide sequence. Typically, introns are transcribed from the DNA into messenger RNA (mRNA) during transcription of a gene but are excised from the mRNA transcript by splicing prior to its translation.

An MeCP2 promoter used in the present invention may comprise a functional variant or fragment of an intron described herein. A functional variant or fragment of an intron described herein may be functional in the sense that it retains the characteristics of the corresponding non-variant or full-length intron. Thus, functional variants or fragments of an intron described herein are non-coding. Functional variants or fragments of an intron described herein may also retain the capacity to be transcribed from DNA to mRNA and/or the capacity to be excised from mRNA by splicing.

An MeCP2 promoter which comprises a minimal promoter sequence and an intron is referred to herein as an “engineered MeCP2 promoter”.

Introns that may be incorporated in the MeCP2 promoters used in the present invention may be from naturally non-coding region of an MECP2 gene. Thus, the term intron encompasses a nucleotide sequence corresponding to a naturally occurring contiguous nucleotide sequence of an MECP2 gene. Such introns are referred to herein as “natural” introns.

A preferred intron that may be used in the MeCP2 promoters described herein comprises or consists of the nucleotide sequence of SEQ ID NO: 9 or a functional variant thereof having at least 90%, 91%, 92%, 93%, 94%, 95%, 96%, 97%, 98%, 99%, 99.5%, or 99.9% identity to SEQ ID NO: 9. Fragments of said intron may also be used. Such fragments may be 1000-2107 bp, 1200-2100 bp, 1400-2000 bp, 1600-1900 bp, or 1700-1800 bp in length. Longer nucleotide sequences comprising said intron may also be used.

A preferred MeCP2 promoter that may be used in the nucleic acid constructs of the present invention is designated MeCP2_1 (SEQ ID NO: 8). This MeCP2 promoter comprises an intron having the nucleotide sequence of SEQ ID NO: 9. Thus, the MeCP2 promoter used in the nucleic acid construct of the present invention may comprise or consist of the nucleotide sequence of SEQ ID NO: 8 or a functional variant thereof having at least 90%, 91%, 92%, 93%, 94%, 95%, 96%, 97%, 98%, 99%, 99.5%, or 99.9% identity to SEQ ID NO: 8. Fragments of said MeCP2 promoter may also be used. Such fragments may be 1000-2336 bp, 1200-2300 bp, 1400-2200 bp, 1600-2100 bp, 1700-2000 bp, or 1800-1900 bp in length. Longer nucleotide sequences comprising said MeCP2 promoter may also be used.

The MeCP2 promoters used in the nucleic acid constructs of the present invention may comprise a “synthetic intron”. A synthetic intron should be understood as one which has been constructed from two or more disparate (e.g., distinct and non-contiguous) sequences, for example of an MECP2 gene. The two or more sequences used to prepare the synthetic intron can be from any location with an MECP2 gene. The synthetic intron may therefore comprise a nucleotide sequence of an intron of an MECP2 gene, which is thus termed an “intronic sequence”.

Alternatively, the component nucleotide sequences of the synthetic intron need not be from an intron of an MECP2 gene and may instead be from an exon (i.e. the protein encoding nucleotides) of the MECP2 gene. Typically, the nucleotide sequence of an exon MECP2 gene will be modified (e.g., by truncation, deletion, substitution, and the like) and/or arranged within the synthetic intron such that the exonic sequence is non-expressing. Thus, such nucleotide sequences are not capable of producing a transcript which may be translated into polypeptide (e.g., an MeCP2 protein or a fragment thereof). Accordingly, the synthetic introns used in the MeCP2 promoters described herein may comprise one or more “non-expressing exonic sequences”, for example of an MECP2 gene. Suitably, said non-expressing exonic sequences may flank an intronic sequence to provide splice sites. These splice sites allow the synthetic intron to be excised by splicing from an mRNA produced by transcription of a nucleic acid construct comprising said synthetic intron.

A synthetic intron used in the MeCP2 promoters described herein may comprise a functional variant or fragment of a non-expressing exonic sequence described herein. A functional variant or fragment of a non-expressing exonic sequence described herein may be functional in the sense that it retains the characteristics of the corresponding non-variant or full-length exonic sequence. Thus, functional variants or fragments of the non-expressing exonic sequences described herein may retain the capacity to flank an intronic sequence and may comprise splice sites. They may be capable of being joined (or spliced) together upon removal of the exon.

The synthetic intron used in the MeCP2 promoters described herein may comprise one, two, three, four, five, six, seven, eight, nine, or ten intronic sequences and/or one, two, three, four, five, six, seven, eight, nine, or ten non-expressing exonic sequences. Preferably, the synthetic intron comprises two intronic sequences and two non-expressing exonic sequences.

A preferred non-expressing exonic sequence that may be used in the MeCP2 promoters described herein comprises or consists of the nucleotide sequence of SEQ ID NO: 4 or a functional variant thereof having at least 90%, 91%, 92%, 93%, 94%, 95%, 96%, 97%, 98%, 99%, 99.5%, or 99.9% identity to SEQ ID NO: 4. Fragments of said non-expressing exonic sequences may also be used. Longer nucleotide sequences comprising said non-expressing exonic sequence may also be used.

A preferred non-expressing exonic sequence that may be used in the MeCP2 promoters described herein comprise or consists of the nucleotide sequence of SEQ ID NO: 7 or a functional variant thereof having at least 90%, 91%, 92%, 93%, 94%, 95%, 96%, 97%, 98%, 99%, 99.5%, or 99.9% identity to SEQ ID NO: 7. Fragments of said non-expressing exonic sequences may also be used. Longer nucleotide sequences comprising said non-expressing exonic sequence may also be used.

A preferred intronic sequence that may be used in the MeCP2 promoters described herein comprises or consists of the nucleotide sequence of SEQ ID NO: 5 or a functional variant thereof having at least 90%, 91%, 92%, 93%, 94%, 95%, 96%, 97%, 98%, 99%, 99.5%, or 99.9% identity to SEQ ID NO: 5. Fragments of said intronic sequences may also be used. Longer nucleotide sequences comprising said intronic sequences may also be used.

A preferred intronic sequence that may be used in the MeCP2 promoters described herein comprises or consists of the nucleotide sequence of SEQ ID NO: 6 or a functional variant thereof having at least 90%, 91%, 92%, 93%, 94%, 95%, 96%, 97%, 98%, 99%, 99.5%, or 99.9% identity to SEQ ID NO: 6. Fragments of said intronic sequences may also be used. Longer nucleotide sequences comprising said intronic sequences may also be used.

Thus, the nucleic acid construct of the invention may comprise or consist of an MeCP2 promoter comprising at least one synthetic intron which comprises:
(a) a non-expressing exonic sequence comprising the nucleotide sequence of SEQ ID NO: 4 or a functional variant or fragment thereof having at least 90%, 91%, 92%, 93%, 94%, 95%, 96%, 97%, 98%, 99%, 99.5%, or 99.9% identity to SEQ ID NO: 4;
(b) an intronic sequence comprising a nucleotide sequence of SEQ ID NO: 5 or a functional variant or fragment thereof having at least 90%, 91%, 92%, 93%, 94%, 95%, 96%, 97%, 98%, 99%, 99.5%, or 99.9% identity to SEQ ID NO: 5;
(c) an intronic sequence comprising a nucleotide sequence of SEQ ID NO: 6 or a functional variant or fragment thereof having at least 90%, 91%, 92%, 93%, 94%, 95%, 96%, 97%, 98%, 99%, 99.5%, or 99.9% identity to SEQ ID NO: 6; and/or
(d) a non-expressing exonic sequence comprising the nucleotide sequence of SEQ ID NO: 7 or a functional variant or fragment thereof having at least 90%, 91%, 92%, 93%, 94%, 95%, 96%, 97%, 98%, 99%, 99.5%, or 99.9% identity to SEQ ID NO: 7.

The synthetic intron may comprise (a), (b), (c) and/or (d) above in any order in the 5′ to 3′ direction. The synthetic intron may comprise (a), (b), (c) and/or (d) in the order that they are listed above. For example, in the 5′ to 3′ direction, the synthetic intron may comprise:
i. (a) and (b);
ii. (a) and (c);
iii. (a) and (d);
iv. (b) and (c);
v. (b) and (d);
vi. (c) and (d);
vii. (a), (b) and (c);
viii. (a), (b) and (d);
ix. (b), (c) and (d); or
x. (a), (b), (c) and (d).

The synthetic intron may comprise a non-expressing exonic sequence at its 5′ terminus.

For example, the synthetic intron may comprise at its 5′ terminus:
(a) a non-expressing exonic sequence comprising the nucleotide sequence of SEQ ID NO: 4 or a functional variant or fragment thereof having at least 90%, 91%, 92%, 93%, 94%, 95%, 96%, 97%, 98%, 99%, 99.5%, or 99.9% identity to SEQ ID NO: 4; or
(d) a non-expressing exonic sequence comprising the nucleotide sequence of SEQ ID NO: 7 or a functional variant or fragment thereof having at least 90%, 91%, 92%, 93%, 94%, 95%, 96%, 97%, 98%, 99%, 99.5%, or 99.9% identity to SEQ ID NO: 7.

The synthetic intron may comprise a non-expressing exonic sequence at its 3′ terminus:
(a) a non-expressing exonic sequence comprising the nucleotide sequence of SEQ ID NO: 4 or a functional variant or fragment thereof having at least 90%, 91%, 92%, 93%, 94%, 95%, 96%, 97%, 98%, 99%, 99.5%, or 99.9% identity to SEQ ID NO: 4; or
(d) a non-expressing exonic sequence comprising the nucleotide sequence of SEQ ID NO: 7 or a functional variant or fragment thereof having at least 90%, 91%, 92%, 93%, 94%, 95%, 96%, 97%, 98%, 99%, 99.5%, or 99.9% identity to SEQ ID NO: 7.

The synthetic intron may comprise non-expressing exonic sequences at its 5′ terminus and at its 3′ terminus. For example, the synthetic intron may comprise at its 5′ terminus:
(a) a non-expressing exonic sequence comprising the nucleotide sequence of SEQ ID NO: 4 or a functional variant or fragment thereof having at least 90%, 91%, 92%, 93%, 94%, 95%, 96%, 97%, 98%, 99%, 99.5%, or 99.9% identity to SEQ ID NO: 4; or
(d) a non-expressing exonic sequence comprising the nucleotide sequence of SEQ ID NO: 7 or a functional variant or fragment thereof having at least 90%, 91%, 92%, 93%, 94%, 95%, 96%, 97%, 98%, 99%, 99.5%, or 99.9% identity to SEQ ID NO: 7; and the synthetic intron may comprise at its 3′ terminus:
(a) a non-expressing exonic sequence comprising the nucleotide sequence of SEQ ID NO: 4 or a functional variant or fragment thereof having at least 90%, 91%, 92%, 93%, 94%, 95%, 96%, 97%, 98%, 99%, 99.5%, or 99.9% identity to SEQ ID NO: 4; or
(d) a non-expressing exonic sequence comprising the nucleotide sequence of SEQ ID NO: 7 or a functional variant or fragment thereof having at least 90%, 91%, 92%, 93%, 94%, 95%, 96%, 97%, 98%, 99%, 99.5%, or 99.9% identity to SEQ ID NO: 7.

The non-expressing exonic sequences at the 5′ terminus and the 3′ terminus may flank one or more intronic sequences, such as one or more intronic sequence described herein. For example, in the 5′ to 3′ direction, a synthetic intron used in the MeCP2 promoters described herein may comprise:
i. (a), (b) and (d);
ii. (a), (c) and (d);
iii. (a), (b), (c) and (d);
iv. (a), (c), (b) and (d);
v. (a), (b) and (a);
vi. (a), (c) and (a);
vii. (a), (b), (c) and (a);
viii. (a), (c), (b) and (a)
ix. (d), (b) and (d);
x. (d), (c) and (d);
xi. (d), (b), (c) and (d);
xii. (d), (c), (b) and (d)
xiii. (d), (b), and (a);
xiv. (d), (c) and (a);
xv. (d), (b), (c) and (a); or
xvi. (d), (c), (d) and (a), where:
   (a) corresponds to a non-expressing exonic sequence comprising the nucleotide sequence of SEQ ID NO: 4 or a functional variant or fragment thereof having at least 90%, 91%, 92%, 93%, 94%, 95%, 96%, 97%, 98%, 99%, 99.5%, or 99.9% identity to SEQ ID NO: 4;
   (b) corresponds to an intronic sequence comprising a nucleotide sequence of SEQ ID NO: 5 or a functional variant or fragment thereof having at least 90%, 91%, 92%, 93%, 94%, 95%, 96%, 97%, 98%, 99%, 99.5%, or 99.9% identity to SEQ ID NO: 5;
   (c) corresponds to an intronic sequence comprising a nucleotide sequence of SEQ ID NO: 6 or a functional variant or fragment thereof having at least 90%, 91%, 92%, 93%, 94%, 95%, 96%, 97%, 98%, 99%, 99.5%, or 99.9% identity to SEQ ID NO: 6; and
   (d) corresponds to a non-expressing exonic sequence comprising the nucleotide sequence of SEQ ID NO: 7 or a functional variant or fragment thereof having at least 90%, 91%, 92%, 93%, 94%, 95%, 96%, 97%, 98%, 99%, 99.5%, or 99.9% identity to SEQ ID NO: 7.

A preferred synthetic intron that may be used in the MeCP2 promoters described herein comprises or consists of, in the 5′ to 3′ direction:
(a) a non-expressing exonic sequence comprising the nucleotide sequence of SEQ ID NO: 4 or a functional variant or fragment thereof having at least 90%, 91%, 92%, 93%, 94%, 95%, 96%, 97%, 98%, 99%, 99.5%, or 99.9% identity to SEQ ID NO: 4;
(b) an intronic sequence comprising a nucleotide sequence of SEQ ID NO: 5 or a functional variant or fragment thereof having at least 90%, 91%, 92%, 93%, 94%, 95%, 96%, 97%, 98%, 99%, 99.5%, or 99.9% identity to SEQ ID NO: 5;
(c) an intronic sequence comprising a nucleotide sequence of SEQ ID NO: 6 or a functional variant or fragment thereof having at least 90%, 91%, 92%, 93%, 94%, 95%, 96%, 97%, 98%, 99%, 99.5%, or 99.9% identity to SEQ ID NO: 6; and/or
(d) a non-expressing exonic sequence comprising the nucleotide sequence of SEQ ID NO: 7 or a functional variant or fragment thereof having at least 90%, 91%, 92%, 93%, 94%, 95%, 96%, 97%, 98%, 99%, 99.5%, or 99.9% identity to SEQ ID NO: 7.

A preferred synthetic intron that may be used in the MeCP2 promoters described herein comprises or consists of, in the 5′ to 3′ direction:
(a) a non-expressing exonic sequence comprising the nucleotide sequence of SEQ ID NO: 4 or a functional variant or fragment thereof having at least 90%, 91%, 92%, 93%, 94%, 95%, 96%, 97%, 98%, 99%, 99.5%, or 99.9% identity to SEQ ID NO: 4;
(b) an intronic sequence comprising a nucleotide sequence of SEQ ID NO: 5 or a functional variant or fragment thereof having at least 90%, 91%, 92%, 93%, 94%. 95%, 96%, 97%, 98% 99% 99.5%, or 99.9% identity to SEQ ID NO: 5;
(c) an intronic sequence comprising a nucleotide sequence of SEQ ID NO: 6 or a functional variant or fragment thereof having at least 90%, 91%, 92%, 93%, 94%, 95%, 96%, 97%, 98%, 99%, 99.5%, or 99.9% identity to SEQ ID NO: 6; and
(d) a non-expressing exonic sequence comprising the nucleotide sequence of SEQ ID NO: 7 or a functional variant or fragment thereof having at least 90%, 91%, 92%, 93%, 94%, 95%, 96%, 97%, 98%, 99%, 99.5%, or 99.9% identity to SEQ ID NO: 7.

A preferred synthetic intron that may be used in the MeCP2 promoters described herein consists of, in the 5′ to 3′ direction:
(a) a non-expressing exonic sequence comprising the nucleotide sequence of SEQ ID NO: 4 or a functional variant or fragment thereof having at least 90%, 91%, 92%, 93%, 94%, 95%, 96%, 97%, 98%, 99%, 99.5%, or 99.9% identity to SEQ ID NO: 4;
(b) an intronic sequence comprising a nucleotide sequence of SEQ ID NO: 5 or a functional variant or fragment thereof having at least 90%, 91%, 92%, 93%, 94%, 95%, 96%, 97%, 98%, 99%, 99.5%, or 99.9% identity to SEQ ID NO: 5;
(c) an intronic sequence comprising a nucleotide sequence of SEQ ID NO: 6 or a functional variant or fragment thereof having at least 90%, 91%, 92%, 93%, 94%, 95%, 96%, 97%, 98%, 99%, 99.5%, or 99.9% identity to SEQ ID NO: 6; and
(d) a non-expressing exonic sequence comprising the nucleotide sequence of SEQ ID NO: 7 or a functional variant or fragment thereof having at least 90%, 91%, 92%, 93%, 94%, 95%, 96%, 97%, 98%, 99%, 99.5%, or 99.9% identity to SEQ ID NO: 7.

A preferred synthetic intron that may be used in the MeCP2 promoters described herein comprises or consists of the nucleotide sequence of SEQ ID NO: 2 or a functional variant thereof having at least 90%, 91%, 92%, 93%, 94%, 95%, 96%, 97%, 98%, 99%, 99.5%, or 99.9% identity to SEQ ID NO: 2. Fragments of said synthetic intron may also be used. Such fragments may be 1000-2005 bp, 1200-2000 bp, 1400-1900 bp, 1600-1800 bp, or 1700-1800 bp in length. Longer nucleotide sequences comprising said synthetic intron may also be used.

A preferred MeCP2 promoter that may be used in the nucleic acid constructs of the present invention is designated MeCP2_2 (SEQ ID NO: 3). This promoter region comprises a synthetic intron having the nucleotide sequence of SEQ ID NO: 2. Thus, the MeCP2 promoter used in the nucleic acid constructs of the present invention may comprise or consist of the nucleotide sequence of SEQ ID NO: 3 or a functional variant thereof having at least 90%, 91%, 92%, 93%, 94%, 95%, 96%, 97%, 98%, 99%, 99.5%, or 99.9% identity to SEQ ID NO: 3. Fragments of said MeCP2 promoters may also be used. Such fragments may be 1000-2234 bp, 1200-2200 bp, 1400-2100 bp, 1600-2000 bp, or 1700-1900 bp in length. Longer nucleotide sequences comprising said MeCP2 promoter may also be used.

Nucleic acid constructs comprising the MeCP2 promoters described herein provide enhanced expression of the protein of interest (POI) that they encode, for example PGRN. Said constructs also provide enhanced transduction efficiency. Thus, in certain embodiments, expression of a POI or a PGRN protein from a nucleic acid construct of the invention comprising an MeCP2 promoter may be increased relative to a construct lacking an MeCP2 promoter that is otherwise identical. In certain embodiments, the nucleic acid construct of the invention comprising an MeCP2 promoter provides increased transduction efficiency relative to a construct lacking an MeCP2 promoter that is otherwise identical.

Nucleic acid constructs comprising the engineered MeCP2 promoters described herein provide enhanced expression of the protein of interest (POI) that they encode, for example PGRN. Said constructs also provide enhanced transduction efficiency. Thus, in certain embodiments, expression of a POI or a PGRN protein from a nucleic acid construct of the invention comprising an engineered MeCP2 promoter may be increased relative to a construct lacking an engineered MeCP2 promoter, such as an equivalent construct comprising a minimal MeCP2 promoter. In certain embodiments, the nucleic acid construct of the invention comprising an engineered MeCP2 promoter provides increased transduction efficiency relative to a construct lacking an engineered MeCP2 promoter, such as an equivalent construct comprising a minimal MeCP2 promoter.

Nucleic acid constructs which comprise the engineered MeCP2 promoters comprising a synthetic intron described herein provide enhanced expression of the protein of interest (POI) that they encode. Said constructs also provide enhanced transduction efficiency. Thus, in certain embodiments, expression of a POI or a PGRN protein from a nucleic acid construct of the invention which comprises an engineered MeCP2 promoter comprising a synthetic intron may be increased relative to a construct lacking an engineered MeCP2 promoter which comprises a synthetic intron, such as a construct comprising a minimal MeCP2 promoter or a construct which comprises an engineered MeCP2 promoter lacking a synthetic intron. In certain embodiments, the nucleic acid construct of the invention which comprises an engineered MeCP2 promoter comprising a synthetic intron provides increased transduction efficiency relative to a construct lacking an engineered MeCP2 promoter which comprises a synthetic intron, such as a construct comprising a minimal MeCP2 promoter or a construct which comprises an engineered MeCP2 promoter lacking a synthetic intron.

Progranulin (PGRN)

Progranulin (PGRN; also known as granulin-epithelin precursor, proepithelin, prostate cancer (PC) cell derived growth factor and acrogranin) is a secreted glycoprotein that is expressed by many cell types throughout the body. Encoded by a single gene (GRN; Gene ID: 2896) on chromosome 17q21, PGRN is a 593-amino acid, cysteine-rich protein with an estimated molecular weight of 68.5 kDa. It contains 7.5 granulin-like domains, each of which consist of highly conserved tandem repeats of a 12 cysteinyl motif. Proteolytic cleavage of PGRN by extracellular proteases, such as elastase, gives rise to smaller peptide fragments termed granulins or epithelins (e.g. granulin A, granulin B, granulin C, etc.). These fragments range in size from 6 to 25 kDa and have been implicated in a range of biological functions.

PGRN deficiency is strongly associated with the pathogenesis of frontotemporal dementia (FTD), also referred to as frontotemporal lobar dementia. A mutation in one allele of the GRN gene, which encodes the protein progranulin (PGRN), is associated with the development of FTD (Baker et al., Nature. 2006 Aug. 24; 442(7105):916-919). The GRN-related form of FTD is a proteinopathy characterized by the appearance of neuronal inclusions containing ubiquitinated and fragmented TDP-43 (encoded by the TARDBP). In PGRN-deficient mouse models, driving neuronal expression of PGRN using an AAV gene therapy approach has been shown to correct behavioral deficits associated with FTD (Arrant et al. Brain. 2017; 140.5: 1447-1465).

PGRN deficiency is also associated with neuronal ceroid lipofuscinosis 11 (NCL11). In particular, homozygous mutations in GRN are associated with neuronal ceroid lipofuscinosis 11 (NCL11), which is characterized by cerebellar ataxia, seizures, retinitis pigmentosa, and cognitive disorders, usually beginning between 13 and 25 years of age (Faber et al. Brain. 2020; 143(1):303-31).

Thus, there is a strong biological rationale for a therapeutic approach which increases levels of PGRN in the central nervous system to treat neurological diseases associated with PGRN deficiency. The link between PGRN deficiency and CNS disorders, including FTD and NCL11 is discussed in detail in Mole and Cotman, Biochimica et Biophysica Acta. 2015; 1852: 2237-2241, Chitramuthu et al. Brain. 2017; 140: 3081-3104, and Hum et al., Brain. 2020; 143: 303-319).

The nucleotide sequence encoding a PGRN protein used in the nucleic acid constructs of the present invention may encode a human PGRN protein. The nucleotide sequence encoding a PGRN protein used in the nucleic acid constructs of the present invention may encode a wild-type PGRN protein. The nucleotide sequence encoding a PGRN protein used in the nucleic acid constructs of the present invention may encode a wild-type human PGRN protein.

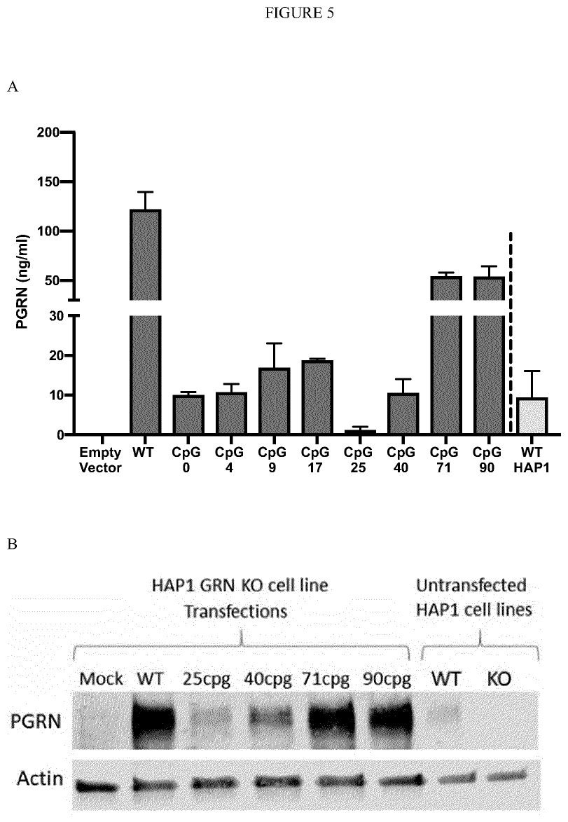

The inventors have found that, for nucleic acid constructs and vectors comprising an MeCP2 promoter, codon optimization of the nucleotide sequence encoding the PGRN protein provided lower PGRN expression levels as compared to a wild type nucleotide sequence encoding PGRN (see Example 5 and FIG. 5). Thus, in some embodiments of the present invention, the nucleotide sequence encoding a PGRN protein is not codon optimized.

A preferred nucleotide sequence encoding a PGRN protein that may be used in the nucleic acid constructs of the present invention comprises or consists of the nucleotide sequence of SEQ ID NO: 12 or a functional variant thereof having at least 70%, 75%, 80%, 85%, 90%, 91%, 92%, 93%, 94%, 95%, 96%, 97%, 98%, 99%, 99.5%, or 99.9% identity to the nucleotide sequence of SEQ ID NO: 12. Fragments of said nucleotide sequences may also be used. Such fragments may be 1000-1781 bp, 1200-1750 bp, 1400-1700 bp, or 1500-1600 bp in length.

A preferred nucleotide sequence encoding a PGRN protein that may be used in the nucleic acid constructions of the present invention encodes a PGRN protein which comprises or consists of the amino acid sequence of SEQ ID NO: 13 or a functional variant thereof having at least 70%, 75%, 80%, 85%, 90%, 91%, 92%, 93%, 94%, 95%, 96%, 97%, 98%, 99%, 99.5%, or 99.9% identity to the amino acid sequence of SEQ ID NO: 13. Nucleotides encoding fragments of said PGRN protein may also be used. Such fragments may be 300-592, 350-490, 400-480, or 450-475 amino acid residues in length.

In any protein or polypeptide described herein, the amino acid sequence may be modified by additions, deletions or substitutions, provided that a polypeptide having the modified sequence exhibits the same activity, as compared to a polypeptide having the unmodified sequence. By “the same” it is to be understood that the polypeptide of the modified sequence does not exhibit significantly reduced activity as compared to a polypeptide of the unmodified sequence. Such a modified protein or the nucleotide sequence which encodes said modified protein may be considered “functional variants”.

The nucleic acid constructs of the present invention may comprise a functional variant or fragment of a nucleotide sequence encoding a PGRN protein described herein. A functional variant or fragment of a nucleotide sequence encoding a PGRN protein described herein may be functional in the sense that it retains the characteristics of the corresponding non-variant or full-length nucleotide sequence encoding a PGRN protein.

The nucleic acid constructs of the present invention may comprise a nucleotide sequence encoding a functional variant or fragment of a PGRN protein described herein. A functional variant or fragment of a PGRN protein described herein may be functional in the sense that it retains the characteristics of the corresponding non-variant or full-length PGRN protein.

Work to characterize the function and intracellular interactions of PRGN remains ongoing. Nevertheless, PGRN has been observed to co-localize with the lysosomal marker protein LAMP-1 (lysosomal-associated membrane protein 1) and to play a role in the regulation of lysosomal function and biogenesis through acidification of lysosomes (Tanaka et al., Human Molecular Genetics. 2017; 26(5): 969-988).

In certain embodiments, the functional variant or fragment of a nucleotide sequence encoding a PGRN protein encodes a PGRN protein which is capable of co-localization with LAMP-1. Co-localization of the PGRN protein encoded by the functional variant or fragment and LAMP-1 may be at least about 50%, 60%, 70%, 75%, 80%, 85%, 90%, 91%, 92%, 93%, 94%, 95%, 96%, 97%, 98%, or 99% of the co-localization between a PGRN protein encoded by the corresponding non-variant or full-length nucleotide sequence and LAMP-1 under the same conditions. Co-localization of the PGRN protein encoded by the functional variant or fragment and LAMP-1 may be substantially the same as, or greater than, the co-localization between a PGRN protein encoded by the corresponding non-variant or full-length nucleotide sequence and LAMP-1 under the same conditions.

In certain embodiments, the functional variant or fragment of a PGRN protein is capable of co-localization with LAMP-1. Co-localization of the variant of fragment of a PGRN protein and LAMP-1 may be at least about 50%, 60%, 70%, 75%, 80%, 85%, 90%, 91%, 92%, 93%, 94%, 95%, 96%, 97%, 98%, or 99% of the co-localization between the corresponding non-variant or full-length PGRN protein under the same conditions. Co-localization of the variant of fragment of a PGRN protein may be substantially the same as, or greater than, the co-localization between the corresponding non-variant or full-length PGRN protein under the same conditions.

Co-localization of a PGRN protein and LAMP-1 may be evaluated and/or quantified using any suitable technique known in the art. For example, cultured cells deficient in PGRN (e.g. GRN^{-/-} cells, or cells in which expression PGRN expression has been down regulated by siRNA) may be transfected with a vector which comprises the nucleic acid construct comprising the functional variant or fragment of the nucleotide sequence encoding the PGRN protein. The cells may then be immunostained using a first fluorescently labelled (e.g. green) antibody specific for PGRN and a second fluorescently labelled (e.g. red) antibody specific for LAMP-1. Co-localization of the red and green staining can then be assessed using a fluorescence microscopy (see Tanaka et al., Human Molecular Genetics. 2017; 26(5): 969-988).

In certain embodiments, the functional variant or fragment of a nucleotide sequence encoding a PGRN protein encodes a PGRN protein which is capable of regulating lysosomal acidification. The regulation of lysosomal acidification by the PGRN protein encoded by the functional variant or fragment may be at least about 50%, 60%, 70%, 75%, 80%, 85%, 90%, 91%, 92%, 93%, 94%, 95%, 96%, 97%, 98%, or 99% of the regulation of lysosomal acidification by a PGRN protein encoded by the corresponding non-variant or full-length nucleotide sequence under the same conditions. The regulation of lysosomal acidification by the PGRN protein encoded by the functional variant or fragment may be substantially the same as, or greater than, the regulation of lysosomal acidification by a PGRN protein encoded by the corresponding non-variant or full-length nucleotide sequence under the same conditions.

In certain embodiments, the functional variant or fragment of a PGRN protein is capable of regulating lysosomal acidification. The regulation of lysosomal acidification by the variant or fragment of a PGRN protein may be at least about 50%, 60%, 70%, 75%, 80%, 85%, 90%, 91%, 92%, 93%, 94%, 95%, 96%, 97%, 98%, or 99% of the regulation of lysosomal acidification by the corresponding non-variant or full-length PGRN protein under the same conditions. The regulation of lysosomal acidification by the variant or fragment of a PGRN protein may be substantially the same as, or greater than, the regulation of lysosomal acidification by the corresponding non-variant or full-length PGRN protein under the same conditions.

In certain embodiments, the functional variant or fragment of a nucleotide sequence encoding a PGRN protein encodes a PGRN protein which is capable of increasing lysosomal acidification. The PGRN protein encoded by the functional variant or fragment may be capable of increasing lysosomal acidification to at least about 50%, 600%, 700%, 75%, 80%, 85%, 900%, 910%, 920%, 930%, 940%, 950%, 960%, 970%, 98%, or 99% of the increase in lysosomal acidification provided by a PGRN protein encoded by the corresponding non-variant or full-length nucleotide sequence under the same conditions. The PGRN protein encoded by the functional variant or fragment may be capable of increasing lysosomal acidification to an extent which is substantially the same as, or greater than, the increase in lysosomal acidification provided by a PGRN protein encoded by the corresponding non-variant or full-length nucleotide sequence under the same conditions.

In certain embodiments, the functional variant or fragment of a PGRN protein is capable of increasing lysosomal acidification. The variant or fragment of a PGRN protein may be capable of increasing lysosomal acidification to at least about 50%, 60%, 70%, 75%, 800%, 850%, 900%, 91%, 92%, 93%, 94%, 95%, 96%, 97%, 98%, or 99% of the increase in lysosomal acidification provided by the corresponding non-variant or full-length PGRN protein under the same conditions. The variant or fragment of a PGRN protein may be capable of increasing lysosomal acidification to an extent which is substantially the same as, or greater than, the regulation of lysosomal acidification by the corresponding non-variant or full-length PGRN protein under the same conditions.

The effect PGRN on lysosomal acidification may be evaluated using any suitable technique in the art. For example, cultured cells deficient in PGRN (e.g. GRN^{-/-} cells, or cells in which expression PGRN expression has been down regulated by siRNA) may be transfected with a vector which comprises the nucleic acid construct comprising the functional variant or fragment of the nucleotide sequence encoding the PGRN protein. The acidification of lysosomes in the transfected cells can then be assessed using cell permeable dyes, such as LysoSensor DND-189 or acridine orange (see Tanaka et al., Human Molecular Genetics. 2017; 26(5): 969-988). LysoSensor DND-189 fluorescence increases dependently on lysosomal acidity. Acridine orange monomer emits green fluorescence, whereas its dimer and oligomers formed when it is protonated. Thus, the ratio of red/green fluorescence indicates the relative acidity of the lysosomes. Fluorescent signal generated by the dyes may be measured using fluorescent microscopy or a fluorescent plate reader.

Any comparison of activity between sequences is to be conducted using the same assay. Unless otherwise specified, modifications to a polypeptide sequence are preferably conservative amino acid substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table A1 below. Where amino acids have similar polarity, this can be determined by reference to the hydropathy scale for amino acid side chains in Table A2.

TABLE A1
Chemical properties of amino acids

| | | | |
|---|---|---|---|
| Ala (A) | aliphatic, hydrophobic, neutral | Met (M) | hydrophobic, neutral |
| Cys I | polar, hydrophobic, neutral | Asn (N) | polar, hydrophilic, neutral |
| Asp (D) | polar, hydrophilic, charged (−) | Pro (P) | hydrophobic, neutral |
| Glu I | polar, hydrophilic, charged (−) | Gln (Q) | polar, hydrophilic, neutral |
| Phe (F) | aromatic, hydrophobic, neutral | Arg I | polar, hydrophilic, charged (+) |
| Gly (G) | aliphatic, neutral | Ser (S) | polar, hydrophilic, neutral |
| His (H) | aromatic, polar, hydrophilic, charged (+) | Thr (T) | polar, hydrophilic, neutral |
| Ile (I) | aliphatic, hydrophobic, neutral | Val (V) | aliphatic, hydrophobic, neutral |
| Lys (K) | polar, hydrophilic, charged(+) | Trp (W) | aromatic, hydrophobic, neutral |
| Leu (L) | aliphatic, hydrophobic, neutral | Tyr (Y) | aromatic, polar, hydrophobic |

TABLE A2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

Vectors

The present invention provides vectors comprising the nucleic acid constructs of the present invention. The vector may be of any type. For example, the vector may be a plasmid vector or a minicircle DNA. Typically however, vectors of the invention are viral vectors. The viral vector may be based on the herpes simplex virus, adenovirus or lentivirus. The viral vector may be an adeno-associated virus (AAV) vector or a derivative thereof. The viral vector derivative may be a chimeric, shuffled or capsid modified derivative.

The viral vector may comprise an AAV genome from a naturally derived serotype, isolate or clade of AAV. The AAV serotype determines the tissue specificity of infection (or tropism) of an AAV virus. Preferably, the AAV used in the present invention is capable of transducing cells of the CNS, for example neuronal cells, astrocytes and/or oligodendrocytes. For example, the AAV serotype may be AAV2, AAV5 or AAV8, preferably AAV2.

The efficacy of gene therapy is, in general, dependent upon adequate and efficient delivery of the donated DNA. This process is usually mediated by viral vectors. Adeno associated viruses (AAV), a member of the parvovirus family, are commonly used in gene therapy. Wild-type AAV, containing viral genes, insert their genomic material into chromosome 19 of the host cell (Kotin, et al. PNAS USA 1990. 87:2211-2215). The AAV single-stranded DNA genome comprises two inverted terminal repeats (ITRs) and two open reading frames, containing structural (cap) and packaging (rep) genes (Hermonat et al., J. Virol 1984. 51:329-339). For therapeutic purposes, the only sequences required in cis, in addition to the therapeutic gene, are the ITRs. The AAV virus is therefore modified: the viral genes are removed from the genome, producing recombinant AAV (rAAV). This contains only the therapeutic gene, the two ITRs. The removal of the viral genes renders rAAV incapable of actively inserting its genome into the host cell DNA. Instead, the rAAV genomes fuse via the ITRs, forming circular, episomal structures, or insert into pre-existing chromosomal breaks. For viral production, the structural and packaging genes, now removed from the rAAV, are supplied in trans, in the form of a helper plasmid. AAV vectors are limited by a relatively small packaging capacity of roughly 4.8 kb.

Most gene therapy vector constructs are based on the AAV serotype 2 (AAV2). AAV2 binds to the target cells via the heparin sulphate proteoglycan receptor (Summerford and Samulski J. Virol, 1998, 72:1438-1445). The AAV2 genome, like those of all AAV serotypes, can be enclosed in a number of different capsid proteins. AAV2 can be packaged in its natural AAV2 capsid (AAV2/2) or it can be pseudotyped with other capsids (e.g. AAV2 genome in AAV1 capsid; AAV2/1, AAV2 genome in AAV5 capsid; AAV2/5 and AAV2 genome in AAV8 capsid; AAV2/8).

The vector of the present invention may comprise an adeno-associated virus (AAV) genome or a derivative thereof.

An AAV genome is a polynucleotide sequence which encodes functions needed for production of an AAV viral particle. These functions include those operating in the replication and packaging cycle for AAV in a host cell, including encapsidation of the AAV genome into an AAV viral particle. Naturally occurring AAV viruses are replication-deficient and rely on the provision of helper functions in trans for completion of a replication and packaging cycle. Accordingly and with the additional removal of the AAV rep and cap genes, the AAV genome of the vector of the invention is replication-deficient.

The AAV genome may be in single-stranded form, either positive or negative-sense, or alternatively in double-stranded form. The use of a double-stranded form allows bypass of the DNA replication step in the target cell and so can accelerate transgene expression. The AAV genome may be from any naturally derived serotype or isolate or clade of AAV. As is known to the skilled person, AAV viruses occurring in nature may be classified according to various biological systems.

Commonly, AAV viruses are referred to in terms of their serotype. A serotype corresponds to a variant subspecies of AAV which owing to its profile of expression of capsid surface antigens has a distinctive reactivity which can be used to distinguish it from other variant subspecies. Typically, a virus having a particular AAV serotype does not efficiently cross-react with neutralizing antibodies specific for any other AAV serotype. AAV serotypes include AAV1, AAV2, AAV3, AAV4, AAV5, AAV6, AAV7, AAV8, AAV9, AAV10 (AAVrH10) and AAV11, also recombinant serotypes, such as Rec2 and Rec3 identified from primate brain. In the vectors of the invention, the genome may be derived from any AAV serotype. The capsid may also be derived from any AAV serotype. The genome and the capsid may be derived from the same serotype or different serotypes. In vectors of the invention, it is preferred that the genome is derived from AAV serotype 2 (AAV2), AAV serotype 4 (AAV4), AAV serotype 5 (AAV5) or AAV serotype 8 (AAV8). It is more preferred that the genome is derived from AAV2.

It is even more preferred that the AAV is AAV-TT. AAV-TT is described in detail in Tordo et al., Brain. 2018; 141(7): 2014-2031 and WO 2015/121501, which are incorporated herein by reference in their entirety.

Reviews of AAV serotypes may be found in Choi et al (Curr Gene Ther. 2005; 5(3); 299-310) and Wu et al (Molecular Therapy. 2006; 14(3), 316-327). The sequences of AAV genomes or of elements of AAV genomes including ITR sequences, rep or cap genes for use in the invention may be derived from the following accession numbers for AAV whole genome sequences: Adeno-associated virus 1 NC_002077, AF063497; Adeno-associated virus 2 NC_001401; Adeno-associated virus 3 NC_001729; Adeno-associated virus 3B NC_001863; Adeno-associated virus 4 NC_001829; Adeno-associated virus 5 Y18065,5AF085716; Adeno-associated virus 6 NC_001862; Avian AAV ATCC VR-865 AY186198, AY629583, NC_004828; Avian AAV strain DA-1 NC_006263, AY629583; Bovine AAV NC_005889, AY388617.

AAV viruses may also be referred to in terms of clades or clones. This refers to the phylogenetic relationship of naturally derived AAV viruses, and typically to a phylogenetic group of AAV viruses which can be traced back to a common ancestor, and includes all descendants thereof. Additionally, AAV viruses may be referred to in terms of a specific isolate, i.e. a genetic isolate of a specific AAV virus found in nature. The term genetic isolate describes a population of AAV viruses which has undergone limited genetic mixing with other naturally occurring AAV viruses, thereby defining a recognizably distinct population at a genetic level. Examples of clades and isolates of AAV that may be used in the invention include:
Clade A: AAV1 NC_002077, AF063497, AAV6 NC_001862, Hu. 48 AY530611, Hu 43 AY530606, Hu 44 AY530607, Hu 46 AY530609;
Clade B: Hu. 19 AY530584, Hu. 20 AY530586, Hu 23 AY530589, Hu22 AY530588, Hu24 AY530590, Hu21 AY530587, Hu27 AY530592, Hu28 AY530593, Hu 29 AY530594, Hu63 AYS30624, Hu64 AY530625, Hul3 AY530578, Hu56 AY530618, Hu57 AY530619, Hu49 AY530612, Hu58 AY530620, Hu34 AY530598, Hu35 AY530599, AAV2 NC_001401, Hu45 AY530608, Hu47 AY530610, Hu51 AY530613, Hu52 AY530614, Hu T41 AY695378, Hu S17 AY695376, Hu T88 AY695375, Hu T71 AY695374, Hu T70 AY695373, Hu T40 AY695372, Hu T32 AY695371, Hu T17 AY695370, Hu LG15 AY695377;
Clade C: Hu9 AY530629, HulO AY530576, Hull AY530577, Hu53 AY530615, Hu55 AY530617, Hu54 AY530616, Hu7 AY530628, Hul8 AY530583, Hul5 AY530580, Hul6 AY530581, Hu25 AY530591, Hu60 AY530622, Ch5 AY243021, Hu3 AY530595, Hul AY530575, Hu4 AY530602 Hu2, AY530585, Hu61 AY530623;
Clade D: Rh62 AY530573, Rh48 AY530561, Rh54 AY530567, Rh55 AY530568, Cy2 AY243020, AAV7 AF513851, Rh35 AY243000, Rh37 AY242998, Rh36 AY242999, Cy6 AY243016, Cy4 AY243018, Cy3 AY243019, Cy5 AY243017, Rhl3 AY243013;
Clade E: Rh38 AY530558, Hu66 AY530626, Hu42 AY530605, Hu67 AY530627, Hu40 AY530603, Hu41 AY530604, Hu37 AY530600, Rh40 AY530559, Rh2 AY243007, Bbl AY243023, Bb2 AY243022, RhlO AY243015, Hul7 AY530582, Hub AY530621, Rh25 AY530557, P12 AY530554, Pil AY530553, Pi3 AY530555, Rh57 AY530569, Rh50 AY530563, Rh49 AY530562, Hu39 AY530601, Rh58 AY530570, Rhbl AY530572, Rh52AY530565, Rh53 AY530566, Rh51 AY530564, Rh64 AY530574, Rh43 AY530560, AAV8 AF513852, Rh8 AY242997, Rhl AY530556; and
Clade F: Hu 14 (AAV9) AY530579, Hu31 AY530596, Hu32 AY530597; Clonal Isolate AAV5 Y18065, AF085716, AAV 3 NC_001729, AAV 3B NC_001863, AAV4 15 NC_001829, Rh34 AY243001, Rh33 AY243002, Rh32 AY243003.

The skilled person can select an appropriate serotype, clade, clone or isolate of AAV for use in the present invention on the basis of their common general knowledge. It should be understood however that the invention also encompasses use of an AAV genome of other serotypes that may not yet have been identified or characterized.

Typically, the AAV genome of a naturally derived serotype or isolate or clade of AAV comprises at least one inverted terminal repeat sequence (ITR). Vectors of the invention may comprise two ITRs, preferably one at each end of the genome. An ITR sequence acts in cis to provide a functional origin of replication, and allows for integration and excision of the vector from the genome of a cell. Preferred ITR sequences are those of AAV2 and variants thereof. The AAV genome typically comprises packaging genes, such as rep and/or cap genes which encode packaging functions for an AAV viral particle. The rep gene encodes one or more of the proteins Rep78, Rep68, Rep52 and Rep40 or variants thereof. The cap gene encodes one or more capsid proteins such as VP1, VP2 and VP3 or variants thereof. These proteins make up the capsid of an AAV viral particle. Capsid variants are discussed below.

Preferably the AAV genome will be derivatised for the purpose of administration to patients. Such derivatisation is standard in the art and the present invention encompasses the use of any known derivative of an AAV genome, and derivatives which could be generated by applying techniques known in the art. Derivatisation of the AAV genome and of the AAV capsid are reviewed in Coura and Nardi (Virology Journal. 2007; 4:99), and in Choi et al, referenced above.

Derivatives of an AAV genome include any truncated or modified forms of an AAV genome which allow for expression of a Rep-1 transgene from a vector of the invention in vivo. Typically, it is possible to truncate the AAV genome significantly to include minimal viral sequence yet retain the above function. This is preferred for safety reasons to reduce the risk of recombination of the vector with wild-type virus, and also to avoid triggering a cellular immune response by the presence of viral gene proteins in the target cell. Typically, a derivative will include at least one inverted terminal repeat sequence (ITR), preferably more than one ITR, such as two ITRs or more. One or more of the ITRs may be derived from AAV genomes having different serotypes, or may be a chimeric or mutant ITR. A preferred mutant ITR is one having a deletion of a trs (terminal resolution site). This deletion allows for continued replication of the genome to generate a single-stranded genome which contains both coding and complementary sequences i.e. a self-complementary AAV genome. This allows for bypass of DNA replication in the target cell, and so enables accelerated transgene expression.

The one or more ITRs will preferably flank the nucleic acid construct of the present invention, i.e. the nucleotide sequence comprising the MeCP2 promoter and the nucleotide sequence encoding the PGRN protein. The inclusion of one or more ITRs is preferred to aid packaging of the vector of the invention into viral particles. In preferred embodiments, ITR elements will be the only sequences retained from the native AAV genome in the derivative. Thus, a derivative will preferably not include the rep and/or cap genes of the native genome and any other sequences of the native genome. This is preferred for the reasons described above, and also to reduce the possibility of integration of the vector into the host cell genome. Additionally, reducing the size of the AAV genome allows for increased flexibility in incorporating other sequence elements (such as regulatory elements) within the vector in addition to the transgene.

With reference to the AAV2 genome, the following portions could therefore be removed in a derivative of the invention: One inverted terminal repeat (ITR) sequence, the replication (rep) and capsid (cap) genes. However, in some embodiments, including in vitro embodiments, derivatives may additionally include one or more rep and/or cap genes or other viral sequences of an AAV genome. A derivative may be a chimeric, shuffled or capsid-modified derivative of one or more naturally occurring AAV viruses. The invention encompasses the use of capsid protein sequences from different serotypes, clades, clones, or isolates of AAV within the same vector. The invention also encompasses the packaging of the genome of one serotype into the capsid of another serotype i.e. pseudotyping. Chimeric, shuffled or capsid-modified derivatives may be selected to provide one or more desired functionalities for the viral vector. Thus, these derivatives may display increased efficiency of gene delivery, decreased immunogenicity (humoral or cellular), an altered tropism range and/or improved targeting of a particular cell type compared to an AAV viral vector comprising a naturally occurring AAV genome, such as that of AAV2. Increased efficiency of gene delivery may be effected by improved receptor or co-receptor binding at the cell surface, improved internalization, improved trafficking within the cell and into the nucleus, improved uncoating of the viral particle and improved conversion of a single-stranded genome to double-stranded form. Increased efficiency may also relate to an altered tropism range or targeting of a specific cell population, such that the vector dose is not diluted by administration to tissues where it is not needed.

Chimeric capsid proteins include those generated by recombination between two or more capsid coding sequences of naturally occurring AAV serotypes. This may be performed for example by a marker rescue approach in which non-infectious capsid sequences of one serotype are co-transfected with capsid sequences of a different serotype, and directed selection is used to select for capsid sequences having desired properties. The capsid sequences of the different serotypes can be altered by homologous recombination within the cell to produce novel chimeric capsid proteins. Chimeric capsid proteins also include those generated by engineering of capsid protein sequences to transfer specific capsid protein domains, surface loops or specific amino acid residues between two or more capsid proteins, for example between two or more capsid proteins of different serotypes. Shuffled or chimeric capsid proteins may also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes may also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N- and/or C-terminus of a capsid coding sequence. The unrelated protein or peptide may advantageously be one which acts as a ligand for a particular cell type, thereby conferring improved binding to a target cell or improving the specificity of targeting of the vector to a particular cell population. The unrelated protein may also be one which assists purification of the viral particle as part of the production process i.e. an epitope or affinity tag. The site of insertion will typically be selected so as not to interfere with other functions of the viral particle e.g. internalisation, trafficking of the viral particle. The skilled person can identify suitable sites for insertion based on their common general knowledge. Particular sites are disclosed in Choi et al, referenced above.

The invention additionally encompasses the use of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

The present invention also provides an AAV viral particle comprising a vector of the invention. The AAV particles of the invention include transcapsidated forms wherein an AAV genome or derivative having an ITR of one serotype is packaged in the capsid of a different serotype. The AAV particles of the invention also include mosaic forms wherein a mixture of unmodified capsid proteins from two or more different serotypes makes up the viral envelope. The AAV particle also includes chemically modified forms bearing ligands adsorbed to the capsid surface. For example, such ligands may include antibodies for targeting a particular cell surface receptor.

The vectors, including AAV vectors, of the invention and AAV viral particles of the invention may be prepared by standard means known in the art for provision of vectors for gene therapy. Thus, well established public domain transfection, packaging and purification methods can be used to prepare a suitable vector preparation.

Nucleic acid constructs and vectors of the invention comprising a nucleotide sequence encoding a PGRN protein have the ability to rescue loss of PGRN function, which may occur for example by mutations in one or both alleles of a GRN gene of a patient. “Rescue” generally means any amelioration or slowing of progression of a phenotype associated with PRGN deficiency, for example restoring the presence of PGRN protein in the brain and/or a reducing neuronal pathologies.

The properties of nucleic acid constructs and vectors of the present invention may be tested using techniques known by the person skilled in the art. For example a nucleic acid construct of the invention can be assembled into a vector of the invention and delivered to a PRGN deficient test animal, such as a mouse or a primate, and the effects observed and compared to a control.

SEQ ID NO: 10 corresponds to the nucleotide sequence of construct pPG36, which comprises the MeCP2_2 promoter. In certain embodiments, the nucleic acid construct or the viral vector of the present invention comprises or consists of the nucleotide sequence of SEQ ID NO: 10 or functional variant or fragment thereof having at least 70%, 75%, 800%, 850%, 900%, 91%, 920%, 930%, 940%, 95%, 960%, 970%, 980%, 990%, 99.5%, or 99.9% identity to the nucleotide sequence of SEQ ID NO: 10.

SEQ ID NO: 11 corresponds to nucleotide sequence of construct pPG35, which comprises the MeCP2_1 promoter. In certain embodiments, the nucleic acid construct or the viral vector of the present invention comprises or consists of the nucleotide sequence of SEQ ID NO: 11 or functional variant or fragment thereof having at least 70%, 750%, 800%, 850%, 900%, 91%, 920%, 930%, 940%, 95%, 960%, 970%, 980%, 990%, 99.5%, or 99.9% identity to the nucleotide sequence of SEQ ID NO: 11.

SEQ ID NO: 17 corresponds to the nucleotide sequence of AAVTT-pPG36, i.e. an AAVTT vector genome comprising the nucleotide sequence of construct pPG36. In certain embodiments, the viral vector of the present invention, such as an AAV vector or an AAVTT vector, comprises or consists of the nucleotide sequence of SEQ ID NO: 17 or functional variant or fragment thereof having at least 70%, 75%, 80%, 85%, 90%, 91%, 92%, 93%, 94%, 95%, 96%, 97%, 98%, 99%, 99.5%, or 99.9% identity to the nucleotide sequence of SEQ ID NO: 17.

SEQ ID NO: 18 and 19 correspond to the nucleotide sequence of two alternative AAVTT vector genomes designated AAVTT-p1PG36 and AAVTT-p2PG36, respectively. Both AAVTT-p1PG36 (SEQ ID NO: 18) and AAVTT-p2PG36 (SEQ ID NO: 19) comprise the nucleotide sequence of SEQ ID NO: 17. In certain embodiments, the viral vector of the present invention, such as an AAV vector or an AAVTT vector, comprises or consists of the nucleotide sequence of SEQ ID NO: 18 or a functional variant or fragment thereof having at least 70%, 75%, 80%, 85%, 90%, 91%, 92%, 93%, 94%, 95%, 96%, 97%, 98%, 99%, 99.5%, or 99.9% identity to the nucleotide sequence of SEQ ID NO: 18. In certain embodiments, the viral vector of the present invention, such as an AAV vector or an AAVTT vector, comprises or consists of the nucleotide sequence of SEQ ID NO: 19 or a functional variant or fragment thereof having at least 70%, 75%, 80%, 85%, 90%, 91%, 92%, 93%, 94%, 95%, 96%, 97%, 98%, 99%, 99.5%, or 99.9% identity to the nucleotide sequence of SEQ ID NO: 19.

Pharmaceutical Compositions and Dosages

The nucleic acid constructs and vectors of the present invention can be formulated into pharmaceutical compositions. The present invention thus provides a pharmaceutical composition comprising a nucleic acid construct of the invention, a vector of the invention and/or a viral vector of the invention together with a pharmaceutically acceptable carrier, excipient or diluent.

The pharmaceutical composition of the invention may comprise a pharmaceutically acceptable excipient, carrier, buffer, stabilizer or other materials well known to those skilled in the art. Such materials should be non-toxic and should not interfere with the efficacy of the active ingredient. The precise nature of the carrier or other material may be determined by the skilled person according to the route of administration.

The pharmaceutical composition may be provided in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution or glycols such as ethylene glycol, propylene glycol or polyethylene glycol may be included. In some cases, a surfactant, such as pluronic acid (PF68) 0.001% may be used.

For injection at the site of affliction, the active ingredient will be in the form of an aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection, Hartmann's solution. Preservatives, stabilizers, buffers, antioxidants and/or other additives may be included, as required. For delayed release, the vector may be included in a pharmaceutical composition which is formulated for slow release, such as in microcapsules formed from biocompatible polymers or in liposomal carrier systems according to methods known in the art.

Dosages and dosage regimes can be determined within the normal skill of the medical practitioner responsible for administration of the composition.

Methods of Therapy and Medical Use

The present invention also encompasses the use of the nucleic acid constructs, vectors, viral vectors and pharmaceutical compositions described herein in treating or preventing a disease or condition in a patient.

The present invention thus provides a nucleic acid construct of the invention, a vector of the invention, a viral vector of the invention, and/or a pharmaceutical composition of the invention for use in a method of treating or preventing a disease or a condition in a patient in need thereof. The present invention further provides a method of treating or preventing a disease or condition in a patient in need thereof, said method comprising administering to the patient a therapeutically effective amount of a nucleic acid construct of the invention, a vector of the invention, a viral vector of the invention, and/or a pharmaceutical composition of the invention. The present invention also provides the use of a nucleic acid construct of the invention, a vector of the invention, a viral vector of the invention, and/or a pharmaceutical composition of the invention for the manufacture of a medicament for the treatment or prevention of a disease or condition in a patient in need thereof.

The disease or condition may be characterized by PGRN deficiency. Said PGRN deficiency may arise as a result of a loss of function mutation in one or both alleles of a GRN gene of the patient to be treated.

Thus, the present invention thus provides a nucleic acid construct of the invention, a vector of the invention, a viral vector of the invention, and/or a pharmaceutical composition of the invention for use in a method of treating or preventing a disease characterized by progranulin (PGRN) deficiency in a patient in need thereof. The present invention further provides a method of treating or preventing a disease characterized by progranulin (PGRN) deficiency in a patient in need thereof, said method comprising administering to the patient a therapeutically effective amount of a nucleic acid construct of the invention, a vector of the invention, a viral vector of the invention, and/or a pharmaceutical composition of the invention. The present invention also provides the use of a nucleic acid construct of the invention, a vector of the invention, a viral vector of the invention, and/or a pharmaceutical composition of the invention for the manufacture of a medicament for the treatment or prevention of a disease characterized by progranulin (PGRN) deficiency.

The disease characterized by PGRN deficiency to be treated with a nucleic acid construct of the invention, a vector of the invention, a viral vector of the invention, and/or a pharmaceutical composition of the invention may be a disease of the central nervous system (CNS).

The disease characterized by PGRN deficiency to be treated with a nucleic acid construct of the invention, a vector of the invention, a viral vector of the invention, and/or a pharmaceutical composition of the invention may be frontotemporal dementia (FTD).

The disease characterized by PGRN deficiency to be treated with a nucleic acid construct of the invention, a vector of the invention, a viral vector of the invention, and/or a pharmaceutical composition of the invention may be neuronal ceroid lipofuscinosis type 11 (NCL11).

The disease characterized by PGRN deficiency to be treated with a nucleic acid construct of the invention, a vector of the invention, a viral vector of the invention, and/or a pharmaceutical composition of the invention may be further characterized by lysosomal dysfunction, such as a dysregulation of lysosomal acidification. Said lysosomal dysfunction may be characterized by increased expression levels and/or activity of cathepsin D, preferably mature heavy and/or light chain cathepsin D.

The patient in need of treatment with a nucleic acid construct of the invention, a vector of the invention, a viral vector of the invention, and/or a pharmaceutical composition of the invention may be male or female. Said patient may have been previously identified as being at risk of, or having, a disease characterized by PGRN deficiency. Said patient may have been previously identified as being at risk of, or having, FTD. Said patient may have been previously identified as being at risk of, or having, NCL11.

The dose of a vector of the invention may be determined according to various parameters, especially according to the age, weight and condition of the patient to be treated; the route of administration; and the required regimen. A physician will be able to determine the required route of administration and dosage for any particular patient.

The nucleic acid constructs, vectors, viral vectors, or pharmaceutical compositions of the invention may be administered to the brain and/or the cerebrospinal fluid (CSF) of the patient. The delivery to the brain may be selected from intracerebral delivery, intraparenchymal delivery, intraputaminal delivery, and combinations thereof. Further target regions in the brain may include the thalamus, cerebellum, subthalamic nucleus, and combinations thereof. The delivery to the CSF may be selected from intra-cisterna magna delivery, intrathecal delivery, intracerebroventricular (ICV) delivery, and combinations thereof.

The delivery to the brain and/or the cerebrospinal fluid (CSF) of the patient may be by injection. The injection to the brain may be selected from intracerebral injection, intraparenchymal injection, intraputaminal injection, and combinations thereof. The delivery to the CSF may be selected from intra-cisterna magna injection, intrathecal injection, intracerebroventricular (ICV) injection, and combinations thereof.

The injection to the brain and/or the cerebrospinal fluid may comprise convection enhanced delivery (CED). The CED procedure involves a minimally invasive surgical exposure of the brain, followed by placement of small diameter catheters directly into the target area of the brain. CED is described, for example by Debinski et al. (2009) Expert Rev Neurother. 9(10):1519-27.

The dose of the nucleic acid construct, vector, viral vector or pharmaceutical composition of the invention may be provided as a single dose, but may be repeated in cases where vector may not have targeted the correct region. The treatment is preferably a single injection, but repeat injections, for example in future years and/or with different AAV serotypes may be considered.

Host Cells

The present invention additionally provides a host cell comprising a nucleic acid construct of the invention, a vector of the invention, a viral vector of the invention and/or an AAV viral particle of the invention. The present invention also provides a host cell which produces a viral vector of the invention and/or an AAV particle of the invention.

Any suitable host cell may comprise a nucleic acid construct of the invention, a vector of the invention, a viral vector of the invention and/or an AAV viral particle of the invention. Further, any suitable host cell can be used to produce a viral vector of the invention and/or an AAV particle of the invention. In general, such cells will be transfected mammalian cells but other cell types, e.g. insect cells, can also be used. In terms of mammalian cell production systems, HEK293 and HEK293T are preferred for AAV vectors. BHK or CHO cells may also be used.

Kits

The present invention further provides kits comprising the nucleic acid constructs of the invention, the vectors of the invention, the viral vectors of the invention, and/or pharmaceutical compositions of the present invention.

The present invention is further illustrated by the following examples that, however, are not to be construed as limiting the scope of protection. The features disclosed in the foregoing description and in the following examples may, both separately and in any combination thereof, be material for realizing the invention in diverse forms thereof.

### EXAMPLES

### Example 1—Materials and methods

Cell Culture

HEK293T cells were obtained from the American Tissue Collection Center (ATCC, Manassas, VA, USA) and were maintained in Dulbecco's modified Eagle's medium (DMEM) supplemented with 10% FBS and 1% penicillin/streptomycin. A549 cells were obtained from Sigma Aldrich (St Louis, MO, USA) and were maintained in Kaighn's modification of Ham's F-12 medium (F-12K) supplemented with 10% FBS and 1% penicillin/streptomycin. CaSki cells were obtained from the ATCC (Manassas, VA, USA) and were maintained Roswell Park Memorial Institute's 1640 medium (RPMI-1640) supplemented with 10% FBS and 1% penicillin/streptomycin. COS-7 cells were obtained from the ATCC (Manassas, VA, USA) and were maintained in Dulbecco's modified Eagle's medium (DMEM) supplemented with 10% FBS and 1% penicillin/streptomycin. VERO cells were obtained from ATCC (Manassas, VA, USA) and were maintained in Eagle's minimum essential medium (EMEM) supplemented with 10% FBS and 1% penicillin/streptomycin. Neuro-2A cells were obtained from the ATCC (Manassas, VA, USA) and were maintained in Eagle's minimum essential medium (EMEM) supplemented with 10% FBS and 1% penicillin/streptomycin. NIH3T3 cells were obtained from ATCC (Manassas, VA, USA) and were maintained in Dulbecco's modified Eagle's medium (DMEM) supplemented with 10% FBS and 1% penicillin/streptomycin. HAP1 cells and HAP1 GRN KO cells were obtained from Horizon Discovery (Waterbeach, United Kingdom) and were maintained in Iscove's Modified Dulbecco's Medium (IMEM) supplemented with 10% FBS and 1% penicillin/streptomycin.

Codon Optimization

Codon optimized nucleotide sequences encoding PGRN having reduced CpG content were generated. The codon optimized sequences were designated “CpG X” where X denotes the percentage of wild-type CpG sites retained in the codon optimized sequence. For example, the nucleotide sequence designated CpG 90 comprises 90% of the CpG sites of the corresponding wild-type sequence. The resulting sequences were cloned into an expression vector.

Lentivirus Production

All lentiviral vectors used were second generation and were produced using standard viral production methods previously described in Salmon, P. and D. Trono (‘Production and titration of lentiviral vectors’. Curr Protoc Neurosci, 2006. Chapter 4: Unit 4.21). Briefly, 5.7 million HEK293T cells were plated per 10 cm dish. The following day, cells were transfected with lipofectamine2000 (ThermoFisher) with 10 μg of transfer vector, 3 μg of pMD2G and 8 μg psPAX2. The media was changed 12-14 hrs post-transfection. The viral supernatant was collected 24 and 48 hrs after this media change for a total of 20 mL of virus, and passed through a 0.45 μm filter. The viral supernatant was concentrated to 20× in PBS using Lenti-XTM concentrator (CloneTech) prior to being snap frozen.

Lentivirus Titration

All lentiviruses were titered using Lenti-X qRT-PCR Titration Kit (Takara).

Lentiviral Transduction

Cells were resuspended and plated into equal concentration of viral supernatant supplemented with 4 ug/ml polybrene. The viral supernatant was exchanged for fresh medium 12-24 h later. Cos-8, NIH3T3, A549, CaSKi, HEK293T, SK-N-SH cells were transduced at a MOI of 200. VERO and Neuro-2A cells were transduced at a MOI of 1000.

Western Blot Analysis

HEK293T cells were transfected with constructs of interest using lipofectamine 2000 (ThermoFisher) per the manufacturer's instructions. Two days post transfection, cells were lysed in RIPA buffer (Sigma-Aldrich) supplemented with protease inhibitor cocktail (Sigma-Aldrich). Protein concentration was measured using BCA protein assay reagent (ThermoFisher) and Varioskab LUX Microplate Reader (ThermoFisher). Lysates were mixed with loading buffer; equal amounts of protein were run on Mini-PROTEAN TGX 4-15% precast polyacrylamide gels (Bio-Rad) and transferred to nitrocellulose membranes using the Trans-Blot Turbo System (Bio-Rad). Nonspecific antibody binding was blocked with Intercept TBS blocking buffer (Li-Cor) for 1 hr at room temperature. The membranes were incubated with the following primary antibodies: anti-PGRN (1:200 dilution, AF2420, R&D Systems) in Intercept T20 TBS (Li-Cor) overnight at 4° C.; anti-Actin (1:5000 dilution, Sigma-Aldrich, A2066) in Intercept T20 TBS (Li-Cor) overnight at 4° C. The membranes were washed with TBST for 15 min and incubated for 45 min with Donkey anti-goat 680 RD (Li-Cor, 1:5000) and Donkey anti-rabbit 800 CW (Li-Cor, 1:5000) antibodies in Intercept T20 TBS and subsequently washed with TBST for 15 min. Membranes were visualized using the Odyssey CLx (Li-Cor).

GRN^{+/+} HAP-1 (wild-type) cells and GRN^{-/-} HAP-1 (KO) cells were transfected with constructs of interest using lipofectamine 2000 (ThermoFisher) per the manufacturer's instructions. Two days post transfection, cells were lysed in RIPA buffer (Sigma-Aldrich). Protein concentration was measured using Pierce BCA protein assay reagent (ThermoFisher) and SpectraMax i3×Multiplate Reader (Molecular Devices). Lysates were mixed with loading buffer; equal amounts of protein were run on Mini-PROTEAN TGX 4-15% precast polyacrylamide gels (Bio-Rad) and transferred to nitrocellulose membranes using the Trans-Blot Turbo System (Bio-Rad). Nonspecific antibody binding was blocked with Intercept TBS blocking buffer (Li-Cor) for 1 hr at room temperature. The membranes were incubated with the following primary antibodies: anti-PGRN (1:200 dilution, AF2420, R&D Systems) in Intercept T20 TBS (Li-Cor) overnight at 4° C.; anti-Actin (1:10000 dilution, Sigma-Aldrich, A2066) in Intercept T20 TBS (Li-Cor) overnight at 4° C. The membranes were washed with TBST for 15 min and incubated for 1 hr with donkey anti-goat 680 RD (Li-Cor, 1:5000) and donkey anti-mouse 800 CW (Li-Cor, 1:5000) antibodies in Intercept T20 TBS and subsequently washed with TBST for 15 min. Membranes were visualized using the Odyssey CLx (Li-Cor).

Neuron-Astrocyte Co-Culture and Transduction

Primary neuron-astrocyte co-cultures were prepared from embryonic day 17, C57BL/6J mice (Janvier Labs, France). Freshly dissected cortical tissue was first dissociated using papain solution (Sigma Aldrich, P4762). Cells were diluted in neuronal attachment medium and plated (10000 cells/well) on 96-well plates pre-coated with poly-D-Lysine (CORNING 356692). The neuronal attachment media consists of Neurobasal plus medium (ThermoFisher Scientific, A3582901) supplemented with 2.5% heat inactivated fetal bovine serum (ThermoFisher Scientific, A3840002), 1 mM sodium pyruvate (ThermoFisher Scientific, 11360070), 2 mM Glutamax-100×(ThermoFisher Scientific, 35050061), B27 Plus Supplement (ThermoFisher Scientific, 17504044), and 50 units/ml penicillin/streptomycin (ThermoFisher Scientific, 15070063). Cells were maintained by supplementing with fresh serum-free neurobasal medium every week. Lentiviral mediated transduction was performed on DIV 3 (Days In Vitro). Lentivirus stocks were diluted in culture medium and applied on top of cells at given MOI (Multiplicity of Infection) as indicated in figure legends. At DIV 14, i.e. 10-days post transduction, cells were fixed, and immunocytochemistry performed.

Immunolabeling and Imaging

Immunocytochemistry was performed following transduction in primary neurons and astrocytes. Cells were washed three times (1× PBS) followed by fixation using 4% PFA (ThermoFischer Scientific, 28908) for 10 min at room temperature. Cells were then permeabilized using 0.25%-Triton-X/3%-BSA/1×-PBS solution for 10 min (BSA: Bovine Serum Albumin, VWR, 1005-30-1L; Triton-X-100 from Sigma Aldrich, T8787). Following permeabilization, cells were blocked using 3%-BSA/1×-PBS solution for 30 min. Cells were then labeled with primary antibodies (60 min) followed by fluorescent conjugated secondary antibodies (45 min) (see below for the list of antibodies). Imaging was performed on Zeiss LSM 880 (SH-SY5Y cells) and Perkin Elmer Opera Phenix (Neurons/Astrocytes) instruments. Thresholding and Quantification was performed on Image J or Perkin Elmer Harmony software.

| | | | | |
|---|---|---|---|---|
| | Host | | | |
| Name | Species | Catalog No. | Supplier | Dilution |

PRIMARY ANTIBODIES

| | | | | |
|---|---|---|---|---|
| NeuN | Mouse | MAB377 | Millipore | 1:2500 |
| GFAP | Rabbit | Z0334 | DAKO | 1:2500 |
| Human Progranulin | Goat | AF2420 | R&D Systems | 1:1000 |

SECONDARY ANTIBODIES

| | | | | |
|---|---|---|---|---|
| Anti-Goat 550 | Donkey | A32816 | ThermoFisher | 1:600 |
| Anti-Mouse 488 | Donkey | A32766 | ThermoFisher | 1:600 |
| Anti-Rabbit 647 | Donkey | A32795 | ThermoFisher | 1:600 |

ELISA

A human progranulin ELISA kit (AG-45A-0018YEK-KI01, Adipogen) was used to quantify the level of secreted human PGRN following transduction of mice neuron-astrocyte co-culture. The cell culture media was collected 10-days post transduction. Samples were diluted to 1:100 and ELISA measurement performed as per the supplier's instructions. The colorimetric reaction was measured using standard plate reader (Flex Station3, Molecular Devices).

A human progranulin ELISA kit (DPGRN0, RD Systems) was used to quantify the level of secreted human progranulin following transfection GRN^{+/+} HAP-1 (wild-type) cells and GRN^{-/-} HAP-1 (KO) cells. The cell culture media was collected 24 hours post transfection. ELISA measurement performed as per the suppliers' instruction. The colorimetric reaction was measured on a SpectraMax i3×Multiplate Reader (Molecular Devices).

Brain Sectioning, Immunohistochemistry and Acquisitions

Brain sectioning was performed at Neuroscience Associates (TN, USA). First, brains were treated overnight with 20% glycerol and 2% dimethyl sulfoxide to prevent freeze-artifacts, and embedded in a gelatin matrix using MultiBrain® Technology. After curing, the blocks were rapidly frozen by immersion in isopentane chilled to −70° C. with crushed dry ice, and mounted on the freezing stage of an A0860 sliding microtome. The MultiBrain® blocks were sectioned in the coronal plane at 40 μm. All sections were collected sequentially into 24 containers per block that were filled with Antigen Preserve solution (49% PBS pH 7.0, 50% Ethylene glycol, 1% Polyvinyl Pyrrolidone). Sections not stained immediately were stored at −20° C.

Free-floating sections were stained by immunochemistry with an antibody to human progranulin (R&D—AF2420), diluted at 1:15.000. All incubation solutions from the blocking serum onward used Tris buffered saline (TBS) with Triton X-100 as the vehicle; all rinses were with TBS. Endogenous peroxidase activity was blocked by 0.9% hydrogen peroxide treatment and non-specific binding was blocked with 1.26% whole normal serum. Following rinses, the sections were stained with a primary antibody overnight at room temperature. Vehicle solution contained 0.3% Triton X-100 for permeabilization. Following rinses, sections were incubated with an avidin-biotin-HRP complex (Vectastain Elite ABC kit, Vector Laboratories, Burlingame, CA) for one hour at room temperature. Following rinses, the sections were treated with diaminobenzidine tetrahydrochloride (DAB) and 0.0015% hydrogen peroxide to create a visible reaction product, mounted on gelatinized (subbed) glass slides, air-dried, lightly stained with thionine, dehydrated in alcohols, cleared in xylene, and cover-slipped with Permount mounting media. Digital images of stained sections were obtained using an AxioScan Z1 slide scanner with a 20×objective (Zeiss).

AAV Vectors

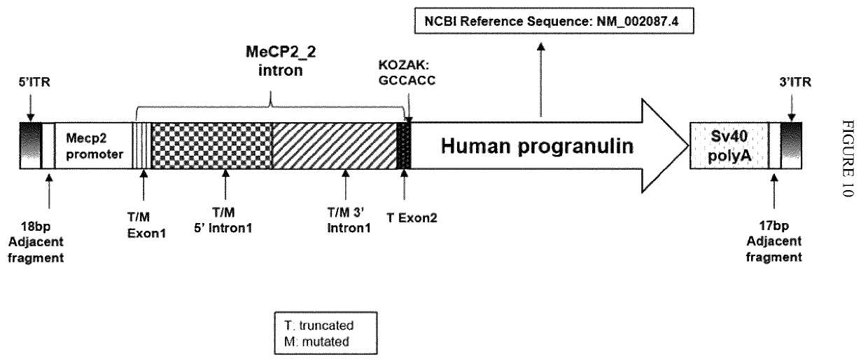

AAV vectors were sourced from two different providers. The corresponding plasmid sequences are provided in SEQ ID NO: 18 (AAVTT-p1PG36) and SEQ ID NO: 19 (AAVTT-p2PG36). AAV vectors were produced using a triple plasmid transfection method as described previously in Grieger et al (‘Production of Recombinant Adeno-associated Virus Vectors Using Suspension HEK293 Cells and Continuous Harvest of Vector From the Culture Media for GMP FIX and FLT1 Clinical Vector’ Molecular Therapy vol. 24 no. 2, 287-297feb. 2016) using HEK 293T or HEK293 cells respectively, including a helper plasmid, a Rep/Cap encoding plasmid, and a plasmid comprising SEQ ID NO: 17 (AAVTT-pPG36). FIG. 10 provides a schematic showing the component parts of the nucleotide sequence of SEQ ID NO: 17.

### Example 2—Generation and Evaluation of Engineered Promoter Constructs

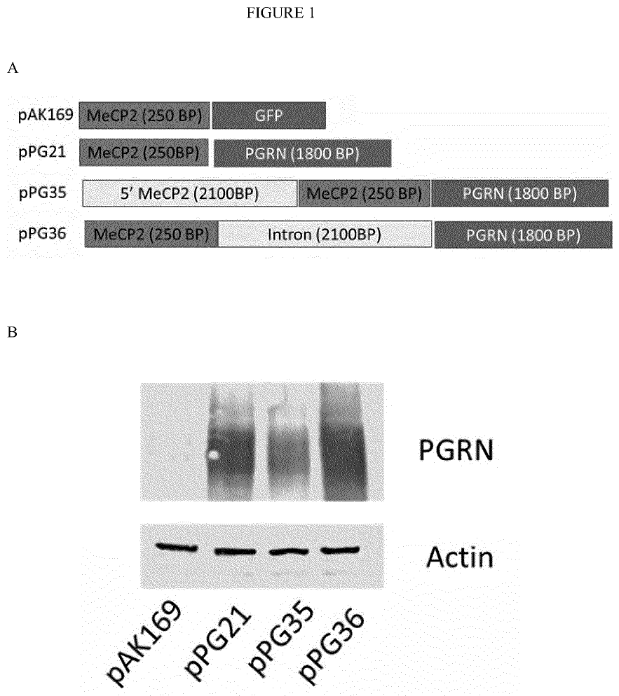

Lentiviral vector constructs pAK169, pPG21, pPG35 and pPG36 were generated as described above. All of these constructs comprise a neuronal specific MeCP2 promoter sequence, as shown in FIG. 1A. Constructs pAK169 and pPG21 each comprise a minimal MeCP2 promoter sequence of 229 bp in length (SEQ ID NO: 1).

Construct pPG35 (SEQ ID NO: 10) comprises an engineered promoter region designated MeCP2_1 (SEQ ID NO: 8). The MeCP2_1 promoter comprises the minimal promoter sequence (SEQ ID NO: 1) and a natural intron. The natural intron is a 2108 bp nucleotide sequence of the murine MeCP2 gene of bp in length (SEQ ID NO: 9) and is arranged 5′ to the minimal promoter sequence. The MeCP2_1 promoter sequence is 2337 bp in length.

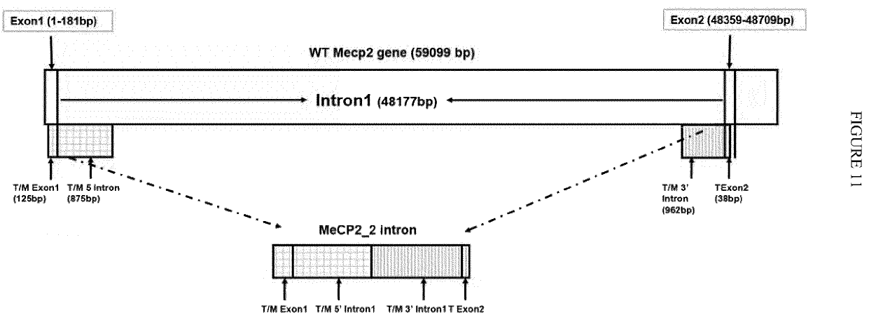

Construct pPG36 (SEQ ID NO: 11) comprises an engineered promoter region designated MeCP2_2 (SEQ ID NO: 3). The MeCP2_2 promoter comprises the minimal MeCP2 promoter sequence (SEQ ID NO: 1) and a 2006 bp synthetic intron (SEQ ID NO: 2), which is arranged 3′ to the minimal promoter sequence. The MeCP2_2 promoter is 2235 bp in length. The synthetic intron (MeCP2_2 intron; SEQ ID NO: 2) was constructed from two intronic sequences and two silenced (i.e. non-expressing) exons of the murine MECP2 gene. The exons were silenced by directed mutation to remove start codons. See FIG. 11 for a schematic showing the construction of the MeCP2_2 intron (SEQ ID NO: 2).

5′ to 3′ the engineered MeCP2_2 promoter comprises: the MeCP2 minimal promoter sequence (SEQ ID NO: 1), Age1 restriction site (ACCGGT; SEQ ID NO 14), Exon1 (SEQ ID NO: 5), 5′ intron (SEQ ID NO: 6), 3′ intron (SEQ ID NO: 7), and Exon2 (SEQ ID NO: 8).

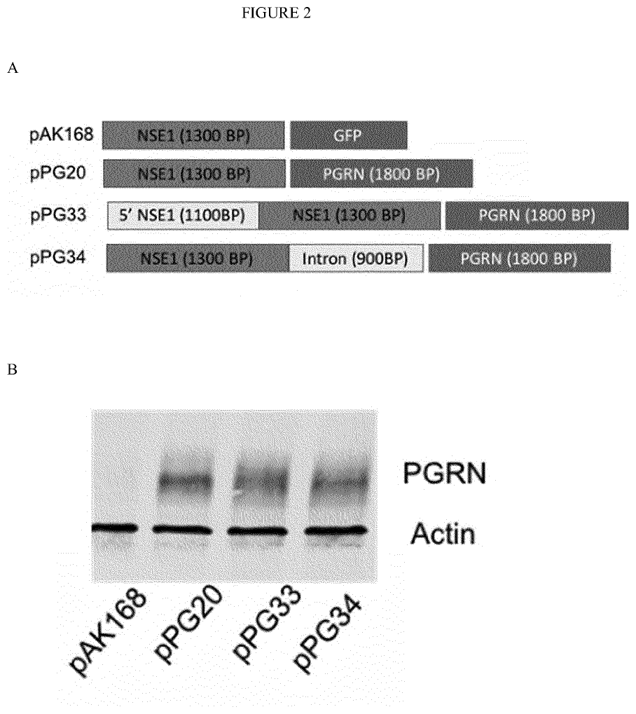

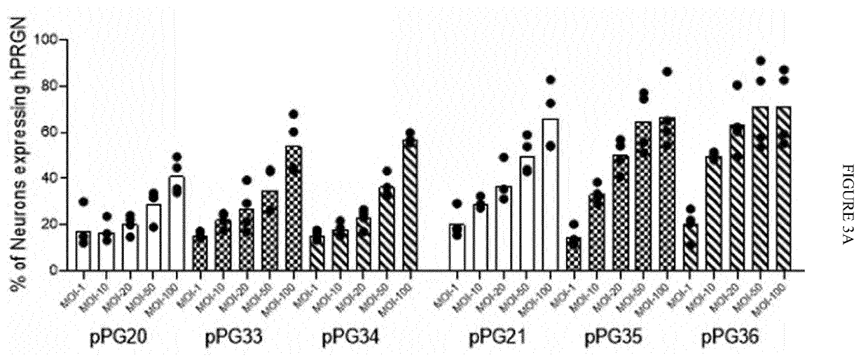

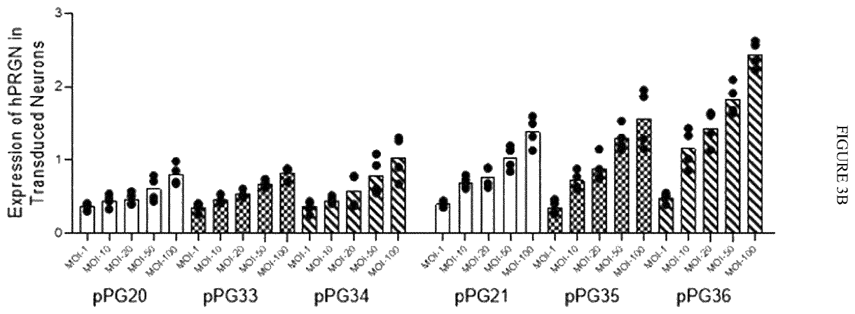

Control lentiviral vector constructs pAK168, pPG20, pPG33 and pPG34 were also generated. Each of these constructs comprises a neuronal specific NSE1 promoter sequence, as shown in FIG. 2A. Constructs pAK168 and pPG20 comprise a minimal NSE1 promoter sequence of about 1300 bp. Constructs pAK168 and pPG20 were used as the equivalent controls for pAK169 and pPG21, respectively.

Construct pPG33 comprises an engineered promoter region designated NSE1_1, which the minimal promoter sequence and a 1100 bp naturally occurring sequence of the human NSE1 gene, which is arranged 5′ to the minimal promoter sequence. Construct pPG34 comprises an engineered promoter region designated NSE_2, which comprises an synthetic intron of about 0.9 kb in length. Constructs pPG33 and pPG34 were used as the equivalent controls for pPG35 and pPG36, respectively.

HEK293T cells were transfected with each of the MeCP2 and NSE1 vectors. Expression of PGRN was evaluated by western blot. The results of these experiments are shown in FIGS. 1B and 2B.

The constructs comprising MeCP2 promoters, i.e. pPG21, pPG35 and pPG36, provided higher PGRN expression levels than those constructs comprising NSE1 promoters (i.e. pPG20, pPG33 and pPG34).

It was also observed that construct pPG36 (comprising the MeCP2_2 promoter) provided higher PGRN expression levels relative to constructs pPG21 (comprising the minimal MeCP2 promoter sequence) and pPG35 (comprising the MeCP2_1 promoter).

### Example 3—Evaluation of Transgene Expression by NSE1 and MeCP2 Promoters in Primary Neurons and Astrocytes

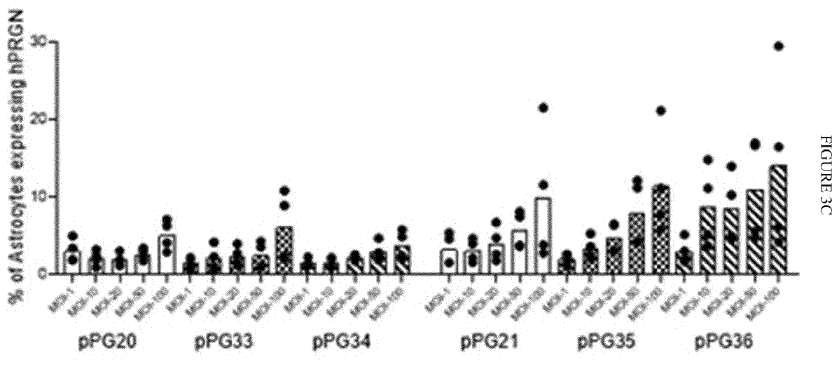

Wild-type murine primary cortical neuron-astrocyte co-cultures were transduced to express human progranulin protein using lentivirus. Lentiviruses were applied at different multiplicity of infection (MOIs) as depicted in FIG. 3. 10-days post lentiviral transduction, cells were fixed and immunolabeled using NeuN (neuron marker), GFAP (astrocyte marker) and human progranulin antibody. The percentage of cells transduced is shown FIGS. 3A (neurons) and 3C (astrocytes) and the expression level (fluorescence intensity/cell) is shown FIGS. 3B (neurons) and 3D (astrocytes).

It was found that constructs comprising engineered NSE1 promoters (pPG33 and pPG34) did not change the transduction efficiency or expression level compared to construct pPG20, which comprises the minimal NSE1 promoter. In contrast, constructs comprising promoters MeCP2_1 and MeCP2_2 (pPG35 and pPG36, respectively) increased the transduction efficiency or expression level compared to construct pPG21, which comprises the minimal MeCP2 promoter. Notably, the MeCP2_2 promoter (pPG36) performed best of all the promoters tested in terms of transduction efficiency and PGRN expression levels.

### Example 4—Evaluation of PGRN Secretion by Neurons and Astrocytes Transfected with Vectors Comprising MeCP2 Promoters

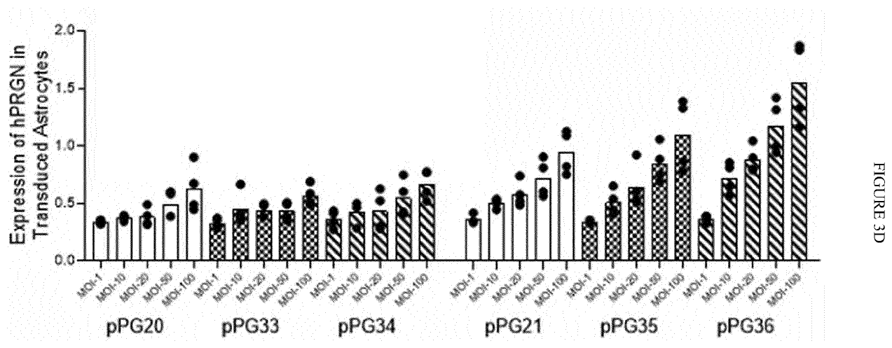

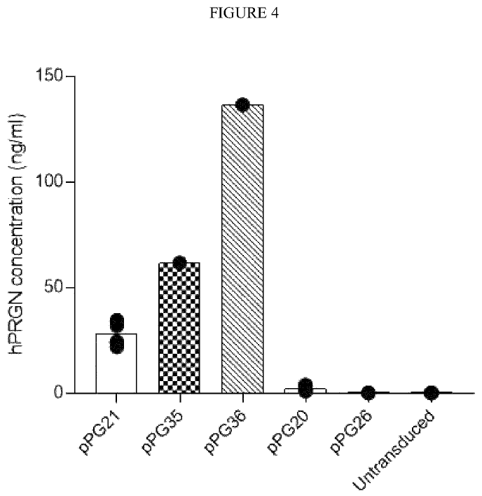

Wild-type mouse primary cortical neuron-astrocyte co-cultures were transduced to express human progranulin protein using lentivirus vector. Lentiviruses were applied at multiplicity of infection (MOIs) of 20. 10-days post lentiviral transduction, culture media was collected and ELISA performed. The results of this experiment are shown in FIG. 4. It was found that the constructs comprising engineered promoters MeCP2_1 and MeCP2_2 (pPG35 and pPG36, respectively) increased the secretion PGRN as compared to the construct comprising the minimal MeCP2 promoter (pPG21). Promoter MeCP2_2 (pPG36) provided the highest expression levels of secreted PGRN of all of the promoters tested.

### Example 5—PGRN Codon Optimization

Unmethylated CpG sites may induce innate immune responses meditated by toll-like receptor 9 (TLR9). Thus, codon-optimized nucleotide sequences encoding human PGRN which have a reduced CpG content were generated and cloned into expression vectors denoted CpG 0, 4, 9, 17, 25, 40, 71 and 90. Each of these vectors comprises a codon-optimized human PGRN nucleotide sequence having reduced CpG content relative to the corresponding WT sequence.

PGRN expression levels were evaluated for HAP-1 GRN knockout (GRN^{-/-}) cells transfected with the codon-optimized vectors and a WT vector were evaluated by ELISA and western blot. The expression level data are shown in FIG. 5. It was observed that the vector comprising the WT PGRN nucleotide sequence provided a higher level of PGRN expression than all of the codon-optimized vectors tested.

### Example 6—Expression of Human PGRN Corrects Lysosomal Deficit in GRN^{-/-} Mouse Primary Neurons

Western blot analysis was performed to quantify the level of lysosomal protein cathepsin D. Cathepsin D is a soluble lysosomal aspartic endopeptidase. The immature form is proteolytically cleaved yielding mature active lysosomal protease which is composed of heavy (˜30 kDa) and light (14 kDa) chains linked by non-covalent interactions. Cathepsin D is a marker of lysomosal dysfunction and an increased level of cathepsin D suggests impaired protein degradation and accumulation of autophagy cargos.

Mouse primary cortical neurons were prepared from WT or GRN^{-/-} (KO) mice. Three days post plating, neurons were transduced with lentiviral constructs (MOI of 20) to express human PGRN protein. Ten days post transduction, cells were harvested and protein extracted using RIPA buffer. The protein lysates were then subjected to western blotting to detect cathepsin D protein. Western blot levels of cathepsin D were normalized to the expression level of actin and GAPDH. Data are from three independent experiments.

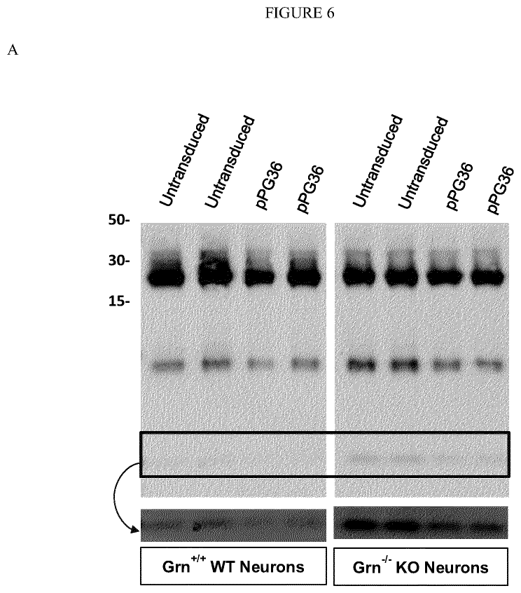

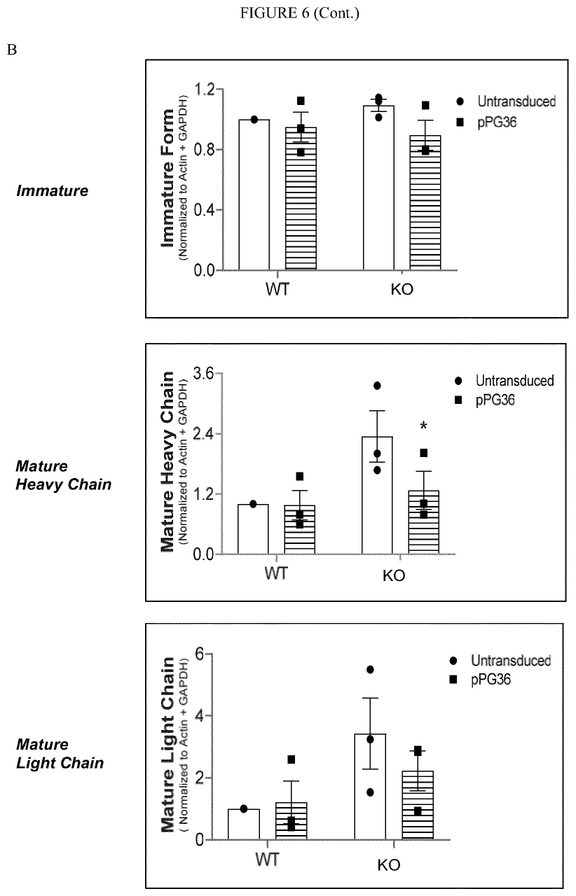

Compared to WT neurons, an increase in the level of mature cathepsin D was observed in KO neurons in untransduced conditions. Lentiviral-mediated expression of hPGRN (pPG36) prevented the maturation of cathepsin D. These results are presented in FIG. 6.

### Example 7—CNS Expression of Human PGRN (hPGRN) in WT and GRN^{-/-} Mice Following Striatal Injection of AAVTT-p1PG36

Striatum of adult (4 months old) WT or GRN^{-/-} mice were bilaterally injected with AAVTT-p1PG36 (AAVTT containing the construct of MeCP2_2 promoter+human PGRN transgene; SEQ ID NO: 18) or AAVTT-GFP (MeCP2_2 promoter+GFP transgene) or vehicle at a total dose of 2¹⁰vector genomes (vg). Animals were sacrificed after 4 weeks and CSF, plasma and brain tissue were collected and analyzed. Transcardial perfusion was performed with 1× PBS prior to dissection. Half of the brain was fixed for immunohistochemistry analysis, while other half was used for biochemical analysis (FRET). Different brain regions were dissected and frozen.

ELISA and FRET Analysis

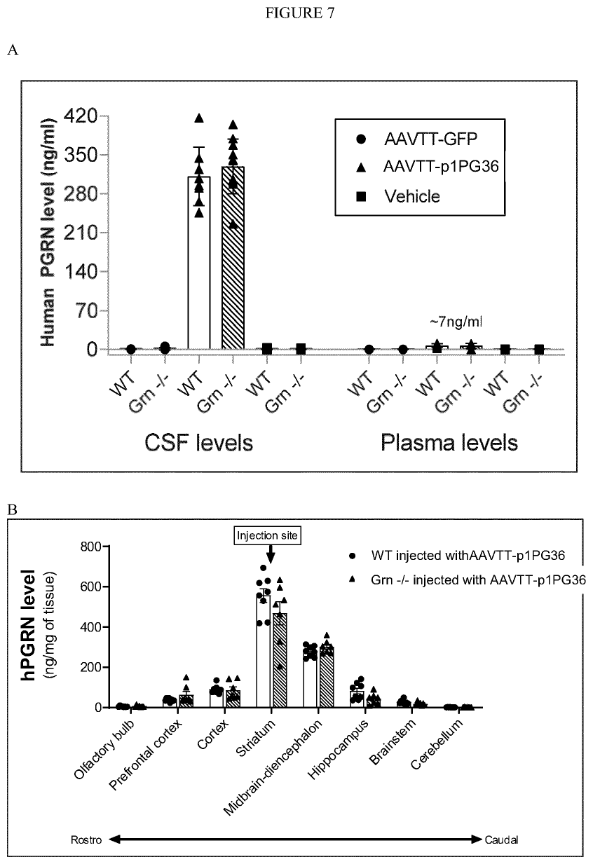

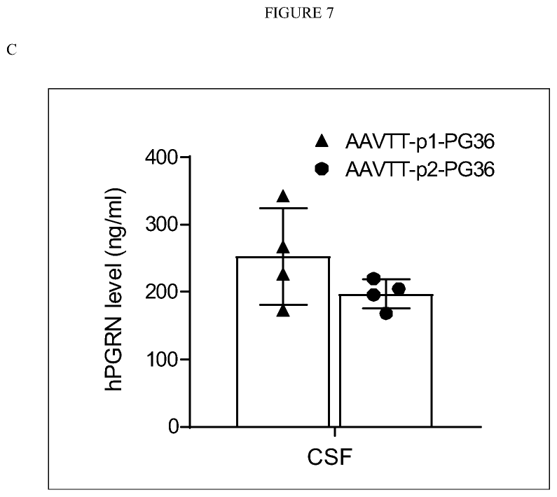

CSF and plasma levels of hPGRN (ng/ml) were measured using ELISA (Adipogen). A high level of hPGRN was detected in the CSF (1:100 dilution) of both WT and GRN-mice in animals injected with AAVTT-p1PG36 (SEQ ID NO: 18) and AAVTT-p2PG36 (SEQ ID NO: 19). These results are presented in FIGS. 7A and 7C. Low levels of hPGRN was also detected in the plasma of the mice (1:10 dilution). These results are presented in FIG. 7A.

FRET (Cisbio) was used to measure hPGRN concentration (ng/mg) in various brain regions of WT or GRN^{-/-} mice injected with AAVTT-p1PG36. Highest expression of hPGRN was detected close to the site of injection (Striatum and Mid-brain). Mid-levels of hPGRN expression was also detected in cortex and hippocampus. Low-levels of hPGRN expression were detected in distant brain regions such as brain-stem, olfactory bulb and cerebellum. These results are presented in FIG. 7B.

Immunohistochemistry

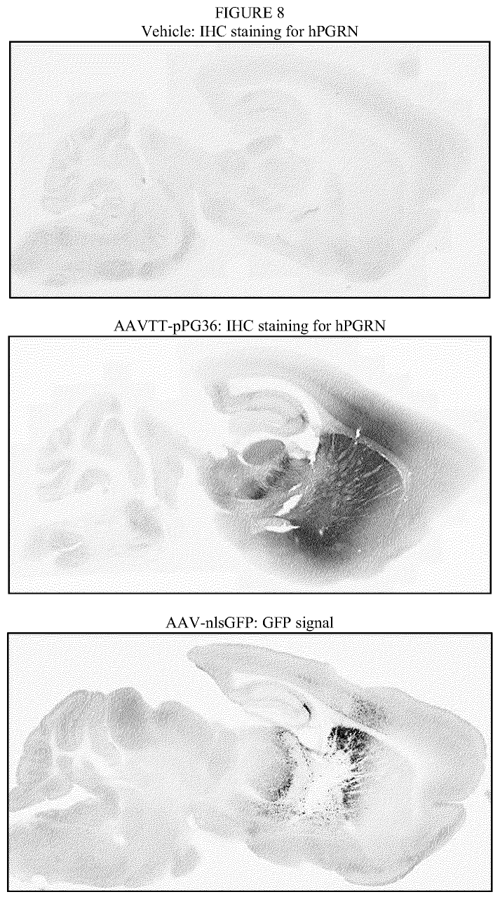

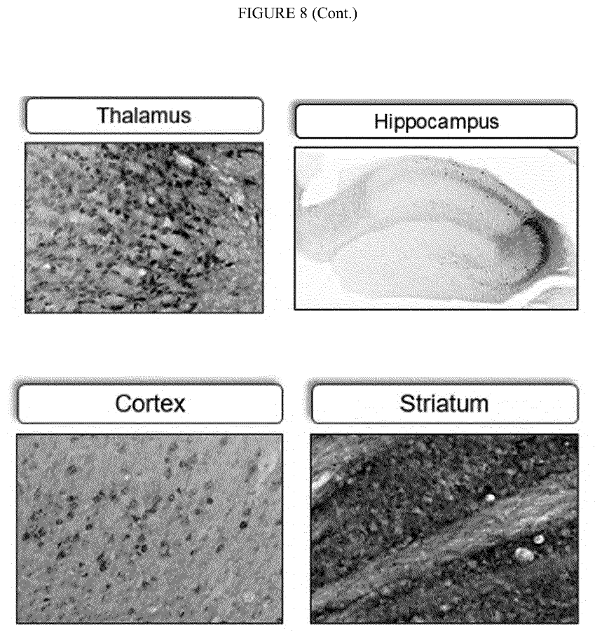

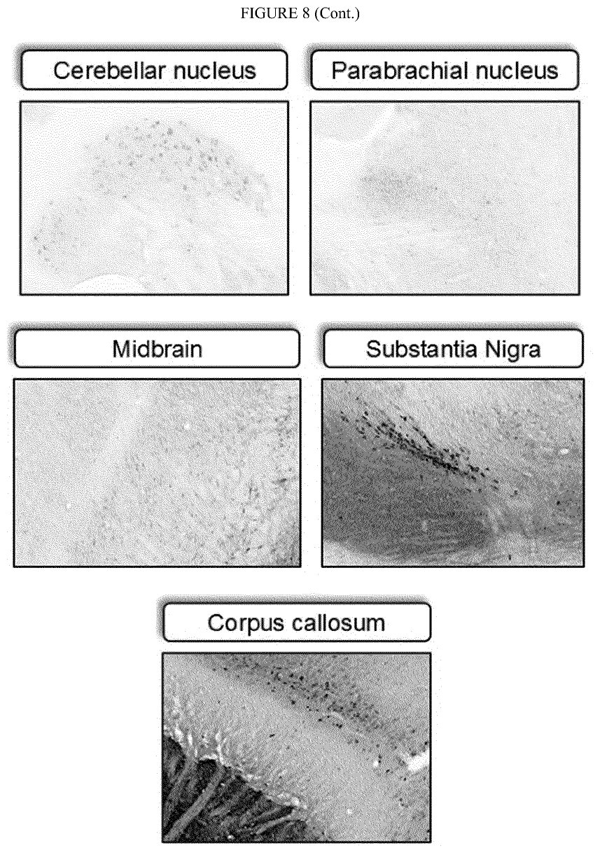

IHC staining of hPGRN was observed in brain of GRN^{-/-} KO mice that received intra-striatal administration of AAV-p1PG36 (SEQ ID NO: 18). Specific human PGRN signal was only detected in AAVTT-p1PG36 injected mice but not GFP injected mice. Similar to the FRET results, strong immunoreactivity was observed in the striatum of the mice where injection was performed. hPGRN immunoreactivity was also observed in brain regions away from the injection site, namely thalamus, mid-brain, substantia nigra, cortex and hippocampus. Cellular (cell-body) immunoreactivity was predominantly observed close to the site of injection, namely in the striatum, part of cortex, part of hippocampus, thalamus, mid-brain, suggesting transduction of cells with AAVTT-p1PG36 in these regions. Diffuse staining could be observed in most other brain regions, with a decrease in intensity from site of injection. This indicates that hPGRN is secreted in the extracellular space and gets diffused to distal brain regions via ISF and CSF flow. The results obtained for GRN^{-/-} mice are presented in FIG. 8, but similar images were obtained for the WT mice injected with AAV-p1PG36.

Immunofluorescence

It was shown that MeCP2 promoters were able to drive neuron-specific expression of hPGRN in vitro in primary mixed astrocyte-neuron culture (see Examples 3 and 4 above). In order to determine whether this neuronal-specificity is maintained in vivo in mice, double immunofluorescent labelling was performed on sections obtained from mice injected with AAVTT-p1PG36 (SEQ ID NO: 18) to label human PGRN and NeuN (neuronal marker) according to the following protocol (all steps performed at room temperature):

Sections were incubated with the neuronal marker NeuN (1:2,000; Abcam, ab177487) and human progranulin (1:1,000; R&D—AF2420) primary antibodies together, diluted in PBS containing 0.3% Triton X-100, overnight in a humidified chamber. Following incubation, the sections were washed three times with PBS, then incubated with the anti-rabbit Alexa 488 and anti-goat Alexa 647 secondary antibodies (all diluted at 1:1,000 in PBS; all from Thermo Fisher) for 1 hour. Sections were then counterstained with DAPI to label cell nuclei and washed three times with PBS. The sections were finally mounted with Prolong Gold anti-fade mounting media (Life Technologies) and a coverslip was applied. Digital images of stained sections were obtained using an AxioScan Z1 slide scanner with a 20×objective (Zeiss).

It was observed that almost all the cells expressing human PGRN in the cell body were NeuN positive. This demonstrates that AAVTT-p1PG36-mediated expression of hGRN is neuron specific in vivo. Neuronal expression of hGRN was observed in all various brain regions including striatum, cortex, hippocampus and thalamus. Importantly, no cellular expression hGRN was observed in the cell body of astrocytes (identified using GFAP staining) or microglia (identified using Iba1 staining). Thus, these in vivo data support the conclusion that MeCP2_2 promoter used is a neuron-specific promoter.

### Example 8—Human PGRN (hPGRN) Expression Impacts Cathepsin D Activity In Vivo in WT and GRN^{-/-} Mice Following Striatal Injection of AAVTT-p1PG36

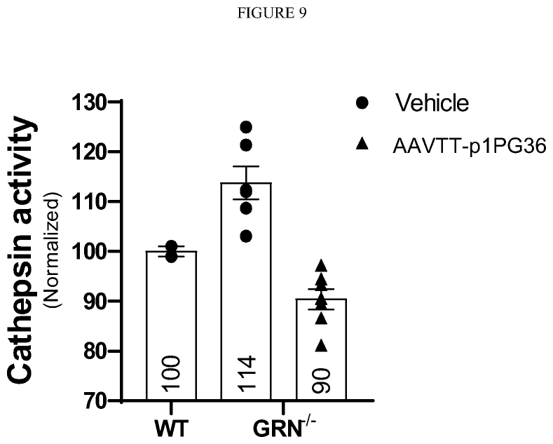

An increased level of cathepsin D suggests impaired protein degradation and accumulation of autophagy cargos. Cathepsin D enzymatic activity was measured in the mid-brain lysate of WT (GRN^{+/+}) mice treated with vehicle (as indicated by closed circles) and GRN^{-/-} KO mice treated with vehicle or AAVTT-p1PG36 (SEQ ID NO: 18). These results are presented in FIG. 9.

In vitro work revealed an accelerated maturation of cathepsin D maturation in GRN-primary neurons that was reversed by expression of pPG36 (see Example 6 above). Increased maturation is expected to be associated with an increase in Cathepsin D activity. A small increase in cathepsin D enzymatic activity in various brain regions of young (4-5-month) GRN^{-/-} mice compared to WT mice. Notably the increase in cathepsin D activity is more prominent in older animals (e.g. 1-year old). Thus, it is proposed that cathepsin D activity is an early marker of stress in GRN^{-/-} mice detectable as early as 4-5 months of age.

In any case, expression hGRN mediated by AAVTT-p1PG36 in 4-months old GRN-mice led to a reduction in cathepsin D enzymatic activity. This demonstrates that AAV-mediated hPGRN expression driven by the neuronal-specific MeCP2_2 promoter directly impacts cathepsin D activity (a marker of lysosomal dysfunction).

SEQUENCES

| SEQ | | |
|---|---|---|
| ID | | |
| NO | Description | Sequence |
| 1 | MeCP2 | AGCTGAATGGGGTCCGCCTCTTTTCCCTGC |
| | minimal | CTAAACAGACAGGAACTCCTGCCAATTGAG |
| | promoter | GGCGTCACCGCTAAGGCTCCGCCCCAGCCT |
| | | GGGCTCCACAACCAATGAAGGGTAATCTCG |
| | | ACAAAGAGCAAGGGGTGGGGCGCGGGCGCG |
| | | CAGGTGCAGCAGCACACAGGCTGGTCGGGA |
| | | GGGCGGGGCGCGACGTCTGCCGTGCGGGGT |
| | | CCCGGCATCGGTTGCGCGC |
| 2 | MeCP2_2 | gcgctccctcctctcggagagagggctgtg |
| | intron | gtaaaacccgtccggaaaTtggccgccgct |
| | | gccgccaccgccgccgccgccgccgcgccg |
| | | agcggaggaggaggaggaggcgaggaggag |
| | | agactgtgagtgggaccgccaaggccgcgg |
| | | gcggggacccttgctggggggcgggtaggg |
| | | gcgggacgtggcgcgggaggggcccgcggg |
| | | gtcggggacacggctggcggTtggcgtccc |
| | | tcctctctaccctccccctccctctgccgc |
| | | cggtggtggctttctccactcgtctcccgc |
| | | aatcgcgagcgacggttctcagcgcgatct |
| | | ccctggagccaccttcgaTtgacgccctcc |
| | | cgctgcccgccccatctgtgcgcatcctag |
| | | gccccagctgtgcaagcgcccttgtcgtct |
| | | gggcttcgccagttggggctgcgcgcgctc |
| | | ctgcccttcttggggctttgggcctcggca |
| | | ctgtcgcgcgcccgcggtcccggcctctcc |
| | | ctggatcgcgctgtccccttctccctcgcg |
| | | cgcccccactcccgttacttgctcccccct |
| | | cacacacacagactggcgcgcgtgcgcagt |
| | | ccatctcccgttgggagagtgcgccacaag |
| | | ggctcctgagctcttacccccatctctggg |
| | | ttttgctccctcctcctcctctcccattcc |
| | | gtgactttttgcccccactgcaagcgagtc |
| | | ggtccatcagctccattccccacttggcag |
| | | gaacaagttgagggttattgtccacccaca |
| | | aaaaggactagacattTtgttcctaggtcc |
| | | cacaactcatcataaagagTtggttgtagt |
| | | tctcatcaggaaccgtgggcaagggactgt |
| | | gcgttcctcagcactcgaagctcttccgtg |
| | | agaccTtgcccgcagggtgctctggttctt |
| | | tggggTtgctgtgctgtggcttcggaatTt |
| | | gagcgtcttcccaccctccctcccctccct |
| | | tcgccagcgttctgtctacaagaaagaata |
| | | ggcaggtgtccttggatatCgtagttgcta |
| | | atCgcctatacactgttctattacaccttt |
| | | ctgctaaggatagggtttttggttttggtt |
| | | ttggttttgttccccaccctccagtttggt |
| | | ttagttttggttttggcatttagggttttt |
| | | tgggggggagtaatatcttgtggtaaagac |
| | | ccatCtgacccaagataccttttttctcat |
| | | actggaaccctaggcagcagttgctatttc |
| | | cctgagttagcaatagttttacagtatttt |
| | | gaggccttttgtccataattctcacggaat |
| | | CcctcagggatCagattagctgctgttggg |
| | | atCaggaaattgggttacaccgctgaaatc |
| | | tcttgctggggcccttgttttgaattggaa |
| | | agtcaggaggctggaacgaaggctcacaag |
| | | ttaacagtgccagctgctcttccagaagcc |
| | | ctggattcagtcccaccaatccatCgcggg |
| | | tcacaaccatctgtaacttcagtcccaagg |
| | | ggtccgaAgccctcttctggctttgcccta |
| | | ttattttatttatcttatCtgtttttgtct |
| | | tgtcatCtggcaagcccagggggccattgg |
| | | gtgcaacttataaactgacttctgtatCtt |
| | | aagaagccaaccatacagtgcttacattcc |
| | | agaaaaaaaatCtgccactttaacagcact |
| | | agaactagggtttagagaagtatCataaag |
| | | gtcaaatatCtttgaccaatatcaccagca |
| | | acctaaagctgttaagaaatctttgggccc |
| | | cagcttgacccaaggatacagtatcctagg |
| | | gaagttaccaaaatcagagatagtatgcag |
| | | cagccaggggtctcatgtgtggcactcaag |
| | | ctcacctatactcactactgtgcagacagc |
| | | tgtgttctctgtaatacttacatatttgtt |
| | | taatacttcagggaggaaaagtcagaagac |
| | | caggatctccagggcctca |
| 3 | MeCP2_2 | AGCTGAATGGGGTCCGCCTCTTTTCCCTGC |
| | promoter | CTAAACAGACAGGAACTCCTGCCAATTGAG |
| | | GGCGTCACCGCTAAGGCTCCGCCCCAGCCT |
| | | GGGCTCCACAACCAATGAAGGGTAATCTCG |
| | | ACAAAGAGCAAGGGGTGGGGCGCGGGCGCG |
| | | CAGGTGCAGCAGCACACAGGCTGGTCGGGA |
| | | GGGCGGGGCGCGACGTCTGCCGTGCGGGGT |
| | | CCCGGCATCGGTTGCGCGCACCGGTgcgct |
| | | ccctcctctcggagagagggctgtggtaaa |
| | | acccgtccggaaaTtggccgccgctgccgc |
| | | caccgccgccgccgccgccgcgccgagcgg |
| | | aggaggaggaggaggcgaggaggagagact |
| | | gtgagtgggaccgccaaggccgcgggcggg |
| | | gacccttgctggggggcgggtaggggcggg |
| | | acgtggcgcgggaggggcccgcggggtcgg |
| | | gcgacacggctggcggTtggcgtccctcct |
| | | ctctaccctccccctccctctgccgccggt |
| | | ggtggctttctccactcgtctcccgcaatc |
| | | gcgagcgacggttctcagcgcgatctccct |
| | | ggagccaccttcgaTtgacgccctcccgct |
| | | gcccgccccatctgtgcgcatcctaggccc |
| | | cagctgtgcaagcgcccttgtcgtctgggc |
| | | ttcgccagttggggctgcgcgcgctcctgc |
| | | ccttcttggggctttgggcctcggcactgt |
| | | cgcgcgcccgcggtcccggcctctccctgg |
| | | atcgcgctgtccccttctccctcgcgcgcc |
| | | cccactcccgttacttgctcccccctcaca |
| | | cacacagactggcgcgcgtgcgcagtccat |
| | | ctcccgttgggagagtgcgccacaagggct |
| | | cctgagctcttacccccatctctgggtttt |
| | | gctccctcctcctcctctcccattccgtga |
| | | ctttttgcccccactgcaagcgagtcggtc |
| | | catcagctccattccccacttggcaggaac |
| | | aagttgagggttattgtccacccacaaaaa |
| | | ggactagacattTtgttcctaggtcccaca |
| | | actcatcataaagagTtggttgtagttctc |
| | | atcaggaaccgtgggcaagggactgtgcgt |
| | | tcctcagcactcgaagctcttccgtgagac |
| | | cTtgcccgcagggtgctctggttctttggg |
| | | gTtgctgtgctgtggcttcggaatTtgagc |
| | | gtcttcccaccctccctcccctcccttcgc |
| | | cagcgttctgtctacaagaaagaataggca |
| | | ggtgtccttggatatCgtagttgctaatCg |
| | | cctatacactgttctattacacctttctgc |
| | | taaggatagggtttttggttttggttttgg |
| | | ttttgttccccaccctccagtttggtttag |
| | | ttttggttttggcatttaggtttttctcat |
| | | actggaaccctaggcagcagttgctatttc |
| | | cctgagttagcaatagttttacagtatttt |
| | | gaggccttttgtccataattctcacggaat |
| | | CcctcagggatCagattagctgctgttggg |
| | | atCaggaaattgggttacaccgctgaaatc |
| | | tcttgctgcagtgccagctgctcttccaga |
| | | agccctggattcagtcccaccaatccatCg |
| | | cgggtcacaaccatctgtaacttcagtccc |
| | | aaggggtccgaAgccctcttctggctttgc |
| | | cctattattttatttatcttatCtgttttt |
| | | gtcttgtcatCtggcaagcccagggggcca |
| | | ttgggtgcaacttataaactgacttctgta |
| | | tCttaagaagccaaccatacagtgcttaca |
| | | ttccagaaaaaaaatCtgccactttaacag |
| | | cactagaactagggtttagagaagtatCat |
| | | aaaggtcaaatatCtttgaccaatatcacc |
| | | agcaacctaaagctgttaagaaatctttgg |
| | | gccccagcttgacccaaggatacagtatcc |
| | | tagggaagttaccaaaatcagagatagtat |
| | | gcagcagccaggggtctcatgtgtggcact |
| | | caagctcacctatactcactactgtgcaga |
| | | cagctgtgttctctgtaatacttacatatt |
| | | tgtttaatacttcagggaggaaaagtcaga |
| | | agaccaggatctccagggcctca |
| 4 | MeCP2_2 | gcgctccctcctctcggagagagggctgtg |
| | intron- | gtaaaacccgtccggaaaTtggccgccgct |
| | exon1 | gccgccaccgccgccgccgccgccgcgccg |
| | | agcggaggaggaggaggaggcgaggaggag |
| | | agact |
| 5 | MeCP2_2 | gtgagtgggaccgccaaggccgcgggcggg |
| | intron-5′ | gacccttgctgggggggggtaggggtccct |
| | intron | cctctctaccctccccctccctctgccgcc |
| | | ggtggtggctttctccactcgtctcccgca |
| | | atcgcgagcgacggttctcagcgcgatctc |
| | | cctggagccaccttcgaTtgacgccctccc |
| | | gctgcccgccccatctgtgcgcatcctagg |
| | | ccccagctgtgcaAgcgcccttgtcgtctg |
| | | ggcttcgccagttggggctgcgcgcgctcc |
| | | tgcccttctcgcgctgtccccttctccctc |
| | | gcgcgcccccactcccgttacttgctcccc |
| | | cctcacacacacagactggcgcgcgtgcgc |
| | | agtccatctcccgttgggagagtgcgccac |
| | | aagggctcctgagctcttacccccatctct |
| | | gggttttgctccctcctcctcctctcccat |
| | | tccgtgactttttgcccccactgcaagcga |
| | | gtcggtccatcagctccattccccacttgg |
| | | caggaacaagttgagggttattgtccaccc |
| | | acaaaaaggactagacattTtgttcctagg |
| | | tcccacaactcatcataaagagTtggttgt |
| | | agttctcatcaggaagtcttcccaccctcc |
| | | ctcccctcccttcgccagcg |
| 6 | MeCP2_2 | ttctgtctacaagaaagaataggcaggtgt |
| | intron-3′ | ccttggatatCgtagttgctaatCgcctat |
| | intron | acactgttctattacacctttctgctaagg |
| | | atagggtttttggttttggttttggttttg |
| | | ttccccaccctccagtttggtttagttttg |
| | | gttttggcatttagggttttctcatactgg |
| | | aaccctaggcagcagttgctatttccctga |
| | | gttagcaatagttttacagtattttgaggc |
| | | cttttgtccataattctcacggaatCcctc |
| | | agggatCagattagctgctgttgggatCag |
| | | gaaattgggttacaccgctgaaatctcttg |
| | | ctggggcccttgttttgaattggaaagtca |
| | | ggaggctggaacgaaggctcacaagttaac |
| | | agtgccagctgctcttccagaagccctgga |
| | | ttcagtcccaccaatccatCgcgggtcaca |
| | | accatctgtaacttcagtcccaaggggtcc |
| | | gaAgccctcttctggctttgccctattatt |
| | | ttatttatcttatCtgtttttgtcttgtca |
| | | tCtggcaagcccagggggcattgggtgcaa |
| | | cttataaactgacttctgtatCttaagaag |
| | | ccaaccatacagtgcttacattccagaaaa |
| | | aaaatCtgccactttaacagcactagaact |
| | | agggtttagagaagtatCataaaggtcaaa |
| | | tatCtttgaccaatatcaccagcaacctaa |
| | | agctgttaagaaatctttgggccccagctt |
| | | gacccaaggatacagtatcctagggaagtt |
| | | accaaaatcagagatagtatgcagcagcca |
| | | ggggtctcatgtgtggcactcaagctcacc |
| | | tatactcactactgtgcagacagctgtgtt |
| | | ctctgtaatacttacatattttttaatact |
| | | tcag |
| 7 | MeCP2_2 | ggaggaaaagtcagaagaccaggatctcca |
| | intron- | gggcctca |
| | exon2 | |
| 8 | MeCP2 1 | CTCTACCATTACGTTTTATCCTCAGACTCT |
| | promoter | ATCTCCCCATTTTAAAGGAATATTATTTTT |
| | | AAATGCTACACTCTCATTTTTTAAATGGCT |
| | | CCTTTTAATTCTACTGCTAAAATACTTTTG |
| | | GTACAATATGCCTTTTTTTCTATTTTTTTT |
| | | TTTTAGTGCAAGTATAAAATATGTCATTTA |
| | | AATCTCTTTTATCTAATTTAAGGAACTTAA |
| | | GATTTTCTTCCCAAAATTTCACAAGGTAGG |
| | | AAAATGATGTACTTTATTTTTGTGAATGAT |
| | | ATTGCACTGTAGATCTTGCTGTTTCTTGCT |
| | | TTGTTCTCAATTAAATATCATGTTTCCTCT |
| | | ACAGCTTTATAGACATTTTGATCAGATTAA |
| | | GGACATTCTAATTATAGTTCTTTAAGGTGT |
| | | TTTTAAAATCATATGTAGGTATTGAATTTT |
| | | ATTAAATGCTTTTTCCTGCATGTATTGGGA |
| | | TACTCACATGATCTGTTCTTAAAATTATAA |
| | | TTGATTTCTAATTTTAAACCATCCTTGCAT |
| | | TCGTGGAATAAAACTCAGCCTGAAGGTGTG |
| | | GCTGGAGAGATGGCTTGTTGCTCTTGCAGA |
| | | GGACCCAAGTTCAGATCCTATTCTCTGTAT |
| | | ATGCAAATACCTGTATTCTCACACCCCAAC |
| | | ATACACACACACACACACACACACACACAC |
| | | ACACACTACCACTCTTACCCACTTGTTTTT |
| | | TACATTTCTATCTTAGTATTTTATGTGTAT |
| | | AAGTGTTTTGCCTGGTGCTCACTGTGGTCA |
| | | GAAGAGGGCACAGGATTCCCTGAGACTAGA |
| | | ATTACAAATGGTTTAGAATGGCCATATGGG |
| | | AAATCCTCTAGATTTCCCCCACTGTAGTAA |
| | | GATATTCACTTAGGTGATCCTGTCCCAAAG |
| | | TCAGCCATCATCCTTATTTTTTTTCTTTCT |
| | | TTCCATCTGATATCCAATACTTTTGGCTCA |
| | | ATTTTTAACATAAATCTTAACTATCACAAC |
| | | TTATCAGATTTCAACTGCTACTGTCCTGGT |
| | | TAAAGCCTTCATCATCTATCTTTCTTCAAC |
| | | TGCTGCCAGGACCTCTGGACCAGCCAGTTC |
| | | TTCATTCTTCACTGGCAACATAGGTTTTAT |
| | | GGTGACAGCTAGTGACTCAAATATTTATCA |
| | | AGGGCTTCTCATCTCAAAATAATCTCCTAG |
| | | TTCTTTTGGTGGCCTAGGTCTCTCTCCAGT |
| | | CACACTGGCCTCCTTAGTAAGGCAGGCATA |
| | | GTCCTTCCTTAGAGTGTTTAAACTTGCCTA |
| | | GAATGTTTTCCCCAATTACCCATATTGGGA |
| | | GACGACATGAGGGCAAAAGCTAGAGGGTAT |
| | | CATAATAGCACTTCTTTTGTCCTTGCCCTA |
| | | TCTATTTCAAAGTCTTTATCTCTGTGCAAA |
| | | ATTTTAAGTTCTACTTTCTTGTATGTTTAG |
| | | TATGACTCTTCCTTACCAGGAGTCTAGTTT |
| | | GTCTCCTTGTTCAGTACTAAAACAGTGCCT |
| | | AGCAAATAAATGAATAGAGAGGGGAGCCAA |
| | | ATTTGAATCAGAAAGTCTCTTGTTGCATAG |
| | | TGTTTAAAAAACAAACAAAGAAAGAAAGTC |
| | | TCTTGTTGAGCATTTGTTTAGCACAAAGAG |
| | | CATTGGATGCTGACTGGTATCAGGGTAAGG |
| | | CTGCTTTGACAATGCTCCCTCTGGCCTCAC |
| | | TCCCTTTTATACGTACTTCCATCAAACCAT |
| | | CTGATTCAACAATGACAGACCGATCTCTTA |
| | | TGGGCTTGGCACACACCATCTGCCCATTAT |
| | | AAACGTCTGCAAAGACCAAGGTTTGATATG |
| | | TTGATTTTACTGTCAGCCTTAAGAGTGCGA |
| | | CATCTGCTAATTTAGTGTAATAATACAATC |
| | | AGTAGACCCTTTAAAACAAGTCCCTTGGCT |
| | | TGGAACAACGCCAGGCTCCTCAACAGGCAA |
| | | CTTTGCTACTTCTACAGAAAATGATAATAA |
| | | AGAAATGCTGGTGAAGTCAAATGCTTATCA |
| | | CAATGGTGAACTACTCAGCAGGGAGGCTCT |
| | | AATAGGCGCCAAGAGCCTAGACTTCCTTAA |
| | | GCGCCAGAGTCCACAAGGGCCCAGTTAATC |
| | | CTCAACATTCAAATGCTGCCCACAAAACCA |
| | | GCCCCTCTGTGCCCTAGCCGCCTCTTTTTT |
| | | CCAAGTGACAGTAGAACTCCACCAATCCGC |
| | | TTAATTAAAGCTGAATGGGGTCCGCCTCTT |
| | | TTCCCTGCCTAAACAGACAGGAACTCCTGC |
| | | CAATTGAGGGCGTCACCGCTAAGGCTCCGC |
| | | CCCAGCCTGGGCTCCACAACCAATGAAGGG |
| | | TAATCTCGACAAAGAGCAAGGGGTGGGGCG |
| | | CGGGCGCGCAGGTGCAGCAGCACACAGGCT |
| | | GGTCGGGAGGGCGGGGCGCGACGTCTGCCG |
| | | TGCGGGGTCCCGGCATCGGTTGCGCGC |
| 9 | MeCP2 1 | CTCTACCATTACGTTTTATCCTCAGACTCT |
| | intron | ATCTCCCCATTTTAAAGGAATATTATTTTT |
| | | AAATGCTACACTCTCATTTTTTAAATGGCT |
| | | CCTTTTAATTCTACTGCTAAAATACTTTTG |
| | | GTACAATATGCCTTTTTTTCTATTTTTTTT |
| | | TTTTAGTGCAAGTATAAAATATGTCATTTA |
| | | AATCTCTTTTATCTAATTTAAGGAACTTAA |
| | | GATTTTCTTCCCAAAATTTCACAAGGTAGG |
| | | AAAATGATGTACTTTATTTTTGTGAATGAT |
| | | ATTGCACTGTAGATCTTGCTGTTTCTTGCT |
| | | TTGTTCTCAATTAAATATCATGTTTCCTCT |
| | | ACAGCTTTATAGACATTTTGATCAGATTAA |
| | | GGACATTCTAATTATAGTTCTTTAAGGTGT |
| | | TTTTAAAATCATATGTAGGTATTGAATTTT |
| | | ATTAAATGCTTTTTCCTGCATGTATTGGGA |
| | | TACTCACATGATCTGTTCTTAAAATTATAA |
| | | TTGATTTCTAATTTTAAACCATCCTTGCAT |
| | | TCGTGGAATAAAACTCAGCCTGAAGGTGTG |
| | | GCTGGAGAGATGGCTTGTTGCTCTTGCAGA |
| | | GGACCCAAGTTCAGATCCTATTCTCTGTAT |
| | | ATGCAAATACCTGTATTCTCACACCCCAAC |
| | | ATACACACACACACACACACACACACACAC |
| | | ACACACTACCACTCTTACCCACTTGTTTTT |
| | | TACATTTCTATCTTAGTATTTTATGTGTAT |
| | | AAGTGTTTTGCCTGGTGCTCACTGTGGTCA |
| | | GAAGAGGGCACAGGATTCCCTGAGACTAGA |
| | | ATTACAAATGGTTTAGAATGGCCATATGGG |
| | | AAATCCTCTAGATTTCCCCCACTGTAGTAA |
| | | GATATTCACTTAGGTGATCCTGTCCCAAAG |
| | | TCAGCCATCATCCTTATTTTTTTTCTTTCT |
| | | TTCCATCTGATATCCAATACTTTTGGCTCA |
| | | ATTTTTAACATAAATCTTAACTATCACAAC |
| | | TTATCAGATTTCAACTGCTACTGTCCTGGT |
| | | TAAAGCCTTCATCATCTATCTTTCTTCAAC |
| | | TGCTGCCAGGACCTCTGGACCAGCCAGTTC |
| | | TTCATTCTTCACTGGCAACATAGGTITTAT |
| | | GGTGACAGCTAGTGACTCAAATATTTATCA |
| | | AGGGCTTCTCATCTCAAAATAATCTCCTAG |
| | | TTCTTTTGGTGGCCTAGGTCTCTCTCCAGT |
| | | CACACTGGCCTCCTTAGTAAGGCAGGCATA |
| | | GTCCTTCCTTAGAGTGTTTAAACTTGCCTA |
| | | GAATGTTTTCCCCAATTACCCATATTGGGA |
| | | GACGACATGAGGGCAAAAGCTAGAGGGTAT |
| | | CATAATAGCACTTCTTTTGTCCTTGCCCTA |
| | | TCTATTTCAAAGTCTTTATCTCTGTGCAAA |
| | | ATTTTAAGTTCTACTTTCTTGTATGTTTAG |
| | | TATGACTCTTCCTTACCAGGAGTCTAGTTT |
| | | GTCTCCTTGTTCAGTACTAAAACAGTGCCT |
| | | AGCAAATAAATGAATAGAGAGGGGAGCCAA |
| | | ATTTGAATCAGAAAGTCTCTTGTTGCATAG |
| | | TGTTTAAAAAACAAACAAAGAAAGAAAGTC |
| | | TCTTGTTGAGCATTTGTTTAGCACAAAGAG |
| | | CATTGGATGCTGACTGGTATCAGGGTAAGG |
| | | CTGCTTTGACAATGCTCCCTCTGGCCTCAC |
| | | TCCCTTTTATACGTACTTCCATCAAACCAT |
| | | CTGATTCAACAATGACAGACCGATCTCTTA |
| | | TGGGCTTGGCACACACCATCTGCCCATTAT |
| | | AAACGTCTGCAAAGACCAAGGTTTGATATG |
| | | TTGATTTTACTGTCAGCCTTAAGAGTGCGA |
| | | CATCTGCTAATTTAGTGTAATAATACAATC |
| | | AGTAGACCCTTTAAAACAAGTCCCTTGGCT |
| | | TGGAACAACGCCAGGCTCCTCAACAGGCAA |
| | | CTTTGCTACTTCTACAGAAAATGATAATAA |
| | | AGAAATGCTGGTGAAGTCAAATGCTTATCA |
| | | CAATGGTGAACTACTCAGCAGGGAGGCTCT |
| | | AATAGGCGCCAAGAGCCTAGACTTCCTTAA |
| | | GCGCCAGAGTCCACAAGGGCCCAGTTAATC |
| | | CTCAACATTCAAATGCTGCCCACAAAACCA |
| | | GCCCCTCTGTGCCCTAGCCGCCTCTTTTTT |
| | | CCAAGTGACAGTAGAACTCCACCAATCCGC |
| | | TTAATTAA |
| 10 | pPG35 | GGCTGTGACCAGCACACCAGCTGCCCGGTG |
| | | GGGCAGACCTGCTGCCCGAGCCTGGGTGGG |
| | | AGCTGGGCCTGCTGCCAGTTGCCCCATGCT |
| | | GTGTGCTGCGAGGATCGCCAGCACTGCTGC |
| | | CCGGCTGGCTACACCTGCAACGTGAAGGCT |
| | | CGATCCTGCGAGAAGGAAGTGGTCTCTGCC |
| | | CAGCCTGCCACCTTCCTGGCCCGTAGCCCT |
| | | CACGTGGGTGTGAAGGACGTGGAGTGTGGG |
| | | GAAGGACACTTCTGCCATGATAACCAGACC |
| | | TGCTGCCGAGACAACCGACAGGGCTGGGCC |
| | | TGCTGTCCCTACCGCCAGGGCGTCTGTTGT |
| | | GCTGATCGGCGCCACTGCTGTCCTGCTGGC |
| | | TTCCGCTGCGCAGCCAGGGGTACCAAGTGT |
| | | TTGCGCAGGGAGGCCCCGCGCTGGGACGCC |
| | | CCTTTGAGGGACCCAGCCTTGAGACAGCTG |
| | | CTGTGAGGCCAggcCGGCCGAATTCGATAT |
| | | CAAGCTTATCGATAATCAACCTCTGGATTA |
| | | CAAAATTTGTGAAAGATTGACTGGTATTCT |
| | | TAACTATGTTGCTCCTTTTACGCTATGTGG |
| | | ATACGCTGCTTTAATGCCTTTGTATCATGC |
| | | TATTGCTTCCCGTATGGCTTTCATTTTCTC |
| | | CTCCTTGTATAAATCCTGGTTGCTGTCTCT |
| | | TTATGAGGAGTTGTGGCCCGTTGTCAGGCA |
| | | ACGTGGCGTGGTGTGCACTGTGTTTGCTGA |
| | | CGCAACCCCCACTGGTTGGGGCATTGCCAC |
| | | CACCTGTCAGCTCCTTTCCGGGACTTTCGC |
| | | TTTCCCCCTCCCTATTGCCACGGCGGAACT |
| | | CATCGCCGCCTGCCTTGCCCGCTGCTGGAC |
| | | AGGGGCTCGGCTGTTGGGCACTGACAATTC |
| | | CGTGGTGTTGTCGGGGAAATCATCGTCCTT |
| | | TCCTTGGCTGCTCGCCTGTGTTGCCACCTG |
| | | GATTCTGCGCGGGACGTCCTTCTGCTACGT |
| | | CCCTTCGGCCCTCAATCCAGCGGACCTTCC |
| | | TTCCCGCGGCCTGCTGCCGGCTCTGCGGCC |
| | | TCTTCCGCGTCTTCGCCTTCGCCCTCAGAC |
| | | GAGTCGGATCTCCCTTTGGGCCGCCTCCCC |
| | | GCATCGATACCGTCGACCTCGAGACCTAGA |
| | | AAAACATGGAGCAATCACAAGTAGCAATAC |
| | | AGCAGCTACCAATGCTGATTGTGCCTGGCT |
| | | AGAAGCACAAGAGGAGGAGGAGGTGGGTTT |
| | | TCCAGTCACACCTCAGGTACCTTTAAGACC |
| | | AATGACTTACAAGGCAGCTGTAGATCTTAG |
| | | CCACTTTTTAAAAGAAAAGGGGGGACTGGA |
| | | AGGGCTAATTCACTCCCAACGAAGACAAGA |
| | | TATCCTTGATCTGTGGATCTACCACACACA |
| | | AGGCTACTTCCCTGATTGGCAGAACTACAC |
| | | ACCAGGGCCAGGGATCAGATATCCACTGAC |
| | | CTTTGGATGGTGCTACAAGCTAGTACCAGT |
| | | TGAGCAAGAGAAGGTAGAAGAAGCCAATGA |
| | | AGGAGAGAACACCCGCTTGTTACACCCTGT |
| | | GAGCCTGCATGGGATGGATGACCCGGAGAG |
| | | AGAAGTATTAGAGTGGAGGTTTGACAGCCG |
| | | CCTAGCATTTCATCACATGGCCCGAGAGCT |
| | | GCATCCGGACTGTACTGGGTCTCTCTGGTT |
| | | AGACCAGATCTGAGCCTGGGAGCTCTCTGG |
| | | CTAACTAGGGAACCCACTGCTTAAGCCTCA |
| | | ATAAAGCTTGCCTTGAGTGCTTCAAGTAGT |
| | | GTGTGCCCGTCTGTTGTGTGACTCTGGTAA |
| | | CTAGAGATCCCTCAGACCCTTTTAGTCAGT |
| | | GTGGAAAATCTCTAGCAGGGCCCGTTTAAA |
| | | CCCGCTGATCAGCCTCGACTGTGCCTTCTA |
| | | GTTGCCAGCCATCTGTTGTTTGCCCCTCCC |
| | | CCGTGCCTTCCTTGACCCTGGAAGGTGCCA |
| | | CTCCCACTGTCCTTTCCTAATAAAATGAGG |
| | | AAATTGCATCGCATTGTCTGAGTAGGTGTC |
| | | ATTCTATTCTGGGGGGTGGGGTGGGGCAGG |
| | | ACAGCAAGGGGGAGGATTGGGAAGACAATA |
| | | GCAGGCATGCTGGGGATGCGGTGGGCTCTA |
| | | TGGCTTCTGAGGCGGAAAGAACCAGCTGGG |
| | | GCTCTAGGGGGTATCCCCACGCGCCCTGTA |
| | | GCGGCGCATTAAGCGCGGCGGGTGTGGTGG |
| | | TTACGCGCAGCGTGACCGCTACACTTGCCA |
| | | GCGCCCTAGCGCCCGCTCCTTTCGCTTTCT |
| | | TCCCTTCCTTTCTCGCCACGTTCGCCGGCT |
| | | TTCCCCGTCAAGCTCTAAATCGGGGGCTCC |
| | | CTTTAGGGTTCCGATTTAGTGCTTTACGGC |
| | | ACCTCGACCCCAAAAAACTTGATTAGGGTG |
| | | ATGGTTCACGTAGTGGGCCATCGCCCTGAT |
| | | AGACGGTTTTTCGCCCTTTGACGTTGGAGT |
| | | CCACGTTCTTTAATAGTGGACTCTTGTTCC |
| | | AAACTGGAACAACACTCAACCCTATCTCGG |
| | | TCTATTCTTTTGATTTATAAGGGATTTTGC |
| | | CGATTTCGGCCTATTGGTTAAAAAATGAGC |
| | | TGATTTAACAAAAATTTAACGCGAATTAAT |
| | | TCTGTGGAATGTGTGTCAGTTAGGGTGTGG |
| | | AAAGTCCCCAGGCTCCCCAGCAGGCAGAAG |
| | | TATGCAAAGCATGCATCTCAATTAGTCAGC |
| | | AACCAGGTGTGGAAAGTCCCCAGGCTCCCC |
| | | AGCAGGCAGAAGTATGCAAAGCATGCATCT |
| | | CAATTAGTCAGCAACCATAGTCCCGCCCCT |
| | | AACTCCGCCCATCCCGCCCCTAACTCCGCC |
| | | CAGTTCCGCCCATTCTCCGCCCCATGGCTG |
| | | ACTAATTTTTTTTATTTATGCAGAGGCCGA |
| | | GGCCGCCTCTGCCTCTGAGCTATTCCAGAA |
| | | GTAGTGAGGAGGCTTTTTTGGAGGCCTAGG |
| | | CTTTTGCAAAAAGCTCCCGGGAGCTTGTAT |
| | | ATCCATTTTCGGATCTGATCAGCACGTGTT |
| | | GACAATTAATCATCGGCATAGTATATCGGC |
| | | ATAGTATAATACGACAAGGTGAGGAACTAA |
| | | ACCATGGCCAAGTTGACCAGTGCCGTTCCG |
| | | GTGCTCACCGCGCGCGACGTCGCCGGAGCG |
| | | GTCGAGTTCTGGACCGACCGGCTCGGGTTC |
| | | TCCCGGGACTTCGTGGAGGACGACTTCGCC |
| | | GGTGTGGTCCGGGACGACGTGACCCTGTTC |
| | | ATCAGCGCGGTCCAGGACCAGGTGGTGCCG |
| | | GACAACACCCTGGCCTGGGTGTGGGTGCGC |
| | | GGCCTGGACGAGCTGTACGCCGAGTGGTCG |
| | | GAGGTCGTGTCCACGAACTTCCGGGACGCC |
| | | TCCGGGCCGGCCATGACCGAGATCGGCGAG |
| | | CAGCCGTGGGGGCGGGAGTTCGCCCTGCGC |
| | | GACCCGGCCGGCAACTGCGTGCACTTCGTG |
| | | GCCGAGGAGCAGGACTGACACGTGCTACGA |
| | | GATTTCGATTCCACCGCCGCCTTCTATGAA |
| | | AGGTTGGGCTTCGGAATCGTTTTCCGGGAC |
| | | GCCGGCTGGATGATCCTCCAGCGCGGGGAT |
| | | CTCATGCTGGAGTTCTTCGCCCACCCCAAC |
| | | TTGTTTATTGCAGCTTATAATGGTTACAAA |
| | | TAAAGCAATAGCATCACAAATTTCACAAAT |
| | | AAAGCATTTTTTTCACTGCATTCTAGTTGT |
| | | GGTTTGTCCAAACTCATCAATGTATCTTAT |
| | | CATGTCTGTATACCGTCGACCTCTAGCTAG |
| | | AGCTTGGCGTAATCATGGTCATAGCTGTTT |
| | | CCTGTGTGAAATTGTTATCCGCTCACAATT |
| | | CCACACAACATACGAGCCGGAAGCATAAAG |
| | | TGTAAAGCCTGGGGTGCCTAATGAGTGAGC |
| | | TAACTCACATTAATTGCGTTGCGCTCACTG |
| | | CCCGCTTTCCAGTCGGGAAACCTGTCGTGC |
| | | CAGCTGCATTAATGAATCGGCCAACGCGCG |
| | | GGGAGAGGCGGTTTGCGTATTGGGCGCTCT |
| | | TCCGCTTCCTCGCTCACTGACTCGCTGCGC |
| | | TCGGTCGTTCGGCTGCGGCGAGCGGTATCA |
| | | GCTCACTCAAAGGCGGTAATACGGTTATCC |
| | | ACAGAATCAGGGGATAACGCAGGAAAGAAC |
| | | ATGTGAGCAAAAGGCCAGCAAAAGGCCAGG |
| | | AACCGTAAAAAGGCCGCGTTGCTGGCGTTT |
| | | TTCCATAGGCTCCGCCCCCCTGACGAGCAT |
| | | CACAAAAATCGACGCTCAAGTCAGAGGTGG |
| | | CGAAACCCGACAGGACTATAAAGATACCAG |
| | | GCGTTTCCCCCTGGAAGCTCCCTCGTGCGC |
| | | TCTCCTGTTCCGACCCTGCCGCTTACCGGA |
| | | TACCTGTCCGCCTTTCTCCCTTCGGGAAGC |
| | | GTGGCGCTTTCTCATAGCTCACGCTGTAGG |
| | | TATCTCAGTTCGGTGTAGGTCGTTCGCTCC |
| | | AAGCTGGGCTGTGTGCACGAACCCCCCGTT |
| | | CAGCCCGACCGCTGCGCCTTATCCGGTAAC |
| | | TATCGTCTTGAGTCCAACCCGGTAAGACAC |
| | | GACTTATCGCCACTGGCAGCAGCCACTGGT |
| | | AACAGGATTAGCAGAGCGAGGTATGTAGGC |
| | | GGTGCTACAGAGTTCTTGAAGTGGTGGCCT |
| | | AACTACGGCTACACTAGAAGAACAGTATTT |
| | | GGTATCTGCGCTCTGCTGAAGCCAGTTACC |
| | | TTCGGAAAAAGAGTTGGTAGCTCTTGATCC |
| | | GGCAAACAAACCACCGCTGGTAGCGGTGGT |
| | | TTTTTTGTTTGCAAGCAGCAGATTACGCGC |
| | | AGAAAAAAAGGATCTCAAGAAGATCCTTTG |
| | | ATCTTTTCTACGGGGTCTGACGCTCAGTGG |
| | | AACGAAAACTCACGTTAAGGGATTTTGGTC |
| | | ATGAGATTATCAAAAAGGATCTTCACCTAG |
| | | ATCCTTTTAAATTAAAAATGAAGTTTTAAA |
| | | TCAATCTAAAGTATATATGAGTAAACTTGG |
| | | TCTGACAGTTACCAATGCTTAATCAGTGAG |
| | | GCACCTATCTCAGCGATCTGTCTATTTCGT |
| | | TCATCCATAGTTGCCTGACTCCCCGTCGTG |
| | | TAGATAACTACGATACGGGAGGGCTTACCA |
| | | TCTGGCCCCAGTGCTGCAATGATACCGCGA |
| | | GACCCACGCTCACCGGCTCCAGATTTATCA |
| | | GCAATAAACCAGCCAGCCGGAAGGGCCGAG |
| | | CGCAGAAGTGGTCCTGCAACTTTATCCGCC |
| | | TCCATCCAGTCTATTAATTGTTGCCGGGAA |
| | | GCTAGAGTAAGTAGTTCGCCAGTTAATAGT |
| | | TTGCGCAACGTTGTTGCCATTGCTACAGGC |
| | | ATCGTGGTGTCACGCTCGTCGTTTGGTATG |
| | | GCTTCATTCAGCTCCGGTTCCCAACGATCA |
| | | AGGCGAGTTACATGATCCCCCATGTTGTGC |
| | | AAAAAAGCGGTTAGCTCCTTCGGTCCTCCG |
| | | ATCGTTGTCAGAAGTAAGTTGGCCGCAGTG |
| | | TTATCACTCATGGTTATGGCAGCACTGCAT |
| | | AATTCTCTTACTGTCATGCCATCCGTAAGA |
| | | TGCTTTTCTGTGACTGGTGAGTACTCAACC |
| | | AAGTCATTCTGAGAATAGTGTATGCGGCGA |
| | | CCGAGTTGCTCTTGCCCGGCGTCAATACGG |
| | | GATAATACCGCGCCACATAGCAGAACTTTA |
| | | AAAGTGCTCATCATTGGAAAACGTTCTTCG |
| | | GGGCGAAAACTCTCAAGGATCTTACCGCTG |
| | | TTGAGATCCAGTTCGATGTAACCCACTCGT |
| | | GCACCCAACTGATCTTCAGCATCTTTTACT |
| | | TTCACCAGCGTTTCTGGGTGAGCAAAAACA |
| | | GGAAGGCAAAATGCCGCAAAAAAGGGAATA |
| | | AGGGCGACACGGAAATGTTGAATACTCATA |
| | | CTCTTCCTTTTTCAATATTATTGAAGCATT |
| | | TATCAGGGTTATTGTCTCATGAGCGGATAC |
| | | ATATTTGAATGTATTTAGAAAAATAAACAA |
| | | ATAGGGGTTCCGCGCACATTTCCCCGAAAA |
| | | GTGCCACCTGAC |
| 11 | pPG36 | CTCTGCCCAGCCTGCCACCTTCCTGGCCCG |
| | | TAGCCCTCACGTGGGTGTGAAGGACGTGGA |
| | | GTGTGGGGAAGGACACTTCTGCCATGATAA |
| | | CCAGACCTGCTGCCGAGACAACCGACAGGG |
| | | CTGGGCCTGCTGTCCCTACCGCCAGGGCGT |
| | | CTGTTGTGCTGATCGGCGCCACTGCTGTCC |
| | | TGCTGGCTTCCGCTGCGCAGCCAGGGGTAC |
| | | CAAGTGTTTGCGCAGGGAGGCCCCGCGCTG |
| | | GGACGCCCCTTTGAGGGACCCAGCCTTGAG |
| | | ACAGCTGCTGTGAGGCCAggcCGGCCGAAT |
| | | TCGATATCAAGCTTATCGATAATCAACCTC |
| | | TGGATTACAAAATTTGTGAAAGATTGACTG |
| | | GTATTCTTAACTATGTTGCTCCTTTTACGC |
| | | TATGTGGATACGCTGCTTTAATGCCTTTGT |
| | | ATCATGCTATTGCTTCCCGTATGGCTTTCA |
| | | TTTTCTCCTCCTTGTATAAATCCTGGTTGC |
| | | TGTCTCTTTATGAGGAGTTGTGGCCCGTTG |
| | | TCAGGCAACGTGGCGTGGTGTGCACTGTGT |
| | | TTGCTGACGCAACCCCCACTGGTTGGGGCA |
| | | TTGCCACCACCTGTCAGCTCCTTTCCGGGA |
| | | CTTTCGCTTTCCCCCTCCCTATTGCCACGG |
| | | CGGAACTCATCGCCGCCTGCCTTGCCCGCT |
| | | GCTGGACAGGGGCTCGGCTGTTGGGCACTG |
| | | ACAATTCCGTGGTGTTGTCGGGGAAATCAT |
| | | CGTCCTTTCCTTGGCTGCTCGCCTGTGTTG |
| | | CCACCTGGATTCTGCGCGGGACGTCCTTCT |
| | | GCTACGTCCCTTCGGCCCTCAATCCAGCGG |
| | | ACCTTCCTTCCCGCGGCCTGCTGCCGGCTC |
| | | TGCGGCCTCTTCCGCGTCTTCGCCTTCGCC |
| | | CTCAGACGAGTCGGATCTCCCTTTGGGCCG |
| | | CCTCCCCGCATCGATACCGTCGACCTCGAG |
| | | ACCTAGAAAAACATGGAGCAATCACAAGTA |
| | | GCAATACAGCAGCTACCAATGCTGATTGTG |
| | | CCTGGCTAGAAGCACAAGAGGAGGAGGAGG |
| | | TGGGTTTTCCAGTCACACCTCAGGTACCTT |
| | | TAAGACCAATGACTTACAAGGCAGCTGTAG |
| | | ATCTTAGCCACTTTTTAAAAGAAAAGGGGG |
| | | GACTGGAAGGGCTAATTCACTCCCAACGAA |
| | | GACAAGATATCCTTGATCTGTGGATCTACC |
| | | ACACACAAGGCTACTTCCCTGATTGGCAGA |
| | | ACTACACACCAGGGCCAGGGATCAGATATC |
| | | CACTGACCTTTGGATGGTGCTACAAGCTAG |
| | | TACCAGTTGAGCAAGAGAAGGTAGAAGAAG |
| | | CCAATGAAGGAGAGAACACCCGCTTGTTAC |
| | | ACCCTGTGAGCCTGCATGGGATGGATGACC |
| | | CGGAGAGAGAAGTATTAGAGTGGAGGTTTG |
| | | ACAGCCGCCTAGCATTTCATCACATGGCCC |
| | | GAGAGCTGCATCCGGACTGTACTGGGTCTC |
| | | TCTGGTTAGACCAGATCTGAGCCTGGGAGC |
| | | TCTCTGGCTAACTAGGGAACCCACTGCTTA |
| | | AGCCTCAATAAAGCTTGCCTTGAGTGCTTC |
| | | AAGTAGTGTGTGCCCGTCTGTTGTGTGACT |
| | | CTGGTAACTAGAGATCCCTCAGACCCTTTT |
| | | AGTCAGTGTGGAAAATCTCTAGCAGGGCCC |
| | | GTTTAAACCCGCTGATCAGCCTCGACTGTG |
| | | CCTTCTAGTTGCCAGCCATCTGTTGTTTGC |
| | | CCCTCCCCCGTGCCTTCCTTGACCCTGGAA |
| | | GGTGCCACTCCCACTGTCCTTTCCTAATAA |
| | | AATGAGGAAATTGCATCGCATTGTCTGAGT |
| | | AGGTGTCATTCTATTCTGGGGGGTGGGGTG |
| | | GGGCAGGACAGCAAGGGGGAGGATTGGGAA |
| | | GACAATAGCAGGCATGCTGGGGATGCGGTG |
| | | GGCTCTATGGCTTCTGAGGCGGAAAGAACC |
| | | AGCTGGGGCTCTAGGGGGTATCCCCACGCG |
| | | CCCTGTAGCGGCGCATTAAGCGCGGCGGGT |
| | | GTGGTGGTTACGCGCAGCGTGACCGCTACA |
| | | CTTGCCAGCGCCCTAGCGCCCGCTCCTTTC |
| | | GCTTTCTTCCCTTCCTTTCTCGCCACGTTC |
| | | GCCGGCTTTCCCCGTCAAGCTCTAAATCGG |
| | | GGGCTCCCTTTAGGGTTCCGATTTAGTGCT |
| | | TTACGGCACCTCGACCCCAAAAAACTTGAT |
| | | TAGGGTGATGGTTCACGTAGTGGGCCATCG |
| | | CCCTGATAGACGGTTTTTCGCCCTTTGACG |
| | | TTGGAGTCCACGTTCTTTAATAGTGGACTC |
| | | TTGTTCCAAACTGGAACAACACTCAACCCT |
| | | ATCTCGGTCTATTCTTTTGATTTATAAGGG |
| | | ATTTTGCCGATTTCGGCCTATTGGTTAAAA |
| | | AATGAGCTGATTTAACAAAAATTTAACGCG |
| | | AATTAATTCTGTGGAATGTGTGTCAGTTAG |
| | | GGTGTGGAAAGTCCCCAGGCTCCCCAGCAG |
| | | GCAGAAGTATGCAAAGCATGCATCTCAATT |
| | | AGTCAGCAACCAGGTGTGGAAAGTCCCCAG |
| | | GCTCCCCAGCAGGCAGAAGTATGCAAAGCA |
| | | TGCATCTCAATTAGTCAGCAACCATAGTCC |
| | | CGCCCCTAACTCCGCCCATCCCGCCCCTAA |
| | | CTCCGCCCAGTTCCGCCCATTCTCCGCCCC |
| | | ATGGCTGACTAATTTTTTTTATTTATGCAG |
| | | AGGCCGAGGCCGCCTCTGCCTCTGAGCTAT |
| | | TCCAGAAGTAGTGAGGAGGCTTTTTTGGAG |
| | | GCCTAGGCTTTTGCAAAAAGCTCCCGGGAG |
| | | CTTGTATATCCATTTTCGGATCTGATCAGC |
| | | ACGTGTTGACAATTAATCATCGGCATAGTA |
| | | TATCGGCATAGTATAATACGACAAGGTGAG |
| | | GAACTAAACCATGGCCAAGTTGACCAGTGC |
| | | CGTTCCGGTGCTCACCGCGCGCGACGTCGC |
| | | CGGAGCGGTCGAGTTCTGGACCGACCGGCT |
| | | CGGGTTCTCCCGGGACTTCGTGGAGGACGA |
| | | CTTCGCCGGTGTGGTCCGGGACGACGTGAC |
| | | CCTGTTCATCAGCGCGGTCCAGGACCAGGT |
| | | GGTGCCGGACAACACCCTGGCCTGGGTGTG |
| | | GGTGCGCGGCCTGGACGAGCTGTACGCCGA |
| | | GTGGTCGGAGGTCGTGTCCACGAACTTCCG |
| | | GGACGCCTCCGGGCCGGCCATGACCGAGAT |
| | | CGGCGAGCAGCCGTGGGGGCGGGAGTTCGC |
| | | CCTGCGCGACCCGGCCGGCAACTGCGTGCA |
| | | CTTCGTGGCCGAGGAGCAGGACTGACACGT |
| | | GCTACGAGATTTCGATTCCACCGCCGCCTT |
| | | CTATGAAAGGTTGGGCTTCGGAATCGTTTT |
| | | CCGGGACGCCGGCTGGATGATCCTCCAGCG |
| | | CGGGGATCTCATGCTGGAGTTCTTCGCCCA |
| | | CCCCAACTTGTTTATTGCAGCTTATAATGG |
| | | TTACAAATAAAGCAATAGCATCACAAATTT |
| | | CACAAATAAAGCATTTTTTTCACTGCATTC |
| | | TAGTTGTGGTTTGTCCAAACTCATCAATGT |
| | | ATCTTATCATGTCTGTATACCGTCGACCTC |
| | | TAGCTAGAGCTTGGCGTAATCATGGTCATA |
| | | GCTGTTTCCTGTGTGAAATTGTTATCCGCT |
| | | CACAATTCCACACAACATACGAGCCGGAAG |
| | | CATAAAGTGTAAAGCCTGGGGTGCCTAATG |
| | | AGTGAGCTAACTCACATTAATTGCGTTGCG |
| | | CTCACTGCCCGCTTTCCAGTCGGGAAACCT |
| | | GTCGTGCCAGCTGCATTAATGAATCGGCCA |
| | | ACGCGCGGGGAGAGGCGGTTTGCGTATTGG |
| | | GCGCTCTTCCGCTTCCTCGCTCACTGACTC |
| | | GCTGCGCTCGGTCGTTCGGCTGCGGCGAGC |
| | | GGTATCAGCTCACTCAAAGGCGGTAATACG |
| | | GTTATCCACAGAATCAGGGGATAACGCAGG |
| | | AAAGAACATGTGAGCAAAAGGCCAGCAAAA |
| | | GGCCAGGAACCGTAAAAAGGCCGCGTTGCT |
| | | GGCGTTTTTCCATAGGCTCCGCCCCCCTGA |
| | | CGAGCATCACAAAAATCGACGCTCAAGTCA |
| | | GAGGTGGCGAAACCCGACAGGACTATAAAG |
| | | ATACCAGGCGTTTCCCCCTGGAAGCTCCCT |
| | | CGTGCGCTCTCCTGTTCCGACCCTGCCGCT |
| | | TACCGGATACCTGTCCGCCTTTCTCCCTTC |
| | | GGGAAGCGTGGCGCTTTCTCATAGCTCACG |
| | | CTGTAGGTATCTCAGTTCGGTGTAGGTCGT |
| | | TCGCTCCAAGCTGGGCTGTGTGCACGAACC |
| | | CCCCGTTCAGCCCGACCGCTGCGCCTTATC |
| | | CGGTAACTATCGTCTTGAGTCCAACCCGGT |
| | | AAGACACGACTTATCGCCACTGGCAGCAGC |
| | | CACTGGTAACAGGATTAGCAGAGCGAGGTA |
| | | TGTAGGCGGTGCTACAGAGTTCTTGAAGTG |
| | | GTGGCCTAACTACGGCTACACTAGAAGAAC |
| | | AGTATTTGGTATCTGCGCTCTGCTGAAGCC |
| | | AGTTACCTTCGGAAAAAGAGTTGGTAGCTC |
| | | TTGATCCGGCAAACAAACCACCGCTGGTAG |
| | | CGGTGGTTTTTTTGTTTGCAAGCAGCAGAT |
| | | TACGCGCAGAAAAAAAGGATCTCAAGAAGA |
| | | TCCTTTGATCTTTTCTACGGGGTCTGACGC |
| | | TCAGTGGAACGAAAACTCACGTTAAGGGAT |
| | | TTTGGTCATGAGATTATCAAAAAGGATCTT |
| | | CACCTAGATCCTTTTAAATTAAAAATGAAG |
| | | TTTTAAATCAATCTAAAGTATATATGAGTA |
| | | AACTTGGTCTGACAGTTACCAATGCTTAAT |
| | | CAGTGAGGCACCTATCTCAGCGATCTGTCT |
| | | ATTTCGTTCATCCATAGTTGCCTGACTCCC |
| | | CGTCGTGTAGATAACTACGATACGGGAGGG |
| | | CTTACCATCTGGCCCCAGTGCTGCAATGAT |
| | | ACCGCGAGACCCACGCTCACCGGCTCCAGA |
| | | TTTATCAGCAATAAACCAGCCAGCCGGAAG |
| | | GGCCGAGCGCAGAAGTGGTCCTGCAACTTT |
| | | ATCCGCCTCCATCCAGTCTATTAATTGTTG |
| | | CCGGGAAGCTAGAGTAAGTAGTTCGCCAGT |
| | | TAATAGTTTGCGCAACGTTGTTGCCATTGC |
| | | TACAGGCATCGTGGTGTCACGCTCGTCGTT |
| | | TGGTATGGCTTCATTCAGCTCCGGTTCCCA |
| | | ACGATCAAGGCGAGTTACATGATCCCCCAT |
| | | GTTGTGCAAAAAAGCGGTTAGCTCCTTCGG |
| | | TCCTCCGATCGTTGTCAGAAGTAAGTTGGC |
| | | CGCAGTGTTATCACTCATGGTTATGGCAGC |
| | | ACTGCATAATTCTCTTACTGTCATGCCATC |
| | | CGTAAGATGCTTTTCTGTGACTGGTGAGTA |
| | | CTCAACCAAGTCATTCTGAGAATAGTGTAT |
| | | GCGGCGACCGAGTTGCTCTTGCCCGGCGTC |
| | | AATACGGGATAATACCGCGCCACATAGCAG |
| | | AACTTTAAAAGTGCTCATCATTGGAAAACG |
| | | TTCTTCGGGGCGAAAACTCTCAAGGATCTT |
| | | ACCGCTGTTGAGATCCAGTTCGATGTAACC |
| | | CACTCGTGCACCCAACTGATCTTCAGCATC |
| | | TTTTACTTTCACCAGCGTTTCTGGGTGAGC |
| | | AAAAACAGGAAGGCAAAATGCCGCAAAAAA |
| | | GGGAATAAGGGCGACACGGAAATGTTGAAT |
| | | ACTCATACTCTTCCTTTTTCAATATTATTG |
| | | AAGCATTTATCAGGGTTATTGTCTCATGAG |
| | | CGGATACATATTTGAATGTATTTAGAAAAA |
| | | TAAACAAATAGGGGTTCCGCGCACATTTCC |
| | | CCGAAAAGTGCCACCTGAC |
| 12 | Human PGRN | ATGTGGACCCTGGTGAGCTGGGTGGCCTTA |
| | coding | ACAGCAGGGCTGGTGGCTGGAACGCGGTGC |
| | sequence | CCAGATGGTCAGTTCTGCCCTGTGGCCTGC |
| | | TGCCTGGACCCCGGAGGAGCCAGCTACAGC |
| | | TGCTGCCGTCCCCTTCTGGACAAATGGCCC |
| | | ACAACACTGAGCAGGCATCTGGGTGGCCCC |
| | | TGCCAGGTTGATGCCCACTGCTCTGCCGGC |
| | | CACTCCTGCATCTTTACCGTCTCAGGGACT |
| | | TCCAGTTGCTGCCCCTTCCCAGAGGCCGTG |
| | | GCATGCGGGGATGGCCATCACTGCTGCCCA |
| | | CGGGGCTTCCACTGCAGTGCAGACGGGCGA |
| | | TCCTGCTTCCAAAGATCAGGTAACAACTCC |
| | | GTGGGTGCCATCCAGTGCCCTGATAGTCAG |
| | | TTCGAATGCCCGGACTTCTCCACGTGCTGT |
| | | GTTATGGTCGATGGCTCCTGGGGGTGCTGC |
| | | CCCATGCCCCAGGCTTCCTGCTGTGAAGAC |
| | | AGGGTGCACTGCTGTCCGCACGGTGCCTTC |
| | | TGCGACCTGGTTCACACCCGCTGCATCACA |
| | | CCCACGGGCACCCACCCCCTGGCAAAGAAG |
| | | CTCCCTGCCCAGAGGACTAACAGGGCAGTG |
| | | GCCTTGTCCAGCTCGGTCATGTGTCCGGAC |
| | | GCACGGTCCCGGTGCCCTGATGGTTCTACC |
| | | TGCTGTGAGCTGCCCAGTGGGAAGTATGGC |
| | | TGCTGCCCAATGCCCAACGCCACCTGCTGC |
| | | TCCGATCACCTGCACTGCTGCCCCCAAGAC |
| | | ACTGTGTGTGACCTGATCCAGAGTAAGTGC |
| | | CTCTCCAAGGAGAACGCTACCACGGACCTC |
| | | CTCACTAAGCTGCCTGCGCACACAGTGGGG |
| | | GATGIGAAATGTGACATGGAGGTGAGCTGC |
| | | CCAGATGGCTATACCTGCTGCCGTCTACAG |
| | | TCGGGGGCCTGGGGCTGCTGCCCTTTTACC |
| | | CAGGCTGTGTGCTGTGAGGACCACATACAC |
| | | TGCTGTCCCGCGGGGTTTACGTGTGACACG |
| | | CAGAAGGGTACCTGTGAACAGGGGCCCCAC |
| | | CAGGTGCCCTGGATGGAGAAGGCCCCAGCT |
| | | CACCTCAGCCTGCCAGACCCACAAGCCTTG |
| | | AAGAGAGATGTCCCCTGTGATAATGTCAGC |
| | | AGCTGTCCCTCCTCCGATACCTGCTGCCAA |
| | | CTCACGTCTGGGGAGTGGGGCTGCTGTCCA |
| | | ATCCCAGAGGCTGTCTGCTGCTCGGACCAC |
| | | CAGCACTGCTGCCCCCAGGGCTACACGTGT |
| | | GTAGCTGAGGGGCAGTGTCAGCGAGGAAGC |
| | | GAGATCGTGGCTGGACTGGAGAAGATGCCT |
| | | GCCCGCCGGGCTTCCTTATCCCACCCCAGA |
| | | GACATCGGCTGTGACCAGCACACCAGCTGC |
| | | CCGGTGGGGCAGACCTGCTGCCCGAGCCTG |
| | | GGTGGGAGCTGGGCCTGCTGCCAGTTGCCC |
| | | CATGCTGTGTGCTGCGAGGATCGCCAGCAC |
| | | TGCTGCCCGGCTGGCTACACCTGCAACGTG |
| | | AAGGCTCGATCCTGCGAGAAGGAAGTGGTC |
| | | TCTGCCCAGCCTGCCACCTTCCTGGCCCGT |
| | | AGCCCTCACGTGGGTGTGAAGGACGTGGAG |
| | | TGTGGGGAAGGACACTTCTGCCATGATAAC |
| | | CAGACCTGCTGCCGAGACAACCGACAGGGC |
| | | TGGGCCTGCTGTCCCTACCGCCAGGGCGTC |
| | | TGTTGTGCTGATCGGCGCCACTGCTGTCCT |
| | | GCTGGCTTCCGCTGCGCAGCCAGGGGTACC |
| | | AAGTGTTTGCGCAGGGAGGCCCCGCGCTGG |
| | | GACGCCCCTTTGAGGGACCCAGCCTTGAGA |
| | | CAGCTGCTGTGA |
| 13 | Human PGRN | MWTLVSWVALTAGLVAGTRCPDGQFCPVAC |
| | amino acid | CLDPGGASYSCCRPLLDKWPTTLSRHLGGP |
| | sequence | CQVDAHCSAGHSCIFTVSGTSSCCPFPEAV |
| | | ACGDGHHCCPRGFHCSADGRSCFQRSGNNS |
| | | VGAIQCPDSQFECPDFSTCCVMVDGSWGCC |
| | | PMPQASCCEDRVHCCPHGAFCDLVHTRCIT |
| | | PTGTHPLAKKLPAQRTNRAVALSSSVMCPD |
| | | ARSRCPDGSTCCELPSGKYGCCPMPNATCC |
| | | SDHLHCCPQDTVCDLIQSKCLSKENATTDL |
| | | LTKLPAHTVGDVKCDMEVSCPDGYTCCRLQ |
| | | SGAWGCCPFTQAVCCEDHIHCCPAGFTCDT |
| | | QKGTCEQGPHQVPWMEKAPAHLSLPDPQAL |
| | | KRDVPCDNVSSCPSSDTCCQLTSGEWGCCP |
| | | IPEAVCCSDHQHCCPQGYTCVAEGQCQRGS |
| | | EIVAGLEKMPARRASLSHPRDIGCDQHTSC |
| | | PVGQTCCPSLGGSWACCQLPHAVCCEDROH |
| | | CCPAGYTCNVKARSCEKEVVSAQPATFLAR |
| | | SPHVGVKDVECGEGHFCHDNQTCCRDNRQG |
| | | WACCPYRQGVCCADRRHCCPAGFRCAARGT |
| | | KCLRREAPRWDAPLRDPALROLL |
| 14 | Age1 | ACCGGT |
| | restriction | |
| | site (5′to | |
| | 3′ | |
| | direction) | |
| 15 | WPRE | TCAACCTCTGGATTACAAAATTTGTGAAAG |
| | | ATTGACTGGTATTCTTAACTATGTTGCTCC |
| | | TTTTACGCTATGTGGATACGCTGCTTTAAT |
| | | GCCTTTGTATCATGCTATTGCTTCCCGTAT |
| | | GGCTTTCATTTTCTCCTCCTTGTATAAATC |
| | | CTGGTTGCTGTCTCTTTATGAGGAGTTGTG |
| | | GCCCGTTGTCAGGCAACGTGGCGTGGTGTG |
| | | CACTGTGTTTGCTGACGCAACCCCCACTGG |
| | | TTGGGGCATTGCCACCACCTGTCAGCTCCT |
| | | TTCCGGGACTTTCGCTTTCCCCCTCCCTAT |
| | | TGCCACGGCGGAACTCATCGCCGCCTGCCT |
| | | TGCCCGCTGCTGGACAGGGGCTCGGCTGTT |
| | | GGGCACTGACAATTCCGTGGTGTTGTCGGG |
| | | GAAATCATCGTCCTTTCCTTGGCTGCTCGC |
| | | CTGTGTTGCCACCTGGATTCTGCGCGGGAC |
| | | GTCCTTCTGCTACGTCCCTTCGGCCCTCAA |
| | | TCCAGCGGACCTTCCTTCCCGCGGCCTGCT |
| | | GCCGGCTCTGCGGCCTCTTCCGCGTCTTCG |
| | | CCTTCGCCCTCAGACGAGTCGGATCTCCCT |
| | | TTGGGCCGCCTCCCCGCA |
| 16 | PolyA signal | gatccagacatgataagatacattgatgag |
| | sequence | tttggacaaaccacaactagaatgcagtga |
| | (SV40) | aaaaaatgctttatttgtgaaatttgtgat |
| | | gctattgctttatttgtaaccattataagc |
| | | tgcaataaacaagttaacaacaacaattgc |
| | | attcattttatgtttcaggttcagggggag |
| | | gtgtgggaggttttttag |
| 17 | AAVTT-pPG36 | GCGCGCTCGCTCGCTCACTGAGGCCGCCCG |
| | | GGCAAAGCCCGGGCGTCGGGCGACCTTTGG |
| | | TCGCCCGGCCTCAGTGAGCGAGCGAGCGCG |
| | | CAGAGAGGGAGTGGCCAACTCCATCACTAG |
| | | GGGTTCCTTGTAGTTAATGATTAACCTCTG |
| | | ctagcAGCTGAATGGGGTCCGCCTCTTTTC |
| | | CCTGCCTAAACAGACAGGAACTCCTGCCAA |
| | | TTGAGGGCGTCACCGCTAAGGCTCCGCCCC |
| | | AGCCTGGGCTCCACAACCAATGAAGGGTAA |
| | | TCTCGACAAAGAGCAAGGGGTGGGGCGCGG |
| | | GCGCGCAGGTGCAGCAGCACACAGGCTGGT |
| | | CGGGAGGGCGGGGCGCGACGTCTGCCGTGC |
| | | GGGGTCCCGGCATCGGTTGCGCGCACCGGT |
| | | gcgctccctcctctcggagagagggctgtg |
| | | gtaaaacccgtccggaaaTtggccgccgct |
| | | gccgccaccgccgccgccgccgccgcgccg |
| | | agcggaggaggaggaggaggcgaggaggag |
| | | agactgtgagtgggaccgccaaggccgcgg |
| | | ggcgggacccttgctggggggcgggtaggg |
| | | gcgggacgtggcgcgggaggggcccgcggg |
| | | gtcgggcgacacggctggcggTtggcgtcc |
| | | ctcctctctaccctccccctccctctgccg |
| | | ccggtggtggctttctccactcgtctcccg |
| | | caatcgcgagcgacggttctcagcgcgatc |
| | | tccctggagccaccttcgaTtgacgccctc |
| | | ccgctgcccgccccatctgtgcgcatccta |
| | | ggccccagctgtgcaagcgcccttgtcgtc |
| | | tgggcttcgccagttggggctgcgcgcgct |
| | | cctgcccttcttggggctttgggcctcggc |
| | | actgtcgcgcgcccgcggtcccggcctctc |
| | | cctggatcgcgctgtccccttctccctcgc |
| | | gcgcccccactcccgttacttgctcccccc |
| | | tcacacacacagactggcgcgcgtgcgcag |
| | | tccatctcccgttgggagagtgcgccacaa |
| | | gggctcctgagctcttacccccatctctgg |
| | | gttttgctccctcctcctcctctcccattc |
| | | cgtgactttttgcccccactgcaagcgagt |
| | | cggtccatcagctccattccccacttggca |
| | | ggaacaagttgagggttattgtccacccac |
| | | aaaaaggactagacattTtgttcctaggtc |
| | | ccacaactcatcataaagagTtggttgtag |
| | | ttctcatcaggaaccgtgggcaagggactg |
| | | tgcgttcctcagcactcgaagctcttccgt |
| | | gagaccTtgcccgcagggtgctctggttct |
| | | ttggggTtgctgtgctgtggcttcggaatT |
| | | tgagcgtcttcccaccctccctcccctccc |
| | | ttcgccagcgttctgtctacaagaaagaat |
| | | aggcaggtgtccttggatatCgtagttgct |
| | | aatCgcctatacactgttctattacacctt |
| | | tctgctaaggatagggtttttggttttggt |
| | | tttggttttgttccccaccctccagtttgg |
| | | tttagttttggttttggcatccaagatacc |
| | | ttttttctcatactggaaccctaggcagca |
| | | gttgctatttccctgagttagcaatagttt |
| | | tacagtattttgaggccttttgtccataat |
| | | tctcacggaatCcctcagggatCagattag |
| | | ctgctgttgggatCaggaaattgggttaca |
| | | ccgctgaaatctcttgctggggcccttgtt |
| | | ttgaattggaaagtcaggaggctggaacga |
| | | aggctcacaagttaacagtgccagctgctc |
| | | ttccagaagccctggattcagtcccaccaa |
| | | tccatCgcgggtcacaaccatctgtaactt |
| | | cagtcccaaggggtccgaAgccctcttctg |
| | | gctttgccctattattttatttatcttatC |
| | | tgtttttgtcttgtcatCtggcaagcccag |
| | | ggggccattgggtgcaacttataaactgac |
| | | ttctgtatCttaagaagccaaccatacagt |
| | | gcttacattccagaaaaaaaatCtgccact |
| | | ttaacagcactagaactagggtttagagaa |
| | | gtatCataaaggtcaaatatCtttgaccaa |
| | | tatcaccagcaacctaaagctgttaagaaa |
| | | tctttgggccccagcttgacccaaggatac |
| | | agtatcctagggaagttaccaaaatcagag |
| | | atagtatgcagcagccaggggtctcatgtg |
| | | tggcactcaagctcacctatactcactact |
| | | gtgcagacagctgtgttctctgtaatactt |
| | | acatatttgtttaatacttcagggaggaaa |
| | | agtcagaagaccaggatctccagggcctca |
| | | ACCGGTGGCCCaggCGGCCACCATGTGGAC |
| | | CCTGGTGAGCTGGGTGGCCTTAACAGCAGG |
| | | GCTGGTGGCTGGAACGCGGTGCCCAGATGG |
| | | TCAGTTCTGCCCTGTGGCCTGCTGCCTGGA |
| | | CCCCGGAGGAGCCAGCTACAGCTGCTGCCG |
| | | TCCCCTTCTGGACAAATGGCCCACAACACT |
| | | GAGCAGGCATCTGGGTGGCCCCTGCCAGGT |
| | | TGATGCCCACTGCTCTGCCGGCCACTCCTG |
| | | CATCTTTACCGTCTCAGGGACTTCCAGTTG |
| | | CTGCCCCTTCCCAGAGGCCGTGGCATGCGG |
| | | GGATGGCCATCACTGCTGCCCACGGGGCTT |
| | | CCACTGCAGTGCAGACGGGCGATCCTGCTT |
| | | CCAAAGATCAGGTAACAACTCCGTGGGTGC |
| | | CATCCAGTGCCCTGATAGTCAGTTCGAATG |
| | | CCCGGACTTCTCCACGTGCTGTGTTATGGT |
| | | CGATGGCTCCTGGGGGTGCTGCCCCATGCC |
| | | CCAGGCTTCCTGCTGTGAAGACAGGGTGCA |
| | | CTGCTGTCCGCACGGTGCCTTCTGCGACCT |
| | | GGTTCACACCCGCTGCATCACACCCACGGG |
| | | CACCCACCCCCTGGCAAAGAAGCTCCCTGC |
| | | CCAGAGGACTAACAGGGCAGTGGCCTTGTC |
| | | CAGCTCGGTCATGTGTCCGGACGCACGGTC |
| | | CCGGTGCCCTGATGGTTCTACCTGCTGTGA |
| | | GCTGCCCAGTGGGAAGTATGGCTGCTGCCC |
| | | AATGCCCAACGCCACCTGCTGCTCCGATCA |
| | | CCTGCACTGCTGCCCCCAAGACACTGTGTG |
| | | TGACCTGATCCAGAGTAAGTGCCTCTCCAA |
| | | GGAGAACGCTACCACGGACCTCCTCACTAA |
| | | GCTGCCTGCGCACACAGTGGGGGATGTGAA |
| | | ATGTGACATGGAGGTGAGCTGCCCAGATGG |
| | | CTATACCTGCTGCCGTCTACAGTCGGGGGC |
| | | CTGGGGCTGCTGCCCTTTTACCCAGGCTGT |
| | | GTGCTGTGAGGACCACATACACTGCTGTCC |
| | | CGCGGGGTTTACGTGTGACACGCAGAAGGG |
| | | TACCTGTGAACAGGGGCCCCACCAGGTGCC |
| | | CTGGATGGAGAAGGCCCCAGCTCACCTCAG |
| | | CCTGCCAGACCCACAAGCCTTGAAGAGAGA |
| | | TGTCCCCTGTGATAATGTCAGCAGCTGTCC |
| | | CTCCTCCGATACCTGCTGCCAACTCACGTC |
| | | TGGGGAGTGGGGCTGCTGTCCAATCCCAGA |
| | | GGCTGTCTGCTGCTCGGACCACCAGCACTG |
| | | CTGCCCCCAGGGCTACACGTGTGTAGCTGA |
| | | GGGGCAGTGTCAGCGAGGAAGCGAGATCGT |
| | | GGCTGGACTGGAGAAGATGCCTGCCCGCCG |
| | | GGCTTCCTTATCCCACCCCAGAGACATCGG |
| | | CTGTGACCAGCACACCAGCTGCCCGGTGGG |
| | | GCAGACCTGCTGCCCGAGCCTGGGTGGGAG |
| | | CTGGGCCTGCTGCCAGTTGCCCCATGCTGT |
| | | GTGCTGCGAGGATCGCCAGCACTGCTGCCC |
| | | GGCTGGCTACACCTGCAACGTGAAGGCTCG |
| | | ATCCTGCGAGAAGGAAGTGGTCTCTGCCCA |
| | | GCCTGCCACCTTCCTGGCCCGTAGCCCTCA |
| | | CGTGGGTGTGAAGGACGTGGAGTGTGGGGA |
| | | AGGACACTTCTGCCATGATAACCAGACCTG |
| | | CTGCCGAGACAACCGACAGGGCTGGGCCTG |
| | | CTGTCCCTACCGCCAGGGCGTCTGTTGTGC |
| | | TGATCGGCGCCACTGCTGTCCTGCTGGCTT |
| | | CCGCTGCGCAGCCAGGGGTACCAAGTGTTT |
| | | GCGCAGGGAGGCCCCGCGCTGGGACGCCCC |
| | | TTTGAGGGACCCAGCCTTGAGACAGCTGCT |
| | | GTGAGGCCAggcCGGCCGaattcGATCCAG |
| | | ACATGATAAGATACATTGATGAGTTTGGAC |
| | | AAACCACAACTAGAATGCAGTGAAAAAAAT |
| | | GCTTTATTTGTGAAATTTGTGATGCTATTG |
| | | CTTTATTTGTAACCATTATAAGCTGCAATA |
| | | AACAAGTTAACAACAACAATTGCATTCATT |
| | | TTATGTTTCAGGTTCAGGGGGAGGTGTGGG |
| | | AGGTTTTTTAGGGATCCTCAGgttaatcat |
| | | taactacaaggaacccctagtgatggagtt |
| | | ggccactccctctctgcgcgctcgctcgct |
| | | cactgaggccgggcgaccaaaggtcgcccg |
| | | acgcccgggctttgcccgggggcgtgagcg |
| | | agcgagcgcgc |
| 18 | AAVTT-p1PG36 | AATAAATTGCAGTTTCATTTGATGCTCGAT |
| | | GAGTTTTTCTAACTCATGACCAAAATCCCT |
| | | TAACGTGAGTTACGCGCGCGTCGTTCCACT |
| | | GAGCGTCAGACCCCGTAGAAAAGATCAAAG |
| | | GATCTTCTTGAGATCCTTTTTTTCTGCGCG |
| | | TAATCTGCTGCTTGCAAACAAAAAAACCAC |
| | | CGCTACCAGCGGTGGTTTGTTTGCCGGATC |
| | | AAGAGCTACCAACTCTTTTTCCGAAGGTAA |
| | | CTGGCTTCAGCAGAGCGCAGATACCAAATA |
| | | CTGTTCTTCTAGTGTAGCCGTAGTTAGCCC |
| | | ACCACTTCAAGAACTCTGTAGCACCGCCTA |
| | | CATACCTCGCTCTGCTAATCCTGTTACCAG |
| | | TGGCTGCTGCCAGTGGCGATAAGTCGTGTC |
| | | TTACCGGGTTGGACTCAAGACGATAGTTAC |
| | | CGGATAAGGCGCAGCGGTCGGGCTGAACGG |
| | | GGGGTTCGTGCACACAGCCCAGCTTGGAGC |
| | | GAACGACCTACACCGAACTGAGATACCTAC |
| | | AGCGTGAGCTATGAGAAAGCGCCACGCTTC |
| | | CCGAAGGGAGAAAGGCGGACAGGTATCCGG |
| | | TAAGCGGCAGGGTCGGAACAGGAGAGCGCA |
| | | CGAGGGAGCTTCCAGGGGGAAACGCCTGGT |
| | | ATCTTTATAGTCCTGTCGGGTTTCGCCACC |
| | | TCTGACTTGAGCGTCGATTTTTGTGATGCT |
| | | CGTCAGGGGGGCGGAGCCTATGGAAAAACG |
| | | CCAGCAACGCGGCCTTTTTACGGTTCCTGG |
| | | CCTTTTGCTGGCCTTTTGCTCACATGTTCT |
| | | TTCCTGCGTTATCCCCTGATTCTGTGGATA |
| | | ACCGTATTACCGCCTTTGAGTGAGCTGATA |
| | | CCGCTCAAGGCTGACTGCAGGGCGAGAAGA |
| | | TTGCGAGCTGTGCGGCTGAGTTGACGTATC |
| | | TGTGCTGGATGATTACTCATAACGGCACCG |
| | | CTATCAAACGTGCCACGTTCATGTCCTACA |
| | | GCGCGCTCGCTCGCTCACTGAGGCCGCCCG |
| | | GGCAAAGCCCGGGCGTCGGGCGACCTTTGG |
| | | TCGCCCGGCCTCAGTGAGCGAGCGAGCGCG |
| | | CAGAGAGGGAGTGGCCAACTCCATCACTAG |
| | | GGGTTCCTTGTAGTTAATGATTAACCTCTG |
| | | ctagcAGCTGAATGGGGTCCGCCTCTTTTC |
| | | CCTGCCTAAACAGACAGGAACTCCTGCCAA |
| | | TTGAGGGCGTCACCGCTAAGGCTCCGCCCC |
| | | AGCCTGGGCTCCACAACCAATGAAGGGTAA |
| | | TCTCGACAAAGAGCAAGGGGTGGGGCGCGG |
| | | GCGCGCAGGTGCAGCAGCACACAGGCTGGT |
| | | CGGGAGGGCGGGGCGCGACGTCTGCCGTGC |
| | | GGGGTCCCGGCATCGGTTGCGCGCACCGGT |
| | | gcgctccctcctctcggagagagggctgtg |
| | | gtaaaacccgtccggaaaTtggccgccgct |
| | | gccgccaccgccgccgccgccgccgcgccg |
| | | agcggaggaggaggaggaggcgaggaggag |
| | | agactgtgagtgggaccgccaaggccgcgg |
| | | ccctccccctccctctgccgccggtggtgg |
| | | ctttctccactcgtctcccgcaatcgcgag |
| | | cgacggttctcagcgcgatctccctggagc |
| | | caccttcgaTtgacgccctcccgctgcccg |
| | | ccccatctgtgcgcatcctaggccccagct |
| | | gtgcaagcgcccttgtcgtctgggcttcgc |
| | | cagttggggctgcgcgcgct |
| | | cctgcccttcttggggctttgggcctcggc |
| | | actgtcgcgcgcccgcggtcccggcctctc |
| | | cctggatcgcgctgtccccttctccctcgc |
| | | gcgcccccactcccgttacttgctcccccc |
| | | tcacacacacagactggcgcgcgtgcgcag |
| | | tccatctcccgttgggagagtgcgccacaa |
| | | gggctcctgagctcttacccccatctctgg |
| | | gttttgctccctcctcctcctctcccattc |
| | | cgtgactttttgcccccactgcaagcgagt |
| | | cggtccatcagctccattccccacttggca |
| | | ggaacaagttgagggttattgtccacccac |
| | | aaaaaggactagacattTtgttcctaggtc |
| | | ccacaactcatcataaagagTtggttgtag |
| | | ttctcatcaggaaccgtgggcaagggactg |
| | | tgcgttcctcagcactcgaagctcttccgt |
| | | gagaccTtgcccgcagggtgctctggttct |
| | | ttggggTtgctgtgctgtggcttcggaatT |
| | | tgagcgtcttcccaccctccctcccctccc |
| | | ttcgccagcgttctgtctacaagaaagaat |
| | | aggcaggtgtccttggatatCgtagttgct |
| | | aatCgcctatacactgttctattacacctt |
| | | tctgctaaggatagggtttttggttttggt |
| | | tttggttttgttccccaccctccagtttgg |
| | | tttagttttgcccatCtgacccaagatacc |
| | | ttttttctcatactggaaccctaggcagca |
| | | gttgctatttccctgagttagcaatagttt |
| | | tacagtattttgaggccttttgtccataat |
| | | tctcacggaatCcctcagggatCagattag |
| | | ctgctgttgggatCaggaaattgggttaca |
| | | ccgctgaaatctcttgctggggcccttgtt |
| | | ttgaattggaaagtcaggaggctggaacga |
| | | aggctcacaagttaacagtgccagctgctc |
| | | ttccagaagccctggattcagtcccaccaa |
| | | tccatCgcgggtcacaaccatctgtaactt |
| | | cagtcccaaggggtccgaAgccctcttctg |
| | | gctttgccctattattttatttatcttatC |
| | | tgtttttgtcttgtcatCtggcaagcccag |
| | | ggggccattgggtgcaacttataaactgac |
| | | ttctgtatCttaagaagccaaccatacagt |
| | | gcttacattccagaaaaaaaatCtgccact |
| | | ttaacagcactagaactagggtttagagaa |
| | | gtatCataaaggtcaaatatCtttgaccaa |
| | | tatcaccagcaacctaaagctgttaagaaa |
| | | tctttgggccccagcttgacccaaggatac |
| | | agtatcctagggaagttaccaaaatcagag |
| | | atagtatgcagcagccaggggtctcatgtg |
| | | tggcactcaagctcacctatactcactact |
| | | gtgcagacagctgtgttctctgtaatactt |
| | | acatatttgtttaatacttcagggaggaaa |
| | | agtcagaagaccaggatctccagggcctca |
| | | ACCGGTGGCCCaggCGGCCACCATGTGGAC |
| | | CCTGGTGAGCTGGGTGGCCTTAACAGCAGG |
| | | GCTGGTGGCTGGAACGCGGTGCCCAGATGG |
| | | TCAGTTCTGCCCTGTGGCCTGCTGCCTGGA |
| | | CCCCGGAGGAGCCAGCTACAGCTGCTGCCG |
| | | TCCCCTTCTGGACAAATGGCCCACAACACT |
| | | GAGCAGGCATCTGGGTGGCCCCTGCCAGGT |
| | | TGATGCCCACTGCTCTGCCGGCCACTCCTG |
| | | CATCTTTACCGTCTCAGGGACTTCCAGTTG |
| | | CTGCCCCTTCCCAGAGGCCGTGGCATGCGG |
| | | GGATGGCCATCACTGCTGCCCACGGGGCTT |
| | | CCACTGCAGTGCAGACGGGCGATCCTGCTT |
| | | CCAAAGATCAGGTAACAACTCCGTGGGTGC |
| | | CATCCAGTGCCCTGATAGTCAGTTCGAATG |
| | | CCCGGACTTCTCCACGTGCTGTGTTATGGT |
| | | CGATGGCTCCTGGGGGTGCTGCCCCATGCC |
| | | CCAGGCTTCCTGCTGTGAAGACAGGGTGCA |
| | | CTGCTGTCCGCACGGTGCCTTCTGCGACCT |
| | | GGTTCACACCCGCTGCATCACACCCACGGG |
| | | CACCCACCCCCTGGCAAAGAAGCTCCCTGC |
| | | CCAGAGGACTAACAGGGCAGTGGCCTTGTC |
| | | CAGCTCGGTCATGTGTCCGGACGCACGGTC |
| | | CCGGTGCCCTGATGGTTCTACCTGCTGTGA |
| | | GCTGCCCAGTGGGAAGTATGGCTGCTGCCC |
| | | AATGCCCAACGCCACCTGCTGCTCCGATCA |
| | | CCTGCACTGCTGCCCCCAAGACACTGTGTG |
| | | TGACCTGATCCAGAGTAAGTGCCTCTCCAA |
| | | GGAGAACGCTACCACGGACCTCCTCACTAA |
| | | GCTGCCTGCGCACACAGTGGGGGATGTGAA |
| | | ATGTGACATGGAGGTGAGCTGCCCAGATGG |
| | | CTATACCTGCTGCCGTCTACAGTCGGGGGC |
| | | CTGGGGCTGCTGCCCTTTTACCCAGGCTGT |
| | | GTGCTGTGAGGACCACATACACTGCTGTCC |
| | | CGCGGGGTTTACGTGTGACACGCAGAAGGG |
| | | TACCTGTGAACAGGGGCCCCACCAGGTGCC |
| | | CTGGATGGAGAAGGCCCCAGCTCACCTCAG |
| | | CCTGCCAGACCCACAAGCCTTGAAGAGAGA |
| | | TGTCCCCTGTGATAATGTCAGCAGCTGTCC |
| | | CTCCTCCGATACCTGCTGCCAACTCACGTC |
| | | TGGGGAGTGGGGCTGCTGTCCAATCCCAGA |
| | | GGCTGTCTGCTGCTCGGACCACCAGCACTG |
| | | CTGCCCCCAGGGCTACACGTGTGTAGCTGA |
| | | GGGGCAGTGTCAGCGAGGAAGCGAGATCGT |
| | | GGCTGGACTGGAGAAGATGCCTGCCCGCCG |
| | | GGCTTCCTTATCCCACCCCAGAGACATCGG |
| | | CTGTGACCAGCACACCAGCTGCCCGGTGGG |
| | | GCAGACCTGCTGCCCGAGCCTGGGTGGGAG |
| | | CTGGGCCTGCTGCCAGTTGCCCCATGCTGT |
| | | GTGCTGCGAGGATCGCCAGCACTGCTGCCC |
| | | GGCTGGCTACACCTGCAACGTGAAGGCTCG |
| | | ATCCTGCGAGAAGGAAGTGGTCTCTGCCCA |
| | | GCCTGCCACCTTCCTGGCCCGTAGCCCTCA |
| | | CGTGGGTGTGAAGGACGTGGAGTGTGGGGA |
| | | AGGACACTTCTGCCATGATAACCAGACCTG |
| | | CTGCCGAGACAACCGACAGGGCTGGGCCTG |
| | | CTGTCCCTACCGCCAGGGCGTCTGTTGTGC |
| | | TGATCGGCGCCACTGCTGTCCTGCTGGCTT |
| | | CCGCTGCGCAGCCAGGGGTACCAAGTGTTT |
| | | GCGCAGGGAGGCCCCGCGCTGGGACGCCCC |
| | | TTTGAGGGACCCAGCCTTGAGACAGCTGCT |
| | | GTGAGGCCAggcCGGCCGaattcGATCCAG |
| | | ACATGATAAGATACATTGATGAGTTTGGAC |
| | | AAACCACAACTAGAATGCAGTGAAAAAAAT |
| | | GCTTTATTTGTGAAATTTGTGATGCTATTG |
| | | CTTTATTTGTAACCATTATAAGCTGCAATA |
| | | AACAAGTTAACAACAACAATTGCATTCATT |
| | | TTATGTTTCAGGTTCAGGGGGAGGTGTGGG |
| | | AGGTTTTTTAGGGATCCTCAGgttaatcat |
| | | taactacaaggaacccctagtgatggagtt |
| | | ggccactccctctctgcgcgctcgctcgct |
| | | cactgaggccgggcgaccaaaggtcgcccg |
| | | acgcccgggctttgcccgggcggcctcagt |
| | | gagcgagcgagcgcgcACTGTCATTAGCAA |
| | | CTCCTTGTCCTTCGATCTCGTCAACAACAG |
| | | CTTGCAGTTCAAATACAAGACCCAGAAGGC |
| | | GACTATTCTGGAAGCGAGCTTGAAGAGTTA |
| | | ACCTGCAGAGAGCCCCCGCAGTGTCGACAA |
| | | TTAATCATCGGCATAGTATATCGGCATAGT |
| | | ATAATACGACAAGGTGAGGAAGTAAAAAAT |
| | | GAGCCATATCCAACGGGAAACGTCGAGGCC |
| | | GCGATTAAATTCCAACATGGATGCTGATTT |
| | | ATATGGGTATAAATGGGCTCGCGATAATGT |
| | | CGGGCAATCAGGTGCGACAATCTATCGCTT |
| | | GTATGGGAAGCCCGATGCGCCAGAGTIGTT |
| | | TCTGAAACATGGCAAAGGTAGCGTTGCCAA |
| | | TGATGTTACAGATGAGATGGTCAGACTAAA |
| | | CTGGCTGACGGAATTTATGCCACTTCCGAC |
| | | CATCAAGCATTTTATCCGTACTCCTGATGA |
| | | TGCATGGTTACTCACCACTGCGATCCCCGG |
| | | AAAAACAGCGTTCCAGGTATTAGAAGAATA |
| | | TCCTGATTCAGGTGAAAATATTGTTGATGC |
| | | GCTGGCAGTGTTCCTGCGCCGGTTGCACTC |
| | | GATTCCTGTTTGTAATTGTCCTTTTAACAG |
| | | CGATCGCGTATTTCGCCTCGCTCAGGCGCA |
| | | ATCACGAATGAATAACGGTTTGGTTGATGC |
| | | GAGTGATTTTGATGACGAGCGTAATGGCTG |
| | | GCCTGTTGAACAAGTCTGGAAAGAAATGCA |
| | | TAAACTTTTGCCATTCTCACCGGATTCAGT |
| | | CGTCACTCATGGTGATTTCTCACTTGATAA |
| | | CCTTATTTTTGACGAGGGGAAATTAATAGG |
| | | TTGTATTGATGTTGGACGAGTCGGAATCGC |
| | | AGACCGATACCAGGATCTTGCCATCCTATG |
| | | GAACTGCCTCGGTGAGTTTTCTCCTTCATT |
| | | ACAGAAACGGCTTTTTCAAAAATATGGTAT |
| | | TGATAATCCTGATATG |
| 19 | AAVTT-p2PG36 | gaagcattttgttaaaattcgcgttaaatt |
| | | tttgttaaatcagctattttttaaccaata |
| | | ggccgaaatcggcaaaatcccttgtaaatc |
| | | aaaagaatagaccgagatagggttgagtgt |
| | | tgttccagtttggaacaagagtccactatt |
| | | aaagaacgtggactccaacgtcaaagggcg |
| | | aaaaaccgtctatcagggcgttggcccact |
| | | acgtgaaccttcaccctaatcaagtttttt |
| | | ggggtcgaggtgccgtaaagcactaaatcg |
| | | gaaccctaaagggagcccccgatttagagc |
| | | ttgacggggaaaccggcgaacgtggcgaga |
| | | aaggaagggaagaaagcgaaaggagcgggc |
| | | gctagggcgctggcaagtgtagcggtcacg |
| | | ctgcgcgtaaccaccacacccgccgcgcta |
| | | agcgccgctacagggcgcgtcccttcgcct |
| | | tcaggctgcgtcgagtactgtactgtgagc |
| | | cagagttgcccggcgctctccggctgcggt |
| | | agttcaggcagttcaatcaactgtttacct |
| | | tgtggagcgactccagaggcacttcaccgc |
| | | ttgccagcggcttacgatccagcgccacga |
| | | tccagtgcaggagatcgttatcgctatacg |
| | | gaacaggtattcgctggtcacttcgataag |
| | | gtttgcccggataaacggaactggaaaaac |
| | | tgctgctggtcgttctaacagaactggcga |
| | | ttgttcggcgtatcgccaaaatcaccgccg |
| | | taagccgaccacgggttgccgttttcagca |
| | | ggatttaatcagcgactgatccacccagtc |
| | | ccagacgaagccgccctgtaaacggggata |
| | | ctgacgaaacgcctgccagtatttagcgaa |
| | | accgccaagactgttacccaagcgtgggcg |
| | | tattcgcaaaggatcagcgggcgcgtctct |
| | | ccaggtagcgaaagccttttttgatcgacc |
| | | tttcggcacagccgggaagggctggtcttc |
| | | aaccacgcgcgcgtacaacgggcaaataat |
| | | atcggtggccgtggtgtcggctccgccgcc |
| | | ttcaactgcaccgggcgggaaggatcgaca |
| | | gatttgatccagcgatacagcgcgtcgtga |
| | | ttagcgccgtggcctgattcaattccccag |
| | | cgaccagtagatcacactcgggtgattacg |
| | | attgcgctgcaccagtcgcgttacggttcg |
| | | ctcttcgccggtagccagcgcggatcacgg |
| | | tcagacgattcgttggcacgatccgtgggt |
| | | ttcaatactggcttcaaaccaccactaaca |
| | | ggccgtagcggtcgcacagcgtgtaccaca |
| | | gcggttggttcggataatcgaacagcgcac |
| | | ggcgttaaagttgttctgcttcaacagcag |
| | | gatattctgcaccttcgtctgctcttccta |
| | | acctgaccaagcagaggatctgctcgtgac |
| | | ggttaatcctcgaatcagcaacggcttgcc |
| | | gttcagcagcagcagaccaagttcaatccg |
| | | cacctcgcggaaaccgacaacgcaggcttc |
| | | tgcttcaatcagcgtgccgtcggcggtgtg |
| | | cagttcaaccaccgcacgatagagattcgg |
| | | gatttcggcgctccacagtttcgggttttc |
| | | gacgttcagacgtagtgtgacgcgatctgc |
| | | aaaccaccacgctcaacgataatttcaccg |
| | | ccgaaaggcgcggtgccgctggcgacctgc |
| | | gtttcaccctgccagaaagaaactgttacc |
| | | cgtaggtagtcacgcaactcgccgcacact |
| | | gaacttcagcctccagtacagcgcggctga |
| | | aatcgtcttaaagcgagtggcaactggaaa |
| | | tcgctgatttgtgtagtcggtttagcagca |
| | | acgagacttcacggaaaatccgctaatccg |
| | | ccacagatcctgatcttccagataactgcc |
| | | gtcactccaacgcagcaccttcaccgcgag |
| | | gcggttttctccggcgcgtaaaaatcgctc |
| | | aggtcaaattcagacggcaaacgactgtcc |
| | | tggccgtaaccgacccagcgcccgttgcac |
| | | cacagattgaaacgccgagtttacgcctca |
| | | aaaataattcgcgtctggccttcctgtagc |
| | | cagctttcacaactataatagtgagcgagt |
| | | aacaacccgtcggattctccgtgggaacaa |
| | | acggcggattgaccgtatagggataggtta |
| | | cgttggtgtagtagggcgctccgtaaccgt |
| | | gctactgccagtttgaggggacgacgacag |
| | | tatcggcctcaggaagatcgcactccagcc |
| | | agctttccggcaccgcttctggtactggaa |
| | | accaggcaaagcgcctatcgcctatcaggc |
| | | tgcacaactgaagggggtagtgctgcaagg |
| | | cgattaagttgggtaacgccagggttttcc |
| | | cagtcacgacgttgtaaaacgacgggatct |
| | | atcagcgctaCATGTTCTTTCCTGCGTTAT |
| | | CCCCTGATTCTGTGGATAACCGTATTACCG |
| | | CCTTTGAGTGAGCTGATACCGCTCAAGGCT |
| | | GACTGCAGGGCGAGAAGATTGCGAGCTGTG |
| | | CGGCTGAGTTGACGTATCTGTGCTGGATGA |
| | | TTACTCATAACGGCACCGCTATCAAACGTG |
| | | CCACGTTCATGTCCTACAGCGCGCTCGCTC |
| | | GCTCACTGAGGCCGCCCGGGCAAAGCCCGG |
| | | GCGTCGGGCGACCTTTGGTCGCCCGGCCTC |
| | | AGTGAGCGAGCGAGCGCGCAGAGAGGGAGT |
| | | GGCCAACTCCATCACTAGGGGTTCCTTGTA |
| | | GTTAATGATTAACCTCTGctagcAGCTGAA |
| | | TGGGGTCCGCCTCTTTTCCCTGCCTAAACA |
| | | GACAGGAACTCCTGCCAATTGAGGGCGTCA |
| | | CCGCTAAGGCTCCGCCCCAGCCTGGGCTCC |
| | | ACAACCAATGAAGGGTAATCTCGACAAAGA |
| | | GCAAGGGGTGGGGCGCGGGCGCGCAGGTGC |
| | | AGCAGCACACAGGCTGGTCGGGAGGGCGGG |
| | | GCGCGACGTCTGCCGTGCGGGGTCCCGGCA |
| | | TCGGTTGCGCGCACCGGTgcgctccctcct |
| | | ctcggagagagggctgtggtaaaacccgtc |
| | | cggaaaTtggccgccgctgccgccaccgcc |
| | | gccgccgccgccgcgccgagcggaggagga |
| | | ggaggaggcgaggaggagagactgtgagtg |
| | | ggaccgccaaggccgcgggcggggaccctt |
| | | gctggggggcgggtaggggctggcgtccct |
| | | cctctctaccctccccctccctctgccgcc |
| | | ggtggtggctttctccactcgtctcccgca |
| | | atcgcgagcgacggttctcagcgcgatctc |
| | | cctggagccaccttcgaTtgacgccctccc |
| | | gctgcccgccccatctgtgcgcatcctagg |
| | | ccccagctgtgcaagcgcccttgtcgtctg |
| | | ggcttcgccatccctcgcgcgcccccactc |
| | | ccgttacttgctcccccctcacacacacag |
| | | actggcgcgcgtgcgcagtccatctcccgt |
| | | tgggagagtgcgccacaagggctcctgagc |
| | | tcttacccccatctctgggttttgctccct |
| | | cctcctcctctcccattccgtgactttttg |
| | | cccccactgcaagcgagtcggtccatcagc |
| | | tccattccccacttggcaggaacaagttga |
| | | gggttattgtccacccacaaaaaggactag |
| | | acattTtgttcctaggtcccacaactcatc |
| | | ataaagagTtggttgtagttctcatcagga |
| | | accgtgggcaagggactgtgcgttcctcag |
| | | cactcgaagctcttccgtgagaccTtgccc |
| | | gcagggtgctctggttctttcccctccctt |
| | | cgccagcgttctgtctacaagaaagaatag |
| | | gcaggtgtccttggatatCgtagttgctaa |
| | | tCgcctatacactgttctattacacctttc |
| | | cagtttggtttagttttggttttggcattt |
| | | agggttttttgggggggagtaatatcttgt |
| | | ggtaaagacccatCtgacccaagatacctt |
| | | ttttctcatactggaaccctaggcagcagt |
| | | tgctatttccctgagttagcaatagtttta |
| | | cagtattttgaggccttttgtccataattc |
| | | tcacggaatCcctcagggatCagattagct |
| | | gctgttgggatCaggaaattgggttacacc |
| | | gctgaaatctgctcacaagttaacagtgcc |
| | | agctgctcttccagaagccctggattcagt |
| | | cccaccaatccatCgcgggtcacaaccatc |
| | | tgtaacttcagtcccaaggggtccgaAgcc |
| | | ctcttctggctttgccctattattttattt |
| | | atcttatCtgtttttgtcttgtcatCtggc |
| | | aagcccagggggccattgggtgcaacttat |
| | | aaactgacttctgtatCttaagaagccaac |
| | | catacagtgcttacattccagaaaaaaaat |
| | | Ctgccactttaacagcactagaactagggt |
| | | ttagagaagtatCataaaggtcaaatatCt |
| | | ttgaccaatatcaccagcaacctaaagctg |
| | | ttaagaaatctttgggccccagcttgaccc |
| | | aaggatacagtatcctagggaagttaccaa |
| | | aatcagagatagtatgcagcagccaggggt |
| | | ctcatgtgtggcactcaagctcacctatac |
| | | tcactactgtgcagacagctgtgttctctg |
| | | taatacttacatatttgtttaatacttcag |
| | | ggaggaaaagtcagaagaccaggatctcca |
| | | gggcctcaACCGGTGGCCCaggCGGCCACC |
| | | ATGTGGACCCTGGTGAGCTGGGTGGCCTTA |
| | | ACAGCAGGGCTGGTGGCTGGAACGCGGTGC |
| | | CCAGATGGTCAGTTCTGCCCTGTGGCCTGC |
| | | TGCCTGGACCCCGGAGGAGCCAGCTACAGC |
| | | TGCTGCCGTCCCCTTCTGGACAAATGGCCC |
| | | ACAACACTGAGCAGGCATCTGGGTGGCCCC |
| | | TGCCAGGTTGATGCCCACTGCTCTGCCGGC |
| | | CACTCCTGCATCTTTACCGTCTCAGGGACT |
| | | TCCAGTTGCTGCCCCTTCCCAGAGGCCGTG |
| | | GCATGCGGGGATGGCCATCACTGCTGCCCA |
| | | CGGGGCTTCCACTGCAGTGCAGACGGGCGA |
| | | TCCTGCTTCCAAAGATCAGGTAACAACTCC |
| | | GTGGGTGCCATCCAGTGCCCTGATAGTCAG |
| | | TTCGAATGCCCGGACTTCTCCACGTGCTGT |
| | | GTTATGGTCGATGGCTCCTGGGGGTGCTGC |
| | | CCCATGCCCCAGGCTTCCTGCTGTGAAGAC |
| | | AGGGTGCACTGCTGTCCGCACGGTGCCTTC |
| | | TGCGACCTGGTTCACACCCGCTGCATCACA |
| | | CCCACGGGCACCCACCCCCTGGCAAAGAAG |
| | | CTCCCTGCCCAGAGGACTAACAGGGCAGTG |
| | | GCCTTGTCCAGCTCGGTCATGTGTCCGGAC |
| | | GCACGGTCCCGGTGCCCTGATGGTTCTACC |
| | | TGCTGTGAGCTGCCCAGTGGGAAGTATGGC |
| | | TGCTGCCCAATGCCCAACGCCACCTGCTGC |
| | | TCCGATCACCTGCACTGCTGCCCCCAAGAC |
| | | ACTGTGTGTGACCTGATCCAGAGTAAGTGC |
| | | CTCTCCAAGGAGAACGCTACCACGGACCTC |
| | | CTCACTAAGCTGCCTGCGCACACAGTGGGG |
| | | GATGTGAAATGTGACATGGAGGTGAGCTGC |
| | | CCAGATGGCTATACCTGCTGCCGTCTACAG |
| | | TCGGGGGCCTGGGGCTGCTGCCCTTTTACC |
| | | CAGGCTGTGTGCTGTGAGGACCACATACAC |
| | | TGCTGTCCCGCGGGGTTTACGTGTGACACG |
| | | CAGAAGGGTACCTGTGAACAGGGGCCCCAC |
| | | CAGGTGCCCTGGATGGAGAAGGCCCCAGCT |
| | | CACCTCAGCCTGCCAGACCCACAAGCCTTG |
| | | AAGAGAGATGTCCCCTGTGATAATGTCAGC |
| | | AGCTGTCCCTCCTCCGATACCTGCTGCCAA |
| | | CTCACGTCTGGGGAGTGGGGCTGCTGTCCA |
| | | ATCCCAGAGGCTGTCTGCTGCTCGGACCAC |
| | | CAGCACTGCTGCCCCCAGGGCTACACGTGT |
| | | GTAGCTGAGGGGCAGTGTCAGCGAGGAAGC |
| | | GAGATCGTGGCTGGACTGGAGAAGATGCCT |
| | | GCCCGCCGGGCTTCCTTATCCCACCCCAGA |
| | | GACATCGGCTGTGACCAGCACACCAGCTGC |
| | | CCGGTGGGGCAGACCTGCTGCCCGAGCCTG |
| | | GGTGGGAGCTGGGCCTGCTGCCAGTTGCCC |
| | | CATGCTGTGTGCTGCGAGGATCGCCAGCAC |
| | | TGCTGCCCGGCTGGCTACACCTGCAACGTG |
| | | AAGGCTCGATCCTGCGAGAAGGAAGTGGTC |
| | | TCTGCCCAGCCTGCCACCTTCCTGGCCCGT |
| | | AGCCCTCACGTGGGTGTGAAGGACGTGGAG |
| | | TGTGGGGAAGGACACTTCTGCCATGATAAC |
| | | CAGACCTGCTGCCGAGACAACCGACAGGGC |
| | | TGGGCCTGCTGTCCCTACCGCCAGGGCGTC |
| | | TGTTGTGCTGATCGGCGCCACTGCTGTCCT |
| | | GCTGGCTTCCGCTGCGCAGCCAGGGGTACC |
| | | AAGTGTTTGCGCAGGGAGGCCCCGCGCTGG |
| | | GACGCCCCTTTGAGGGACCCAGCCTTGAGA |
| | | CAGCTGCTGTGAGGCCAggcCGGCCGaatt |
| | | cGATCCAGACATGATAAGATACATTGATGA |
| | | GTTTGGACAAACCACAACTAGAATGCAGTG |
| | | AAAAAAATGCTTTATTTGTGAAATTTGTGA |
| | | TGCTATTGCTTTATTTGTAACCATTATAAG |
| | | CTGCAATAAACAAGITAACAACAACAATTG |
| | | CATTCATTTTATGTTTCAGGTTCAGGGGGA |
| | | GGTGTGGGAGGTTTTTTAGGGATCCTCAGg |
| | | ttaatcattaactacaaggaacccctagtg |
| | | atggagttggccactccctctctgcgcgct |
| | | cgctcgctcactgaggccgggcgaccaaag |
| | | gtcgcccgacgcccgggctttgcccgggcg |
| | | gcctcagtgagcgagcgagcgcgaACTGTG |
| | | ATTAGCAACTCCTTGTCCTTCGATCTCGTC |
| | | AACAACAGCTTGCAGTTCAAATACAAGACC |
| | | CAGAAGGCGACTATTCTGGAAGCGAGCTTG |
| | | AAGAGTTAACCTGCAGAGAGCCCCCGCAGT |
| | | Gtcgactgttaaccttaattaaccatttaa |
| | | atcgtagtgcaaccgaacgcgaccgttggt |
| | | cagaagccgggcaaatcagcgcctggcagc |
| | | agtggcgtctggCggaaaacctcagtgtga |
| | | cgctccccgccgcgtcccacgcttgttccc |
| | | ggatctgaccaccagcgaaatccgattttt |
| | | gcaccgagctgggtaataagcgttggcaat |
| | | ttaaccgccagtcaggctttctttcacagt |
| | | gtggattggcgataaaaaacaactgctgac |
| | | gccgctgcgcgatcagttcacccgttcacc |
| | | gctggataacgacttggcgtaagtgaagcg |
| | | acccgtaagaccctaacgcctgggtcgaac |
| | | gctggaaggcgggggccaaaccaggccgaa |
| | | gcagcgttgttgcagttcacggcagataca |
| | | cttgctgttgcggtgctgattacgaccgct |
| | | cactcgtggcagcaacaggggaaaacctta |
| | | tttatcagccggaaaacctaccggattgtt |
| | | ggtagtggtcaataggcgattaccgttgtg |
| | | ttgaagtggcgagcgatacaccgcttccgg |
| | | cgcggattggcctgaactgccaactggcgc |
| | | aggtagcagagcgggtaaactggctcggat |
| | | tagggccgcaagaaaactatcccgaccgcc |
| | | ttactgccgcctgttttgaccgctgggatc |
| | | tgccaagtcagacagtatagcccgtacgtc |
| | | ttcccgagcgaaaacggtctgcgctgcggg |
| | | acgcgcgaattgaatttggcccacaccagt |
| | | ggcgcggcgacttccagttcaatatcagcc |
| | | gctacagtgaacagcaactgttggaaacca |
| | | gccttcgccaactgctgcacgcggaagaag |
| | | gcactggctgaatatcgacggtttccagtt |
| | | ggggattggtggcgacgactcctggagccg |
| | | tcagtatcggcggacttccaactgagcgcc |
| | | ggtcgctaccttaccagttggtctggtgtc |
| | | aaaaagcgtccgcttgagtctagcgatcgc |
| | | gcgcagatctgtcatgtgagcaaaaggcca |
| | | gcaaaaggccaggaaccgtaaaaaggccgc |
| | | gttgctggcgtttttccataggctccgccc |
| | | ccctgacgagcatcacaaaaatcgacgctc |
| | | aagtcagaggtggcgaaacccgacaggact |
| | | ataaagataccaggcgtttccccctggaag |
| | | ctccctcgtgcgctctcctgttccgaccct |
| | | gccgcttaccggatacctgtccgcctttct |
| | | cccttcgggaagcgtggcgctttctcatag |
| | | ctcacgctgtaggtatctcagttcggtgta |
| | | ggtcgttcgctccaagctgggctgtgtgca |
| | | cgaaccccccgttcagcccgaccgctgcgc |
| | | cttatccggtaactatcgtcttgagtccaa |
| | | cccggtaagacacgacttatcgccactggc |
| | | agcagccactggtaacaggattagcagagc |
| | | gaggtatgtaggcggtgctacagagttctt |
| | | gaagtggtggcctaactacggctacactag |
| | | aagaacagtatttggtatctgcgctctgct |
| | | gaagccagttaccttcggaaaaagagttgg |
| | | tagctcttgatccggcaaacaaaccaccgc |
| | | tggtagcggtggtttttttgtttgcaagca |
| | | gcagattacgcgcagaaaaaaaggatctca |
| | | agaagatcctttgatcttttctacggggtc |
| | | tgacgctcagtggaacgaaaactcacgtta |
| | | agggattttggtcatgagattatcaaaaag |
| | | gatcttcacctagatccttttcacgtagaa |
| | | agccagtccgcagaaacggtgctgaccccg |
| | | gatgaatgtcagctactgggctatctggac |
| | | aagggaaaacgcaagcgcaaagagaaagca |
| | | ggtagcttgcagtgggcttacatggcgata |
| | | gctagactgggcggttttatggacagcaag |
| | | cgaaccggaattgccagctggggcgccctc |
| | | tggtaaggttgggaagccctgcaaagtaaa |
| | | ctggatggctttcttgccgccaaggatctg |
| | | atggcgcaggggatcaagatctgatcaaga |
| | | gacaggatgaggatcgtttcgcatgattga |
| | | acaagatggattgcacgcaggttctccgcg |
| | | gctgctctgatgccgccgtgttccggctgt |
| | | cagcgcagggcgcccgggttctttttgtca |
| | | agaccgacctgtccggtgccctgaatgaac |
| | | tgcaagacgaggcagcgcggctatcgtggc |
| | | tggccacgacgggcgttccttgcgcagctg |
| | | tgctcgacgttgtcactgaagcgggaaggg |
| | | actggctgctattgggcgaagtgccggggc |
| | | aggatctcctgtcatctcaccttgctcctg |
| | | ccgagaaagtatccatcatggctgatgcaa |
| | | tgcggcggctgcatacgcttgatccggcta |
| | | cctgcccattcgaccaccaagcgaaacatc |
| | | gcatcgagcgagcacgtactcggatggaag |
| | | ccggtcttgtcgatcaggatgatctggacg |
| | | aagagcatcaggggctcgcgccagccgaac |
| | | tgttcgccaggctcaaggcgagcatgcccg |
| | | acggcgaggatctcgtcgtgacccatggcg |
| | | atgcctgcttgccgaatatcatggtggaaa |
| | | atggccgcttttctggattcatcgactgtg |
| | | gccggctgggtgtggcggaccgctatcagg |
| | | acatagcgttggctacccgtgatattgctc |
| | | gccgctcccgattcgcagcgcatcgccttc |
| | | tatcgccttcttgacgagttcttctgaatt |
| | | taaagcccaatacgcaaaccgcctctcccc |
| | | gcgcgttggcc |
| 20 | 5′ ITR | GCGCGCTCGCTCGCTCACTGAGGCCGCCCG |
| | | GGCAAAGCCCGGGCGTCGGGCGACCTTTGG |
| | | TCGCCCGGCCTCAGTGAGCGAGCGAGCGCG |
| | | CAGAGAGGGAGTGGCCAACTCCATCACTAG |
| | | GGGTTCCT |
| 21 | 5′ adjacent | TGTAGTTAATGATTAACC |
| | fragment | |
| 22 | 3′ adjacent | GTTAATCATTAACTACA |
| | fragment | |
| 23 | 3′ ITR | AGGAACCCCTAGTGATGGAGTTGGCCACTC |
| | | CCTCTCTGCGCGCTCGCTCGCTCACTGAGG |
| | | CCGGGCGACCAAAGGTCGCCCGACGCCCGG |
| | | GCTTTGCCCGGGCGGCCTCAGTGAGCGAGC |
| | | GAGCGCGC |
| 24 | Kozak | GCCACC |
| | sequence | |

Further aspects of the present invention:
1. A nucleic acid construct comprising a methyl CpG binding protein 2 (MeCP2) promoter operably linked to a nucleotide sequence encoding a progranulin (PGRN) protein.
2. The nucleic acid construct of paragraph 1, wherein the MeCP2 promoter is an engineered MeCP2 promoter comprising a minimal promoter sequence and at least one intron.
3. A nucleic acid construct comprising an engineered methyl CpG binding protein 2 (MeCP2) promoter operably linked to a nucleotide sequence encoding a protein of interest (POI), wherein the engineered MeCP2 promoter comprises a minimal promoter sequence and at least one intron.
4. The nucleic acid construct of paragraph 3, wherein the POI is a progranulin (PGRN) protein.
5. The nucleic acid construct of any one of paragraphs 2-4, wherein: (a) the at least one intron is 3′ to the minimal promoter sequence; or (b) the at least one intron is 5′ to minimal promoter sequence.
6. The nucleic acid construct of any one of paragraphs 2-5, wherein the at least one intron is synthetic.
7. The nucleic acid construct of paragraph 6, wherein the at least one synthetic intron comprises one or more nucleotide sequences of an MECP2 gene, optionally wherein the at least one synthetic intron comprises one or more intronic sequences of an MECP2 gene and/or one or more non-expressing exonic sequences of an MECP2 gene, preferably wherein the MECP2 gene is a human MECP2 gene.
8. The nucleic acid construct of paragraph 6 or 7, wherein the at least one synthetic intron comprises two intronic sequences of a human MECP2 gene and two non-expressing exonic sequences of a human MECP2 gene.
9. The nucleic acid construct of any one of paragraphs 6-8, wherein the at least one synthetic intron comprises:
   (a) a non-expressing exonic sequence comprising the nucleotide sequence of SEQ ID NO: 4 or a nucleotide sequence having at least 90% identity to SEQ ID NO: 4;
   (b) an intronic sequence comprising the nucleotide sequence of SEQ ID NO: 5 or a nucleotide sequence having at least 90% identity to the nucleotide sequence of SEQ ID NO: 5;
   (c) an intronic sequence comprising the nucleotide sequence of SEQ ID NO: 6 or a nucleotide sequence having at least 90% identity to the nucleotide sequence of SEQ ID NO: 6; and/or
   (d) a non-expressing exonic sequence comprising the nucleotide sequence of SEQ ID NO: 7 or a nucleotide sequence having at least 90% identity to the nucleotide sequence of SEQ ID NO: 7.
10. The nucleic acid construct of any one of paragraphs 6-9, wherein, in the 5′ to 3′ direction, the at least one synthetic intron comprises:
   (a) a non-expressing exonic sequence comprising the nucleotide sequence of SEQ ID NO: 4 or a nucleotide sequence having at least 90% identity to the nucleotide sequence of SEQ ID NO: 4;
   (b) an intronic sequence comprising the nucleotide sequence of SEQ ID NO: 5 or a nucleotide sequence having at least 90% identity to the nucleotide sequence of SEQ ID NO: 5;
   (c) an intronic sequence comprising the nucleotide sequence of SEQ ID NO: 6 or a nucleotide sequence having at least 90% identity to the nucleotide sequence of SEQ ID NO: 6; and
   (d) a non-expressing exonic sequence comprising the nucleotide sequence of SEQ ID NO: 7 or a nucleotide sequence having at least 90% identity to the nucleotide sequence of SEQ ID NO: 7.
11. The nucleic acid construct of any one of paragraphs 6-10, wherein the at least one synthetic intron comprises the nucleotide sequence of SEQ ID NO: 2 or a nucleotide sequence having at least 90% identity to the nucleotide sequence of the nucleotide sequence of SEQ ID NO: 2.
12. The nucleic acid construct of any one of paragraphs 2-5, wherein the at least one intron is a natural intron.
13. The nucleic acid construct of paragraph 12, wherein the at least one natural intron comprises a nucleotide sequence of an MeCP2 gene, preferably a human MeCP2 gene.
14. The nucleic acid construct of paragraph 13, wherein the at least one natural intron comprises the nucleotide sequence of SEQ ID NO: 9 or a nucleotide sequence having at least 90% identity to the nucleotide sequence of SEQ ID NO: 9.
15. The nucleic acid construct of any one of paragraphs 2-14, wherein the minimal promoter sequence comprises the nucleotide sequence of SEQ ID NO: 1, or a functional variant or fragment thereof having at least 90% identity to the nucleotide sequence of SEQ ID NO: 1.
16. The nucleic acid construct of any one of paragraphs 1-11, wherein the engineered MeCP2 promoter comprises the nucleotide sequence of SEQ ID NO: 3 or a functional variant or fragment thereof having at least 90% identity to the nucleotide sequence of SEQ ID NO: 3.
17. The nucleic acid construct of any one of paragraphs 1-5 or 12-14, wherein the engineered MeCP2 promoter comprises the nucleotide sequence of SEQ ID NO: 8 or a functional variant or fragment thereof having at least 90% identity to the nucleotide sequence of SEQ ID NO: 8.
18. The nucleic acid sequence of any one of paragraphs 1-17, wherein the MeCP2 promoter is at least about 1000 bp, 1500 bp, 2000 bp, 2100 bp, 2150 bp, 2175 bp, 2200 bp, 2210 bp, 2220 bp, 2230 bp, 2240 bp, 2250 bp, 2260 bp, 2280 bp, 2290 bp, 2300 bp, 2310 bp, 2320, 2330 bp in length, preferably wherein the MeCP2 promoter is about 2200-2350 bp in length.
19. The nucleic acid construct of any one of paragraphs 1, 2 or 4-18, wherein:
   (a) the PGRN protein is a human PGRN protein;
   (b) the PGRN protein is a wild type protein;
   (c) the nucleotide sequence encoding the PGRN protein is a human nucleotide sequence;
   (d) the nucleotide sequence encoding the PGRN proteins is a wild type nucleotide sequence;
   (e) the nucleotide sequence encoding the PGN protein is not codon optimised; and/or
   (f) the nucleotide sequence encoding the PGRN protein is at least about 1600 bp, 1700 bp, 1750 bp, 1760 bp, 1770 bp, or 1780 bp, preferably wherein the nucleotide sequence encoding the PGRN protein is about 1780 bp in length.
20. The nucleic acid construct of any one of paragraphs 1, 2 or 4-19, wherein:
   the nucleotide sequence encoding the PGRN protein comprises the nucleotide sequence of SEQ ID NO: 12 or a functional variant or fragment thereof having at least 70% identity to the nucleotide sequence of SEQ ID NO: 12; and/or
   the PGRN protein comprises the amino acid sequence of SEQ ID NO: 13 or a functional variant or fragment thereof having at least 70% identity to the amino acid sequence of SEQ ID NO: 13.
21. The nucleic acid construct of any one of paragraphs 1-20, which further comprises:
   (a) a woodchuck hepatitis virus (WHP) posttranscriptional regulatory element (WPRE) sequence, optionally wherein the WPRE is 3′ to the nucleotide sequence encoding the POI or the PGRN protein and/or the WPRE comprises the nucleotide sequence of SEQ ID NO: 15 or a functional variant or fragment thereof having at least 90% identity to the nucleotide sequence of SEQ ID NO: 15;
   (b) a polyadenylation signal sequence, optionally wherein the polyadenylation signal sequence is 3′ to the nucleotide sequence encoding the POI or the PGRN protein and/or the polyadenylation signal sequence comprises the nucleotide sequence SEQ ID NO: 16 or a functional variant or fragment thereof having at least 90% identity to the nucleotide sequence of SEQ ID NO: 16; or
   (c) (a) and (b) above, optionally wherein, in the 5′ to 3′ direction, the nucleic acid construct comprises the MeCP2 promoter, the nucleotide sequence encoding the POI or the PGRN protein, the WPRE, and the polyadenylation signal sequence.
22. The nucleic acid construct of any one of paragraphs 1-21, which is 3700 to 4700 bp, 3800 to 4800 bp, 3900 to 4700 bp, 4000 to 4600 bp, 4000 to 4500 bp, 4000 to 4400 bp, 4000 to 4300 bp, or 4000 to 4200 bp in length.
23. A vector comprising a nucleic acid construct as defined in any one of paragraphs 1-22.
24. The vector of paragraph 23, which is a plasmid or a viral vector.
25. The vector of paragraph 23 or 24, which is a viral vector comprising the nucleotide sequence of:
   (a) SEQ ID NO: 11 or a functional variant or fragment thereof having at least 70% identity to the nucleotide sequence of SEQ ID NO: 11; or
   (b) SEQ ID NO: 10 or a functional variant or fragment thereof having at least 70% identity to the nucleotide sequence of SEQ ID NO: 10.
26. The vector of paragraph any one of paragraphs 23-25, which is a viral vector selected from: (a) an adeno-associated virus (AAV) vector or which comprises an AAV genome or a derivative thereof, optionally wherein said derivative is a chimeric, shuffled or capsid modified derivative; or (b) a lentiviral vector or which comprises a lentivirus genome or a derivative thereof.
27. The viral vector of paragraph 26, which is an AAV vector comprising a genome derived from AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), or AAV serotype rhl0 (AAVrhl0), preferably wherein the AAV comprises a genome derived from AAV2, AAV9 or AAVrH10.
28. The AAV vector of paragraph 27, wherein the AAV vector comprises a genome derived from AAV2, preferably wherein the AAV is AAV-TT.
29. A host cell which comprises a nucleic acid construct according to any one of paragraphs 1-22 and/or a vector according to any one of paragraphs 23-28, and/or which produces a viral vector according to any one of paragraphs 25-28, optionally wherein the host cell is a HEK293 cell or a HEK293T cell.
30. A pharmaceutical composition comprising a nucleic acid construct according to any one of paragraphs 1-22, a vector according to paragraph 23 or 24, and/or a viral vector according to any one of paragraphs 25-28 together with a pharmaceutically acceptable carrier, excipient or diluent.
31. A nucleic acid construct as defined in any one of paragraphs 1-22, a vector as defined in paragraph 23 or 24, a viral vector as defined in any one of paragraphs 25-28, and/or a pharmaceutical composition as defined in paragraph 30 for use in a method of treating or preventing a disease characterized by progranulin (PGRN) deficiency in a patient in need thereof.
32. A method of treating or preventing a disease characterized by progranulin (PGRN) deficiency in a patient in need thereof, said method comprising administering to the patient a therapeutically effective amount of a nucleic acid construct as defined in any one of paragraphs 1-22, a vector as defined in paragraph 23 or 24, a viral vector as defined in any one of paragraphs 25-28, and/or a pharmaceutical composition as defined in paragraph 30.
33. Use of a nucleic acid construct as defined in any one of paragraphs 1-22, a vector as defined in paragraph 23 or 24, a viral vector as defined in any one of paragraphs 25-28, and/or a pharmaceutical composition as defined in paragraph 30 for the manufacture of a medicament for the treatment or prevention of a disease characterized by progranulin (PGRN) deficiency in a patient in need thereof.
34. The nucleic acid construct, vector, viral vector, or pharmaceutical composition for use according to paragraph 31, the method of paragraph 32, or the use of paragraph 33, wherein:
   the disease characterized by PGRN deficiency is a disease of the central nervous system;
   the disease characterized by PGRN deficiency is characterized by a deficiency of PGRN in the neurons and/or the astrocytes of the patient;
   the patient has a loss of function mutation in at least one allele of their GRN gene; and/or
   the patient has a loss of function mutation in both alleles of their GRN gene.
35. The nucleic acid construct, vector, viral vector, or pharmaceutical composition for use according to paragraph 31 or 34, the method of paragraph 32 or 34, or the use of paragraph 33 or 34, wherein the disease characterized by PGRN deficiency is frontotemporal dementia (FTD) or neuronal ceroid lipofuscinosis type 11 (NCL11).
36. The nucleic acid construct, vector, viral vector, or pharmaceutical composition for use according to paragraph 31, 34 or 35, the method of paragraph 32, 34 or 35, or the use of paragraph any one of paragraphs 33-35, wherein said nucleic acid construct, vector, viral vector, or pharmaceutical composition are administered to the patient by delivery to the brain and/or the cerebrospinal fluid (CSF) of the patient, optionally wherein the delivery is by injection to:
   (i) the brain of the patient, preferably wherein the injection to the brain is selected from intracerebral injection, intraparenchymal injection, intraputaminal injection, and combinations thereof; and/or
   (ii) the CSF of the patient, preferably wherein the injection to the CSF is selected from intra-cisterna magna injection, intrathecal injection, intracerebroventricular (ICV) injection, and combinations thereof.

1-41. (canceled)

42. A nucleic acid construct comprising a methyl CpG binding protein 2 (MeCP2) promoter operably linked to a nucleotide sequence encoding a progranulin (PGRN) protein.

43. The nucleic acid construct of claim 42, wherein the MeCP2 promoter is an engineered MeCP2 promoter comprising a minimal promoter sequence and at least one intron.

44. The nucleic acid construct of claim 43, wherein: (a) the at least one intron is 3′ to the minimal promoter sequence; or (b) the at least one intron is 5′ to minimal promoter sequence.

45. The nucleic acid construct of claim 43, wherein the at least one intron is synthetic.

46. The nucleic acid construct of claim 45, wherein the at least one synthetic intron comprises one or more nucleotide sequences of an MECP2 gene, optionally wherein the at least one synthetic intron comprises one or more intronic sequences of an MECP2 gene and/or one or more non-expressing exonic sequences of an MECP2 gene.

47. The nucleic acid construct of claim 45, wherein the at least one synthetic intron comprises two intronic sequences of a murine MECP2 gene and two non-expressing exonic sequences of a murine MECP2 gene.

48. The nucleic acid construct of claim 45, wherein the at least one synthetic intron comprises:
(a) a non-expressing exonic sequence comprising the nucleotide sequence of SEQ ID NO: 4 or a nucleotide sequence having at least 90% identity to SEQ ID NO: 4;
(b) an intronic sequence comprising the nucleotide sequence of SEQ ID NO: 5 or a nucleotide sequence having at least 90% identity to the nucleotide sequence of SEQ ID NO: 5;
(c) an intronic sequence comprising the nucleotide sequence of SEQ ID NO: 6 or a nucleotide sequence having at least 90% identity to the nucleotide sequence of SEQ ID NO: 6; and/or
(d) a non-expressing exonic sequence comprising the nucleotide sequence of SEQ ID NO: 7 or a nucleotide sequence having at least 90% identity to the nucleotide sequence of SEQ ID NO: 7.

49. The nucleic acid construct of claim 45, wherein, in the 5′ to 3′ direction, the at least one synthetic intron comprises:
(a) a non-expressing exonic sequence comprising the nucleotide sequence of SEQ ID NO: 4 or a nucleotide sequence having at least 90% identity to the nucleotide sequence of SEQ ID NO: 4;
(b) an intronic sequence comprising the nucleotide sequence of SEQ ID NO: 5 or a nucleotide sequence having at least 90% identity to the nucleotide sequence of SEQ ID NO: 5;
(c) an intronic sequence comprising the nucleotide sequence of SEQ ID NO: 6 or a nucleotide sequence having at least 90% identity to the nucleotide sequence of SEQ ID NO: 6; and
(d) a non-expressing exonic sequence comprising the nucleotide sequence of SEQ ID NO: 7 or a nucleotide sequence having at least 90% identity to the nucleotide sequence of SEQ ID NO: 7.

50. The nucleic acid construct of claim 48, wherein the at least one synthetic intron comprises the nucleotide sequence of SEQ ID NO: 2 or a nucleotide sequence having at least 90% identity to the nucleotide sequence of the nucleotide sequence of SEQ ID NO: 2.

51. The nucleic acid construct of claim 49, wherein the at least one synthetic intron comprises the nucleotide sequence of SEQ ID NO: 2 or a nucleotide sequence having at least 90% identity to the nucleotide sequence of the nucleotide sequence of SEQ ID NO: 2.

52. The nucleic acid construct of claim 42, wherein the engineered MeCP2 promoter comprises the nucleotide sequence of SEQ ID NO: 3 or a functional variant or fragment thereof having at least 90% identity to the nucleotide sequence of SEQ ID NO: 3.

53. The nucleic acid construct of claim 42, which further comprises:
(a) a woodchuck hepatitis virus (WHP) posttranscriptional regulatory element (WPRE) sequence, optionally wherein the WPRE is 3′ to the nucleotide sequence encoding the POI or the PGRN protein and/or the WPRE comprises the nucleotide sequence of SEQ ID NO: 15 or a functional variant or fragment thereof having at least 90% identity to the nucleotide sequence of SEQ ID NO: 15;
(b) a polyadenylation signal sequence, optionally wherein the polyadenylation signal sequence is 3′ to the nucleotide sequence encoding the POI or the PGRN protein and/or the polyadenylation signal sequence comprises the nucleotide sequence SEQ ID NO: 16 or a functional variant or fragment thereof having at least 90% identity to the nucleotide sequence of SEQ ID NO: 16; or
(c) (a) and (b) above, optionally wherein, in the 5′ to 3′ direction, the nucleic acid construct comprises the MeCP2 promoter, the nucleotide sequence encoding the POI or the PGRN protein, the WPRE, and the polyadenylation signal sequence.

54. The nucleic acid construct of claim 42, which is 3700 to 4700 bp in length.

55. A nucleic acid construct comprising an engineered methyl CpG binding protein 2 (MeCP2) promoter operably linked to a nucleotide sequence encoding a protein of interest (POI), wherein the engineered MeCP2 promoter comprises a minimal promoter sequence and at least one intron.

56. The nucleic acid construct of claim 55, wherein the POI is a progranulin (PGRN) protein.

57. A vector comprising a nucleic acid construct according to claim 42.

58. The vector of claim 57, which is a viral vector selected from: (a) an adeno-associated virus (AAV) vector or which comprises an AAV genome or a derivative thereof, optionally wherein said derivative is a chimeric, shuffled or capsid modified derivative; or (b) a lentiviral vector or which comprises a lentivirus genome or a derivative thereof.

59. The AAV vector of claim 58, wherein the AAV vector comprises a nucleotide sequence which, in the 5′ to 3′ direction, comprises one or more of:
(a) a 5′ ITR;
(b) a 5′ adjacent fragment;
(c) a minimal MeCP2 promoter sequence;
(d) at least one synthetic intron;
(e) a Kozak sequence;
(f) a polynucleotide sequence encoding a PGRN protein;
(g) an SV40 poly(A) sequence;
(h) a 3′ adjacent fragment; and
(i) a 3′ ITR.

60. The AAV vector of claim 59, wherein:
(a) the 5′ ITR comprises or consists of the nucleotide sequence of SEQ ID NO: 20 or a functional variant or fragment thereof having at least 70% identity to SEQ ID NO: 20;
(b) the 5′ adjacent fragment comprises or consists of the nucleotide sequence of SEQ ID NO: 21 or a functional variant or fragment thereof having at least 70% identity to SEQ ID NO: 21;
(c) the minimal MeCP2 promoter sequence comprises or consists of the nucleotide sequence of SEQ ID NO: 1 or a functional variant or fragment thereof having at least 70% identity to SEQ ID NO: 1;
(d) the at least one synthetic intron comprises or consists of the nucleotide sequence of SEQ ID NO: 2 or a functional variant or fragment thereof having at least 70% identity to SEQ ID NO: 2;
(e) the Kozak sequence comprises or consist of the nucleotide sequence of SEQ ID NO: 24;
(f) the polynucleotide sequence encoding a PGRN protein comprises or consists of the nucleotide sequence of SEQ ID NO: 12 or a functional variant or fragment thereof having at least 70% identity to SEQ ID NO: 12;
(g) the SV40 poly(A) sequence comprises or consists of the nucleotide sequence of SEQ ID NO: 16 or a functional variant or fragment thereof having at least 70% identity to SEQ ID NO: 16;
(h) the 3′ adjacent fragment comprises or consists of the nucleotide sequence of SEQ ID NO: 22 or a functional variant or fragment thereof having at least 70% identity to SEQ ID NO: 22; and/or
(i) the 3′ ITR comprises or consists of the nucleotide sequence of SEQ ID NO: 23 or a functional variant or fragment thereof having at least 70% identity to SEQ ID NO: 23.

61. A host cell which comprises a nucleic acid construct according to claim 42.

62. A pharmaceutical composition comprising a nucleic acid construct according to claim 42 together with a pharmaceutically acceptable carrier, excipient or diluent.

63. A method of treating or preventing a disease characterized by progranulin (PGRN) deficiency in a patient in need thereof, said method comprising administering to the patient a therapeutically effective amount of a nucleic acid construct as defined in claim 42.

64. The method of claim 63, wherein:
the disease characterized by PGRN deficiency is a disease of the central nervous system;
the disease characterized by PGRN deficiency is characterized by a deficiency of PGRN in the neurons and/or the astrocytes of the patient;
the patient has a loss of function mutation in at least one allele of their GRN gene; and/or
the patient has a loss of function mutation in both alleles of their GRN gene.

65. The method of claim 63, wherein the disease characterized by PGRN deficiency is frontotemporal dementia (FTD) or neuronal ceroid lipofuscinosis type 11 (NCL11).

66. The method of claim 63, wherein the nucleic acid construct is administered to the patient by delivery to the brain and/or the cerebrospinal fluid (CSF) of the patient.


SEQUENCE LISTING (24 sequences)

SEQ ID NO: 1 - DNA, 229 bp, Artificial Sequence
  MeCP2 minimal promoter
agctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgc

SEQ ID NO: 2 - DNA, 2000 bp, Artificial Sequence
  MeCP2_2 intron
gcgctccctcctctcggagagagggctgtggtaaaacccgtccggaaattggccgccgctgccgccaccgccgccgccgccgccgcgccgagcggaggaggaggaggaggcgaggaggagagactgtgagtgggaccgccaaggccgcgggcggggacccttgctggggggcgggtaggggcgggacgtggcgcgggaggggcccgcggggtcgggcgacacggctggcggttggcgtccctcctctctaccctccccctccctctgccgccggtggtggctttctccactcgtctcccgcaatcgcgagcgacggttctcagcgcgatctccctggagccaccttcgattgacgccctcccgctgcccgccccatctgtgcgcatcctaggccccagctgtgcaagcgcccttgtcgtctgggcttcgccagttggggctgcgcgcgctcctgcccttcttggggctttgggcctcggcactgtcgcgcgcccgcggtcccggcctctccctggatcgcgctgtccccttctccctcgcgcgcccccactcccgttacttgctcccccctcacacacacagactggcgcgcgtgcgcagtccatctcccgttgggagagtgcgccacaagggctcctgagctcttacccccatctctgggttttgctccctcctcctcctctcccattccgtgactttttgcccccactgcaagcgagtcggtccatcagctccattccccacttggcaggaacaagttgagggttattgtccacccacaaaaaggactagacattttgttcctaggtcccacaactcatcataaagagttggttgtagttctcatcaggaaccgtgggcaagggactgtgcgttcctcagcactcgaagctcttccgtgagaccttgcccgcagggtgctctggttctttggggttgctgtgctgtggcttcggaatttgagcgtcttcccaccctccctcccctcccttcgccagcgttctgtctacaagaaagaataggcaggtgtccttggatatcgtagttgctaatcgcctatacactgttctattacacctttctgctaaggatagggtttttggttttggttttggttttgttccccaccctccagtttggtttagttttggttttggcatttagggttttttgggggggagtaatatcttgtggtaaagacccatctgacccaagataccttttttctcatactggaaccctaggcagcagttgctatttccctgagttagcaatagttttacagtattttgaggccttttgtccataattctcacggaatccctcagggatcagattagctgctgttgggatcaggaaattgggttacaccgctgaaatctcttgctggggcccttgttttgaattggaaagtcaggaggctggaacgaaggctcacaagttaacagtgccagctgctcttccagaagccctggattcagtcccaccaatccatcgcgggtcacaaccatctgtaacttcagtcccaaggggtccgaagccctcttctggctttgccctattattttatttatcttatctgtttttgtcttgtcatctggcaagcccagggggccattgggtgcaacttataaactgacttctgtatcttaagaagccaaccatacagtgcttacattccagaaaaaaaatctgccactttaacagcactagaactagggtttagagaagtatcataaaggtcaaatatctttgaccaatatcaccagcaacctaaagctgttaagaaatctttgggccccagcttgacccaaggatacagtatcctagggaagttaccaaaatcagagatagtatgcagcagccaggggtctcatgtgtggcactcaagctcacctatactcactactgtgcagacagctgtgttctctgtaatacttacatatttgtttaatacttcagggaggaaaagtcagaagaccaggatctccagggcctca

SEQ ID NO: 3 - DNA, 2235 bp, Artificial Sequence
  MeCP2_2 promoter
agctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcaccggtgcgctccctcctctcggagagagggctgtggtaaaacccgtccggaaattggccgccgctgccgccaccgccgccgccgccgccgcgccgagcggaggaggaggaggaggcgaggaggagagactgtgagtgggaccgccaaggccgcgggcggggacccttgctggggggcgggtaggggcgggacgtggcgcgggaggggcccgcggggtcgggcgacacggctggcggttggcgtccctcctctctaccctccccctccctctgccgccggtggtggctttctccactcgtctcccgcaatcgcgagcgacggttctcagcgcgatctccctggagccaccttcgattgacgccctcccgctgcccgccccatctgtgcgcatcctaggccccagctgtgcaagcgcccttgtcgtctgggcttcgccagttggggctgcgcgcgctcctgcccttcttggggctttgggcctcggcactgtcgcgcgcccgcggtcccggcctctccctggatcgcgctgtccccttctccctcgcgcgcccccactcccgttacttgctcccccctcacacacacagactggcgcgcgtgcgcagtccatctcccgttgggagagtgcgccacaagggctcctgagctcttacccccatctctgggttttgctccctcctcctcctctcccattccgtgactttttgcccccactgcaagcgagtcggtccatcagctccattccccacttggcaggaacaagttgagggttattgtccacccacaaaaaggactagacattttgttcctaggtcccacaactcatcataaagagttggttgtagttctcatcaggaaccgtgggcaagggactgtgcgttcctcagcactcgaagctcttccgtgagaccttgcccgcagggtgctctggttctttggggttgctgtgctgtggcttcggaatttgagcgtcttcccaccctccctcccctcccttcgccagcgttctgtctacaagaaagaataggcaggtgtccttggatatcgtagttgctaatcgcctatacactgttctattacacctttctgctaaggatagggtttttggttttggttttggttttgttccccaccctccagtttggtttagttttggttttggcatttagggttttttgggggggagtaatatcttgtggtaaagacccatctgacccaagataccttttttctcatactggaaccctaggcagcagttgctatttccctgagttagcaatagttttacagtattttgaggccttttgtccataattctcacggaatccctcagggatcagattagctgctgttgggatcaggaaattgggttacaccgctgaaatctcttgctggggcccttgttttgaattggaaagtcaggaggctggaacgaaggctcacaagttaacagtgccagctgctcttccagaagccctggattcagtcccaccaatccatcgcgggtcacaaccatctgtaacttcagtcccaaggggtccgaagccctcttctggctttgccctattattttatttatcttatctgtttttgtcttgtcatctggcaagcccagggggccattgggtgcaacttataaactgacttctgtatcttaagaagccaaccatacagtgcttacattccagaaaaaaaatctgccactttaacagcactagaactagggtttagagaagtatcataaaggtcaaatatctttgaccaatatcaccagcaacctaaagctgttaagaaatctttgggccccagcttgacccaaggatacagtatcctagggaagttaccaaaatcagagatagtatgcagcagccaggggtctcatgtgtggcactcaagctcacctatactcactactgtgcagacagctgtgttctctgtaatacttacatatttgtttaatacttcagggaggaaaagtcagaagaccaggatctccagggcctca

SEQ ID NO: 4 - DNA, 125 bp, Artificial Sequence
  MeCP2_2 intron - exon1
gcgctccctcctctcggagagagggctgtggtaaaacccgtccggaaattggccgccgctgccgccaccgccgccgccgccgccgcgccgagcggaggaggaggaggaggcgaggaggagagact

SEQ ID NO: 5 - DNA, 875 bp, Artificial Sequence
  MeCP2_2 intron - 5′ intron
gtgagtgggaccgccaaggccgcgggcggggacccttgctggggggcgggtaggggcgggacgtggcgcgggaggggcccgcggggtcgggcgacacggctggcggttggcgtccctcctctctaccctccccctccctctgccgccggtggtggctttctccactcgtctcccgcaatcgcgagcgacggttctcagcgcgatctccctggagccaccttcgattgacgccctcccgctgcccgccccatctgtgcgcatcctaggccccagctgtgcaagcgcccttgtcgtctgggcttcgccagttggggctgcgcgcgctcctgcccttcttggggctttgggcctcggcactgtcgcgcgcccgcggtcccggcctctccctggatcgcgctgtccccttctccctcgcgcgcccccactcccgttacttgctcccccctcacacacacagactggcgcgcgtgcgcagtccatctcccgttgggagagtgcgccacaagggctcctgagctcttacccccatctctgggttttgctccctcctcctcctctcccattccgtgactttttgcccccactgcaagcgagtcggtccatcagctccattccccacttggcaggaacaagttgagggttattgtccacccacaaaaaggactagacattttgttcctaggtcccacaactcatcataaagagttggttgtagttctcatcaggaaccgtgggcaagggactgtgcgttcctcagcactcgaagctcttccgtgagaccttgcccgcagggtgctctggttctttggggttgctgtgctgtggcttcggaatttgagcgtcttcccaccctccctcccctcccttcgccagcg

SEQ ID NO: 6 - DNA, 962 bp, Artificial Sequence
  MeCP2_2 intron - 3′ intron
ttctgtctacaagaaagaataggcaggtgtccttggatatcgtagttgctaatcgcctatacactgttctattacacctttctgctaaggatagggtttttggttttggttttggttttgttccccaccctccagtttggtttagttttggttttggcatttagggttttttgggggggagtaatatcttgtggtaaagacccatctgacccaagataccttttttctcatactggaaccctaggcagcagttgctatttccctgagttagcaatagttttacagtattttgaggccttttgtccataattctcacggaatccctcagggatcagattagctgctgttgggatcaggaaattgggttacaccgctgaaatctcttgctggggcccttgttttgaattggaaagtcaggaggctggaacgaaggctcacaagttaacagtgccagctgctcttccagaagccctggattcagtcccaccaatccatcgcgggtcacaaccatctgtaacttcagtcccaaggggtccgaagccctcttctggctttgccctattattttatttatcttatctgtttttgtcttgtcatctggcaagcccagggggccattgggtgcaacttataaactgacttctgtatcttaagaagccaaccatacagtgcttacattccagaaaaaaaatctgccactttaacagcactagaactagggtttagagaagtatcataaaggtcaaatatctttgaccaatatcaccagcaacctaaagctgttaagaaatctttgggccccagcttgacccaaggatacagtatcctagggaagttaccaaaatcagagatagtatgcagcagccaggggtctcatgtgtggcactcaagctcacctatactcactactgtgcagacagctgtgttctctgtaatacttacatatttgtttaatacttcag

SEQ ID NO: 7 - DNA, 38 bp, Artificial Sequence
  MeCP2_2 intron - exon2
ggaggaaaagtcagaagaccaggatctccagggcctca

SEQ ID NO: 8 - DNA, 2337 bp, Artificial Sequence
  MeCP2_1 promoter
ctctaccattacgttttatcctcagactctatctccccattttaaaggaatattatttttaaatgctacactctcattttttaaatggctccttttaattctactgctaaaatacttttggtacaatatgcctttttttctattttttttttttagtgcaagtataaaatatgtcatttaaatctcttttatctaatttaaggaacttaagattttcttcccaaaatttcacaaggtaggaaaatgatgtactttatttttgtgaatgatattgcactgtagatcttgctgtttcttgctttgttctcaattaaatatcatgtttcctctacagctttatagacattttgatcagattaaggacattctaattatagttctttaaggtgtttttaaaatcatatgtaggtattgaattttattaaatgctttttcctgcatgtattgggatactcacatgatctgttcttaaaattataattgatttctaattttaaaccatccttgcattcgtggaataaaactcagcctgaaggtgtggctggagagatggcttgttgctcttgcagaggacccaagttcagatcctattctctgtatatgcaaatacctgtattctcacaccccaacatacacacacacacacacacacacacacacacacactaccactcttacccacttgttttttacatttctatcttagtattttatgtgtataagtgttttgcctggtgctcactgtggtcagaagagggcacaggattccctgagactagaattacaaatggtttagaatggccatatgggaaatcctctagatttcccccactgtagtaagatattcacttaggtgatcctgtcccaaagtcagccatcatccttattttttttctttctttccatctgatatccaatacttttggctcaatttttaacataaatcttaactatcacaacttatcagatttcaactgctactgtcctggttaaagccttcatcatctatctttcttcaactgctgccaggacctctggaccagccagttcttcattcttcactggcaacataggttttatggtgacagctagtgactcaaatatttatcaagggcttctcatctcaaaataatctcctagttcttttggtggcctaggtctctctccagtcacactggcctccttagtaaggcaggcatagtccttccttagagtgtttaaacttgcctagaatgttttccccaattacccatattgggagacgacatgagggcaaaagctagagggtatcataatagcacttcttttgtccttgccctatctatttcaaagtctttatctctgtgcaaaattttaagttctactttcttgtatgtttagtatgactcttccttaccaggagtctagtttgtctccttgttcagtactaaaacagtgcctagcaaataaatgaatagagaggggagccaaatttgaatcagaaagtctcttgttgcatagtgtttaaaaaacaaacaaagaaagaaagtctcttgttgagcatttgtttagcacaaagagcattggatgctgactggtatcagggtaaggctgctttgacaatgctccctctggcctcactcccttttatacgtacttccatcaaaccatctgattcaacaatgacagaccgatctcttatgggcttggcacacaccatctgcccattataaacgtctgcaaagaccaaggtttgatatgttgattttactgtcagccttaagagtgcgacatctgctaatttagtgtaataatacaatcagtagaccctttaaaacaagtcccttggcttggaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcttaattaaagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgc

SEQ ID NO: 9 - DNA, 2108 bp, Artificial Sequence
  MeCP2_1 intron
ctctaccattacgttttatcctcagactctatctccccattttaaaggaatattatttttaaatgctacactctcattttttaaatggctccttttaattctactgctaaaatacttttggtacaatatgcctttttttctattttttttttttagtgcaagtataaaatatgtcatttaaatctcttttatctaatttaaggaacttaagattttcttcccaaaatttcacaaggtaggaaaatgatgtactttatttttgtgaatgatattgcactgtagatcttgctgtttcttgctttgttctcaattaaatatcatgtttcctctacagctttatagacattttgatcagattaaggacattctaattatagttctttaaggtgtttttaaaatcatatgtaggtattgaattttattaaatgctttttcctgcatgtattgggatactcacatgatctgttcttaaaattataattgatttctaattttaaaccatccttgcattcgtggaataaaactcagcctgaaggtgtggctggagagatggcttgttgctcttgcagaggacccaagttcagatcctattctctgtatatgcaaatacctgtattctcacaccccaacatacacacacacacacacacacacacacacacacactaccactcttacccacttgttttttacatttctatcttagtattttatgtgtataagtgttttgcctggtgctcactgtggtcagaagagggcacaggattccctgagactagaattacaaatggtttagaatggccatatgggaaatcctctagatttcccccactgtagtaagatattcacttaggtgatcctgtcccaaagtcagccatcatccttattttttttctttctttccatctgatatccaatacttttggctcaatttttaacataaatcttaactatcacaacttatcagatttcaactgctactgtcctggttaaagccttcatcatctatctttcttcaactgctgccaggacctctggaccagccagttcttcattcttcactggcaacataggttttatggtgacagctagtgactcaaatatttatcaagggcttctcatctcaaaataatctcctagttcttttggtggcctaggtctctctccagtcacactggcctccttagtaaggcaggcatagtccttccttagagtgtttaaacttgcctagaatgttttccccaattacccatattgggagacgacatgagggcaaaagctagagggtatcataatagcacttcttttgtccttgccctatctatttcaaagtctttatctctgtgcaaaattttaagttctactttcttgtatgtttagtatgactcttccttaccaggagtctagtttgtctccttgttcagtactaaaacagtgcctagcaaataaatgaatagagaggggagccaaatttgaatcagaaagtctcttgttgcatagtgtttaaaaaacaaacaaagaaagaaagtctcttgttgagcatttgtttagcacaaagagcattggatgctgactggtatcagggtaaggctgctttgacaatgctccctctggcctcactcccttttatacgtacttccatcaaaccatctgattcaacaatgacagaccgatctcttatgggcttggcacacaccatctgcccattataaacgtctgcaaagaccaaggtttgatatgttgattttactgtcagccttaagagtgcgacatctgctaatttagtgtaataatacaatcagtagaccctttaaaacaagtcccttggcttggaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcttaattaa

SEQ ID NO: 10 - DNA, 5802 bp, Artificial Sequence
  pPG35
ggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgaggccaggccggccgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 11 - DNA, 5629 bp, Artificial Sequence
  pPG36
ctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgaggccaggccggccgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 12 - DNA, 1782 bp, Homo sapiens
atgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtga

SEQ ID NO: 13 - protein, 593 aa, Homo sapiens
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 14 - DNA, 6 bp, Artificial Sequence
  Age1 restriction site
accggt

SEQ ID NO: 15 - DNA, 588 bp, Woodchuck hepatitis virus
tcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgca

SEQ ID NO: 16 - DNA, 198 bp, Artificial Sequence
  PolyA signal sequence
gatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttag

SEQ ID NO: 17 - DNA, 4566 bp, Artificial Sequence
  AAVTT-pPG36
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacctctgctagcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcaccggtgcgctccctcctctcggagagagggctgtggtaaaacccgtccggaaattggccgccgctgccgccaccgccgccgccgccgccgcgccgagcggaggaggaggaggaggcgaggaggagagactgtgagtgggaccgccaaggccgcgggcggggacccttgctggggggcgggtaggggcgggacgtggcgcgggaggggcccgcggggtcgggcgacacggctggcggttggcgtccctcctctctaccctccccctccctctgccgccggtggtggctttctccactcgtctcccgcaatcgcgagcgacggttctcagcgcgatctccctggagccaccttcgattgacgccctcccgctgcccgccccatctgtgcgcatcctaggccccagctgtgcaagcgcccttgtcgtctgggcttcgccagttggggctgcgcgcgctcctgcccttcttggggctttgggcctcggcactgtcgcgcgcccgcggtcccggcctctccctggatcgcgctgtccccttctccctcgcgcgcccccactcccgttacttgctcccccctcacacacacagactggcgcgcgtgcgcagtccatctcccgttgggagagtgcgccacaagggctcctgagctcttacccccatctctgggttttgctccctcctcctcctctcccattccgtgactttttgcccccactgcaagcgagtcggtccatcagctccattccccacttggcaggaacaagttgagggttattgtccacccacaaaaaggactagacattttgttcctaggtcccacaactcatcataaagagttggttgtagttctcatcaggaaccgtgggcaagggactgtgcgttcctcagcactcgaagctcttccgtgagaccttgcccgcagggtgctctggttctttggggttgctgtgctgtggcttcggaatttgagcgtcttcccaccctccctcccctcccttcgccagcgttctgtctacaagaaagaataggcaggtgtccttggatatcgtagttgctaatcgcctatacactgttctattacacctttctgctaaggatagggtttttggttttggttttggttttgttccccaccctccagtttggtttagttttggttttggcatttagggttttttgggggggagtaatatcttgtggtaaagacccatctgacccaagataccttttttctcatactggaaccctaggcagcagttgctatttccctgagttagcaatagttttacagtattttgaggccttttgtccataattctcacggaatccctcagggatcagattagctgctgttgggatcaggaaattgggttacaccgctgaaatctcttgctggggcccttgttttgaattggaaagtcaggaggctggaacgaaggctcacaagttaacagtgccagctgctcttccagaagccctggattcagtcccaccaatccatcgcgggtcacaaccatctgtaacttcagtcccaaggggtccgaagccctcttctggctttgccctattattttatttatcttatctgtttttgtcttgtcatctggcaagcccagggggccattgggtgcaacttataaactgacttctgtatcttaagaagccaaccatacagtgcttacattccagaaaaaaaatctgccactttaacagcactagaactagggtttagagaagtatcataaaggtcaaatatctttgaccaatatcaccagcaacctaaagctgttaagaaatctttgggccccagcttgacccaaggatacagtatcctagggaagttaccaaaatcagagatagtatgcagcagccaggggtctcatgtgtggcactcaagctcacctatactcactactgtgcagacagctgtgttctctgtaatacttacatatttgtttaatacttcagggaggaaaagtcagaagaccaggatctccagggcctcaaccggtggcccaggcggccaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgaggccaggccggccgaattcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagggatcctcaggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 18 - DNA, 6486 bp, Artificial Sequence
  AAVTT-p1PG36
aataaattgcagtttcatttgatgctcgatgagtttttctaactcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcaaggctgactgcagggcgagaagattgcgagctgtgcggctgagttgacgtatctgtgctggatgattactcataacggcaccgctatcaaacgtgccacgttcatgtcctacagcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacctctgctagcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcaccggtgcgctccctcctctcggagagagggctgtggtaaaacccgtccggaaattggccgccgctgccgccaccgccgccgccgccgccgcgccgagcggaggaggaggaggaggcgaggaggagagactgtgagtgggaccgccaaggccgcgggcggggacccttgctggggggcgggtaggggcgggacgtggcgcgggaggggcccgcggggtcgggcgacacggctggcggttggcgtccctcctctctaccctccccctccctctgccgccggtggtggctttctccactcgtctcccgcaatcgcgagcgacggttctcagcgcgatctccctggagccaccttcgattgacgccctcccgctgcccgccccatctgtgcgcatcctaggccccagctgtgcaagcgcccttgtcgtctgggcttcgccagttggggctgcgcgcgctcctgcccttcttggggctttgggcctcggcactgtcgcgcgcccgcggtcccggcctctccctggatcgcgctgtccccttctccctcgcgcgcccccactcccgttacttgctcccccctcacacacacagactggcgcgcgtgcgcagtccatctcccgttgggagagtgcgccacaagggctcctgagctcttacccccatctctgggttttgctccctcctcctcctctcccattccgtgactttttgcccccactgcaagcgagtcggtccatcagctccattccccacttggcaggaacaagttgagggttattgtccacccacaaaaaggactagacattttgttcctaggtcccacaactcatcataaagagttggttgtagttctcatcaggaaccgtgggcaagggactgtgcgttcctcagcactcgaagctcttccgtgagaccttgcccgcagggtgctctggttctttggggttgctgtgctgtggcttcggaatttgagcgtcttcccaccctccctcccctcccttcgccagcgttctgtctacaagaaagaataggcaggtgtccttggatatcgtagttgctaatcgcctatacactgttctattacacctttctgctaaggatagggtttttggttttggttttggttttgttccccaccctccagtttggtttagttttggttttggcatttagggttttttgggggggagtaatatcttgtggtaaagacccatctgacccaagataccttttttctcatactggaaccctaggcagcagttgctatttccctgagttagcaatagttttacagtattttgaggccttttgtccataattctcacggaatccctcagggatcagattagctgctgttgggatcaggaaattgggttacaccgctgaaatctcttgctggggcccttgttttgaattggaaagtcaggaggctggaacgaaggctcacaagttaacagtgccagctgctcttccagaagccctggattcagtcccaccaatccatcgcgggtcacaaccatctgtaacttcagtcccaaggggtccgaagccctcttctggctttgccctattattttatttatcttatctgtttttgtcttgtcatctggcaagcccagggggccattgggtgcaacttataaactgacttctgtatcttaagaagccaaccatacagtgcttacattccagaaaaaaaatctgccactttaacagcactagaactagggtttagagaagtatcataaaggtcaaatatctttgaccaatatcaccagcaacctaaagctgttaagaaatctttgggccccagcttgacccaaggatacagtatcctagggaagttaccaaaatcagagatagtatgcagcagccaggggtctcatgtgtggcactcaagctcacctatactcactactgtgcagacagctgtgttctctgtaatacttacatatttgtttaatacttcagggaggaaaagtcagaagaccaggatctccagggcctcaaccggtggcccaggcggccaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgaggccaggccggccgaattcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagggatcctcaggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcactgtcattagcaactccttgtccttcgatctcgtcaacaacagcttgcagttcaaatacaagacccagaaggcgactattctggaagcgagcttgaagagttaacctgcagagagcccccgcagtgtcgacaattaatcatcggcatagtatatcggcatagtataatacgacaaggtgaggaagtaaaaaatgagccatatccaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatg

SEQ ID NO: 19 - DNA, 10353 bp, Artificial Sequence
  AAVTT-p2PG36
gaagcattttgttaaaattcgcgttaaatttttgttaaatcagctattttttaaccaataggccgaaatcggcaaaatcccttgtaaatcaaaagaatagaccgagatagggttgagtgttgttccagtttggaacaagagtccactattaaagaacgtggactccaacgtcaaagggcgaaaaaccgtctatcagggcgttggcccactacgtgaaccttcaccctaatcaagttttttggggtcgaggtgccgtaaagcactaaatcggaaccctaaagggagcccccgatttagagcttgacggggaaaccggcgaacgtggcgagaaaggaagggaagaaagcgaaaggagcgggcgctagggcgctggcaagtgtagcggtcacgctgcgcgtaaccaccacacccgccgcgctaagcgccgctacagggcgcgtcccttcgccttcaggctgcgtcgagtactgtactgtgagccagagttgcccggcgctctccggctgcggtagttcaggcagttcaatcaactgtttaccttgtggagcgactccagaggcacttcaccgcttgccagcggcttacgatccagcgccacgatccagtgcaggagatcgttatcgctatacggaacaggtattcgctggtcacttcgataaggtttgcccggataaacggaactggaaaaactgctgctggtgttttgcttccgtcagtgctggatcggcgtgcggtcggcaaagaccagaccgttctaacagaactggcgattgttcggcgtatcgccaaaatcaccgccgtaagccgaccacgggttgccgttttcagcaggatttaatcagcgactgatccacccagtcccagacgaagccgccctgtaaacggggatactgacgaaacgcctgccagtatttagcgaaaccgccaagactgttacccaagcgtgggcgtattcgcaaaggatcagcgggcgcgtctctccaggtagcgaaagccttttttgatcgacctttcggcacagccgggaagggctggtcttcaaccacgcgcgcgtacaacgggcaaataatatcggtggccgtggtgtcggctccgccgccttcaactgcaccgggcgggaaggatcgacagatttgatccagcgatacagcgcgtcgtgattagcgccgtggcctgattcaattccccagcgaccagtagatcacactcgggtgattacgattgcgctgcaccagtcgcgttacggttcgctcttcgccggtagccagcgcggatcacggtcagacgattcgttggcacgatccgtgggtttcaatactggcttcaaaccaccactaacaggccgtagcggtcgcacagcgtgtaccacagcggttggttcggataatcgaacagcgcacggcgttaaagttgttctgcttcaacagcaggatattctgcaccttcgtctgctcttcctaacctgaccaagcagaggatctgctcgtgacggttaatcctcgaatcagcaacggcttgccgttcagcagcagcagaccaagttcaatccgcacctcgcggaaaccgacaacgcaggcttctgcttcaatcagcgtgccgtcggcggtgtgcagttcaaccaccgcacgatagagattcgggatttcggcgctccacagtttcgggttttcgacgttcagacgtagtgtgacgcgatctgcaaaccaccacgctcaacgataatttcaccgccgaaaggcgcggtgccgctggcgacctgcgtttcaccctgccagaaagaaactgttacccgtaggtagtcacgcaactcgccgcacactgaacttcagcctccagtacagcgcggctgaaatcgtcttaaagcgagtggcaactggaaatcgctgatttgtgtagtcggtttagcagcaacgagacttcacggaaaatccgctaatccgccacagatcctgatcttccagataactgccgtcactccaacgcagcaccttcaccgcgaggcggttttctccggcgcgtaaaaatcgctcaggtcaaattcagacggcaaacgactgtcctggccgtaaccgacccagcgcccgttgcaccacagattgaaacgccgagtttacgcctcaaaaataattcgcgtctggccttcctgtagccagctttcacaactataatagtgagcgagtaacaacccgtcggattctccgtgggaacaaacggcggattgaccgtatagggataggttacgttggtgtagtagggcgctccgtaaccgtgctactgccagtttgaggggacgacgacagtatcggcctcaggaagatcgcactccagccagctttccggcaccgcttctggtactggaaaccaggcaaagcgcctatcgcctatcaggctgcacaactgttgggaagggcgatctgtgcgggcctcttcgctattacgccagcttgcgaaagggggtagtgctgcaaggcgattaagttgggtaacgccagggttttcccagtcacgacgttgtaaaacgacgggatctatcagcgctacatgttctttcctgcgttatcccctgattctgtggataaccgtattaccgcctttgagtgagctgataccgctcaaggctgactgcagggcgagaagattgcgagctgtgcggctgagttgacgtatctgtgctggatgattactcataacggcaccgctatcaaacgtgccacgttcatgtcctacagcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacctctgctagcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcaccggtgcgctccctcctctcggagagagggctgtggtaaaacccgtccggaaattggccgccgctgccgccaccgccgccgccgccgccgcgccgagcggaggaggaggaggaggcgaggaggagagactgtgagtgggaccgccaaggccgcgggcggggacccttgctggggggcgggtaggggcgggacgtggcgcgggaggggcccgcggggtcgggcgacacggctggcggttggcgtccctcctctctaccctccccctccctctgccgccggtggtggctttctccactcgtctcccgcaatcgcgagcgacggttctcagcgcgatctccctggagccaccttcgattgacgccctcccgctgcccgccccatctgtgcgcatcctaggccccagctgtgcaagcgcccttgtcgtctgggcttcgccagttggggctgcgcgcgctcctgcccttcttggggctttgggcctcggcactgtcgcgcgcccgcggtcccggcctctccctggatcgcgctgtccccttctccctcgcgcgcccccactcccgttacttgctcccccctcacacacacagactggcgcgcgtgcgcagtccatctcccgttgggagagtgcgccacaagggctcctgagctcttacccccatctctgggttttgctccctcctcctcctctcccattccgtgactttttgcccccactgcaagcgagtcggtccatcagctccattccccacttggcaggaacaagttgagggttattgtccacccacaaaaaggactagacattttgttcctaggtcccacaactcatcataaagagttggttgtagttctcatcaggaaccgtgggcaagggactgtgcgttcctcagcactcgaagctcttccgtgagaccttgcccgcagggtgctctggttctttggggttgctgtgctgtggcttcggaatttgagcgtcttcccaccctccctcccctcccttcgccagcgttctgtctacaagaaagaataggcaggtgtccttggatatcgtagttgctaatcgcctatacactgttctattacacctttctgctaaggatagggtttttggttttggttttggttttgttccccaccctccagtttggtttagttttggttttggcatttagggttttttgggggggagtaatatcttgtggtaaagacccatctgacccaagataccttttttctcatactggaaccctaggcagcagttgctatttccctgagttagcaatagttttacagtattttgaggccttttgtccataattctcacggaatccctcagggatcagattagctgctgttgggatcaggaaattgggttacaccgctgaaatctcttgctggggcccttgttttgaattggaaagtcaggaggctggaacgaaggctcacaagttaacagtgccagctgctcttccagaagccctggattcagtcccaccaatccatcgcgggtcacaaccatctgtaacttcagtcccaaggggtccgaagccctcttctggctttgccctattattttatttatcttatctgtttttgtcttgtcatctggcaagcccagggggccattgggtgcaacttataaactgacttctgtatcttaagaagccaaccatacagtgcttacattccagaaaaaaaatctgccactttaacagcactagaactagggtttagagaagtatcataaaggtcaaatatctttgaccaatatcaccagcaacctaaagctgttaagaaatctttgggccccagcttgacccaaggatacagtatcctagggaagttaccaaaatcagagatagtatgcagcagccaggggtctcatgtgtggcactcaagctcacctatactcactactgtgcagacagctgtgttctctgtaatacttacatatttgtttaatacttcagggaggaaaagtcagaagaccaggatctccagggcctcaaccggtggcccaggcggccaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgaggccaggccggccgaattcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagggatcctcaggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcactgtcattagcaactccttgtccttcgatctcgtcaacaacagcttgcagttcaaatacaagacccagaaggcgactattctggaagcgagcttgaagagttaacctgcagagagcccccgcagtgtcgactgttaaccttaattaaccatttaaatcgtagtgcaaccgaacgcgaccgttggtcagaagccgggcaaatcagcgcctggcagcagtggcgtctggcggaaaacctcagtgtgacgctccccgccgcgtcccacgcttgttcccggatctgaccaccagcgaaatccgatttttgcaccgagctgggtaataagcgttggcaatttaaccgccagtcaggctttctttcacagtgtggattggcgataaaaaacaactgctgacgccgctgcgcgatcagttcacccgttcaccgctggataacgacttggcgtaagtgaagcgacccgtaagaccctaacgcctgggtcgaacgctggaaggcggcgggccaaaccaggccgaagcagcgttgttgcagttcacggcagatacacttgctgttgcggtgctgattacgaccgctcactcgtggcagcaacaggggaaaaccttatttatcagccggaaaacctaccggattgttggtagtggtcaataggcgattaccgttgtgttgaagtggcgagcgatacaccgcttccggcgcggattggcctgaactgccaactggcgcaggtagcagagcgggtaaactggctcggattagggccgcaagaaaactatcccgaccgccttactgccgcctgttttgaccgctgggatctgccaagtcagacagtatagcccgtacgtcttcccgagcgaaaacggtctgcgctgcgggacgcgcgaattgaatttggcccacaccagtggcgcggcgacttccagttcaatatcagccgctacagtgaacagcaactgttggaaaccagccttcgccaactgctgcacgcggaagaaggcactggctgaatatcgacggtttccagttggggattggtggcgacgactcctggagcccgtcagtatcggcggacttccaactgagcgccggtcgctaccttaccagttggtctggtgtcaaaaagcgtccgcttgagtctagcgatcgcgcgcagatctgtcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaatttaaagcccaatacgcaaaccgcctctccccgcgcgttggcc

SEQ ID NO: 20 - DNA, 128 bp, Artificial Sequence
  5′ ITR
gcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 21 - DNA, 18 bp, Artificial Sequence
  5′ adjacent fragment
tgtagttaatgattaacc

SEQ ID NO: 22 - DNA, 17 bp, Artificial Sequence
  3′ adjacent fragment
gttaatcattaactaca

SEQ ID NO: 23 - DNA, 128 bp, Artificial Sequence
  3′ ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 24 - DNA, 6 bp, Artificial Sequence
  Kozak sequence
gccacc